(19) United States
(12) Patent Application Publication
(10) Pub. No.: US 2025/0347685 A1
(43) Pub. Date: Nov. 13, 2025

(54) DONOR-ACCEPTOR CONJUGATED OLIGOELECTROLYTES FOR CELL LABELLING AND METHODS THEREOF

(71) Applicant: National University of Singapore, Singapore (SG)

(72) Inventors: Guillermo Carlos BAZAN, Singapore (SG); Cheng ZHOU, Singapore (SG); Sarah COX-VAZQUEZ, Singapore (SG); Wan Ni Geraldine CHIA, Singapore (SG)

(73) Assignee: National University of Singapore, Singapore (SG)

(21) Appl. No.: 18/553,840

(22) PCT Filed: Aug. 16, 2022
(86) PCT No.: PCT/SG2022/050582

§ 371 Date: Oct. 3, 2023

(30) Foreign Application Priority Data
Aug. 16, 2021 (SG) 10202108936X

(51) Int. Cl. G01N 33/533; C07C 217/20; C07D 409/10; C07D 409/14; C07D 413/14; C07D 417/14; C07D 421/14; C07D 513/04; C07D 519/00; C09K 11/06

(52) U.S. Cl. CPC G01N 33/533; C07C 217/20; C07D 409/10; C07D 409/14; C07D 413/14; C07D 417/14; C07D 421/14; C07D 513/04; C07D 519/00; C09K 11/06; C09K 2211/1007; C09K 2211/1037; C09K 2211/1051; C09K 2211/1055

(57) ABSTRACT

The present disclosure relates to compounds of Formula (I) and their methods of use thereof. The compounds of Formula (I) are conjugated oligoelectrolytes and are suitable for use as a membrane probe to label and/or detect cells and/or lipid vesicles and thus in flow cytometry applications. The present disclosure also relates to a flow system for detecting and/or quantifying cells and/or lipid vesicles.

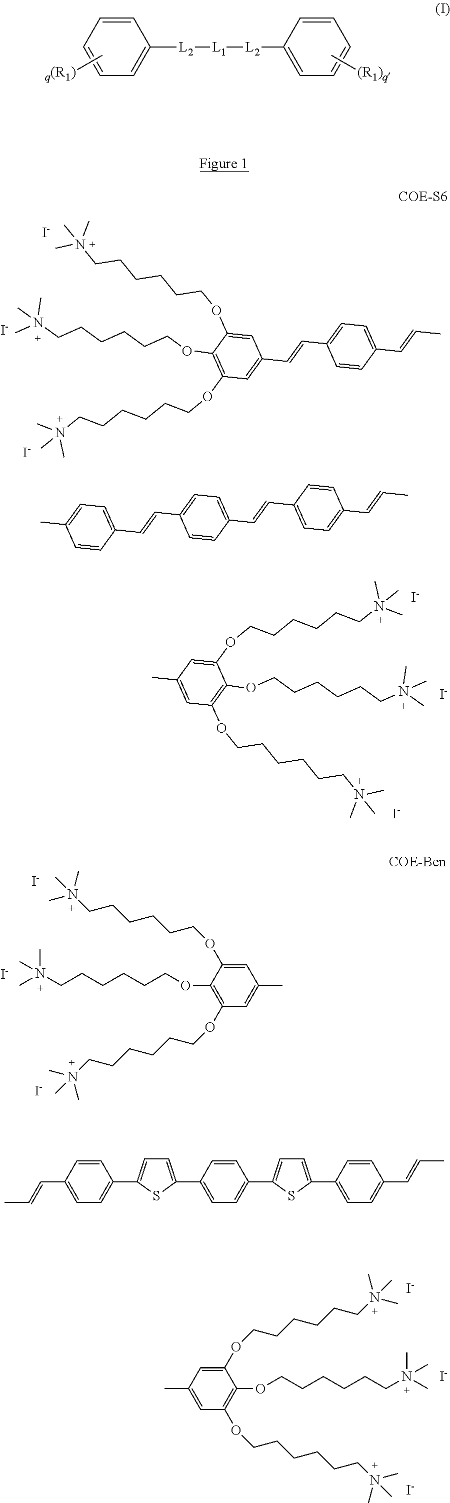

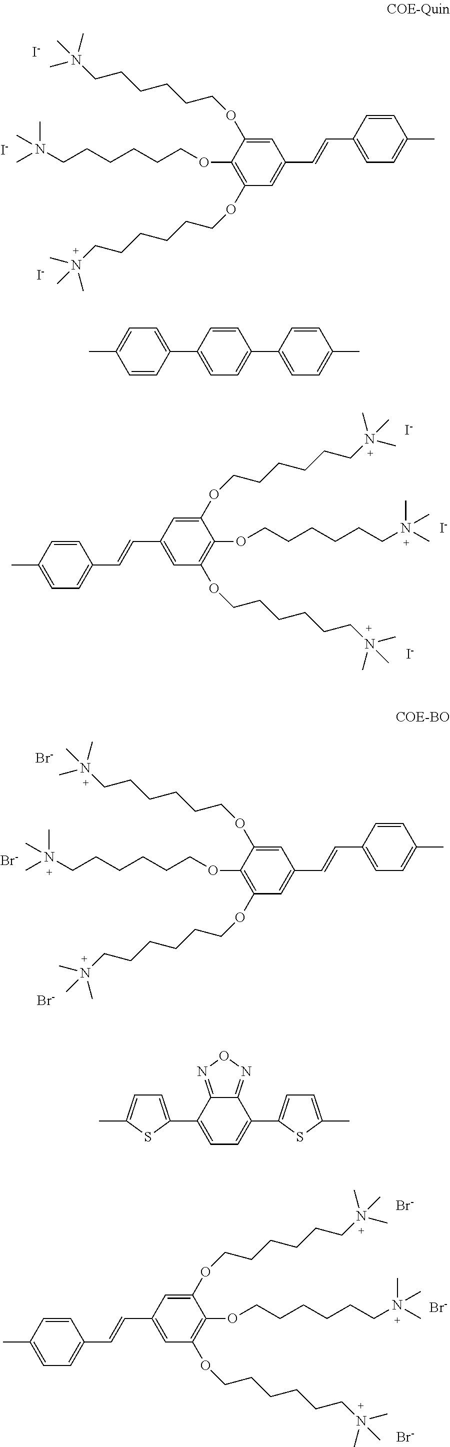

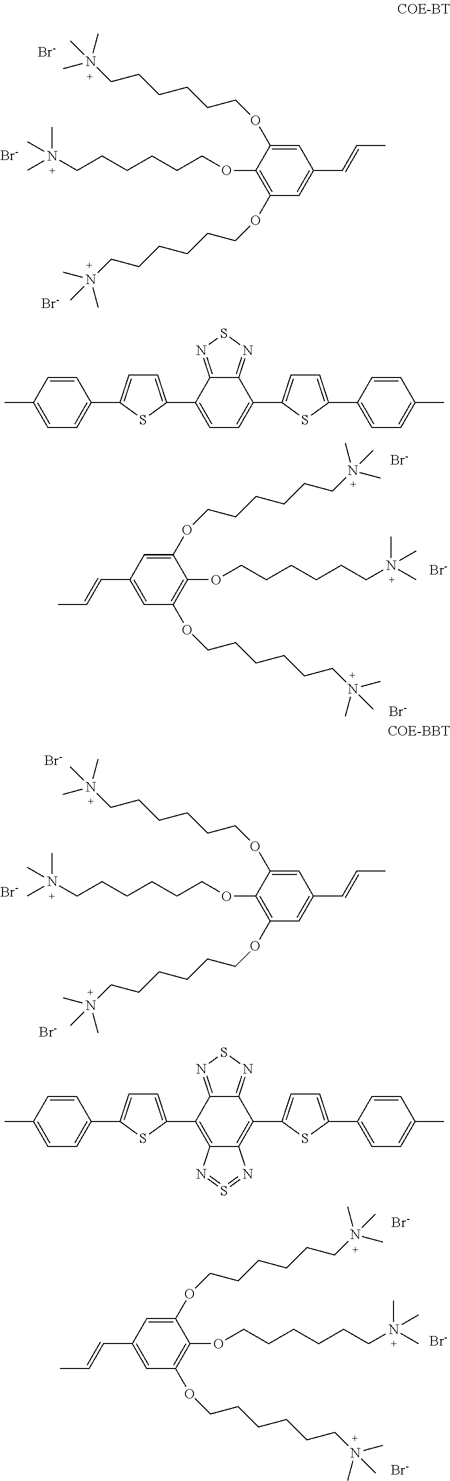

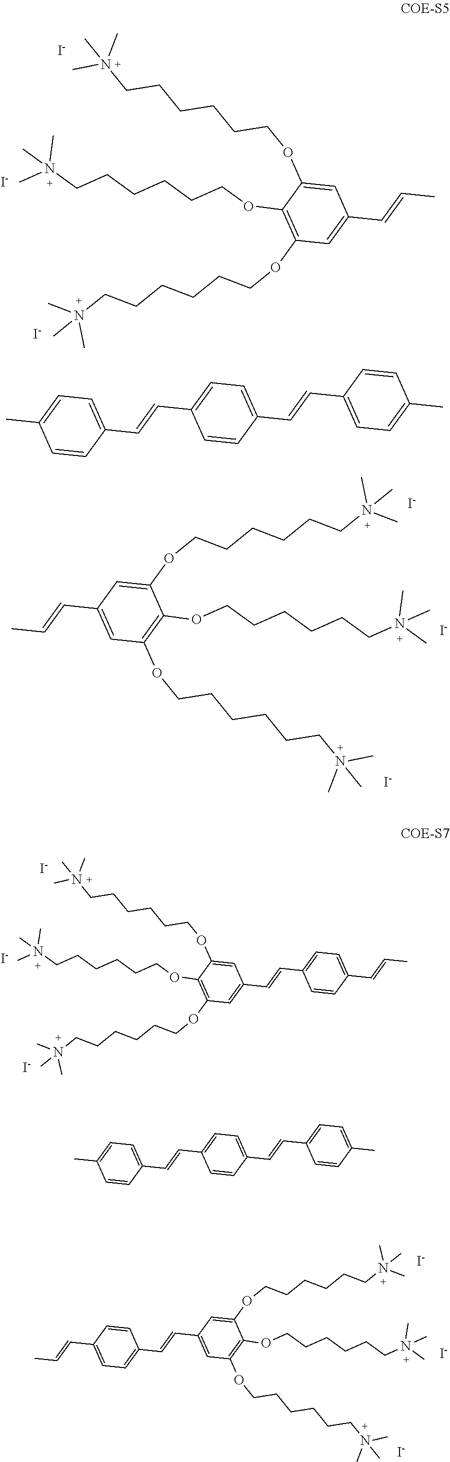

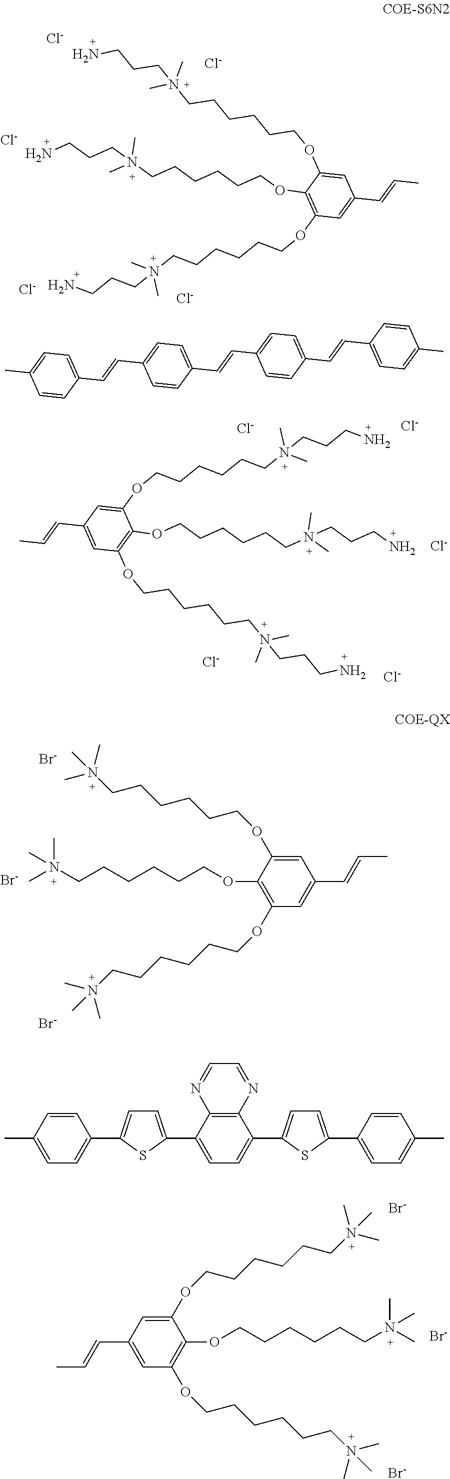

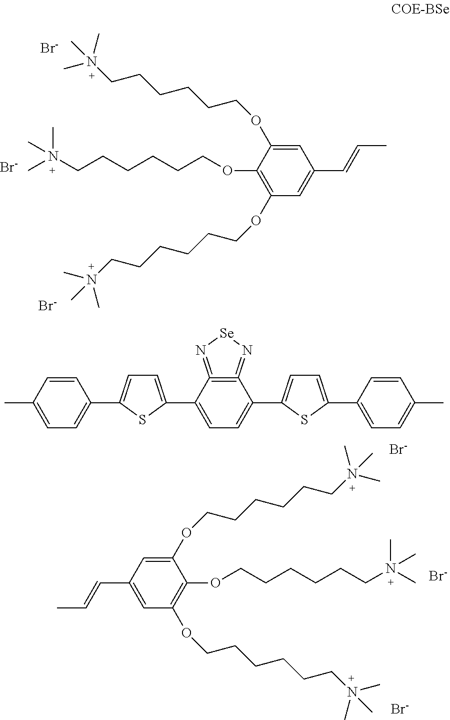

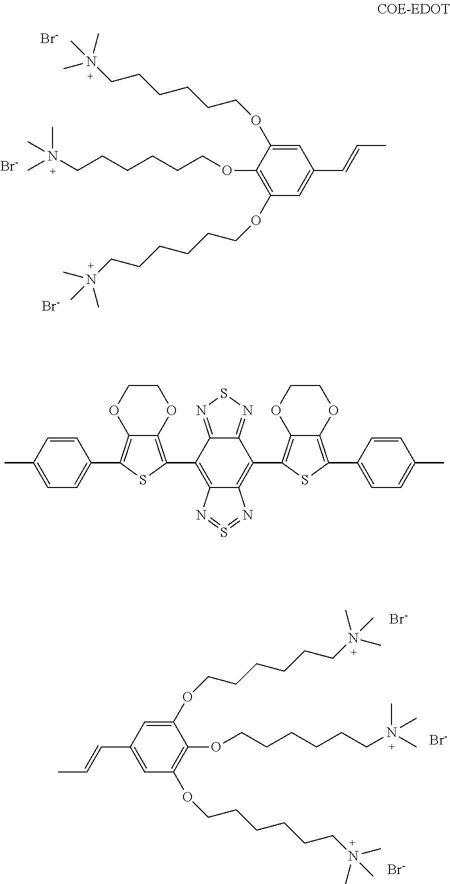

This application is a national phase application under 35 U.S.C. § 371 of International Application No. PCT/SG2022/050582, filed Aug. 16, 2022, the entire contents of which are hereby incorporated by reference. International Application No. PCT/SG2022/050582 claims benefit of Singaporean Application No. 10202108936X, filed Aug. 16, 2021.

### FIELD

The present disclosure relates to conjugated oligoelectrolytes and their methods of use thereof. In particular, the conjugated oligoelectrolytes are suitable for use as a membrane probe and thus in flow cytometry applications.

### BACKGROUND

Flow cytometry is a high-throughput laboratory technique to rapidly count, recognize, and sort individual cells, microbes and particles. It is used in clinical and research characterisation in many disciplines, including cancer biology, immunology, microbiology, and virology. Flow cytometry utilizes a microfluidic system, in which individual cells or particles flow into a stream and are quickly passed through a laser light source, that is then being analyzed via fluorescence or light scattering. Fluorescent proteins, fluorescently labelled antibodies, or structure-specific dyes such as DNA, or lipid membrane specific dyes can be used for measuring the unique properties of individual cells or particles. Fluorescent labelling is important for a variety of reasons including understanding cell viability, identifying different cell types in heterogeneous mixtures, measuring expression of antigens or proteins, cell cycle analysis, and understanding membrane integrity, to name a few.

As flow cytometry is heavily dependent on the fluorescence tag, there can be many issues faced by the technician depending on the fluorescent label used, or if multiple fluorescent labels are required. For example, the fluorescence intensity can be weak or if the fluorescence label is degraded due to exposure to light. Further, there are few fluorophore binding sites, or effective number of dyes, on the recognition probe. The coupling of the fluorescence label with the desired cell component may be weak. The fluorescent signal may be overly saturated when the fluorescence label is not properly internalised by the cells. Under inappropriate incubation conditions, the fluorescence label may aggregate and thus self-quench. High background or non-specific staining can also impede the detection method. When more than one fluorescence labels are used, the emitted signals may overlap causing the results to be confusing or even uninterpretable. Some fluorescence labels are also toxic to cells such that only a short working window is provided to perform flow cytometry.

There is therefore a need for molecules which can act as fluorescence labels or dyes for use in flow cytometry. There is a further need for fluorescence molecules which can preferentially target microbes, especially bacterial cells.

Accordingly, it would be desirable to overcome or ameliorate at least one of the above-described problems.

### SUMMARY

The present invention is predicated on the discovery that certain conjugated oligoelectrolytes (COEs) have differential membrane binding and are therefore advantageous for use as fluorescence membrane probes. In particular, the inventors have found that when the conjugated moieties along the backbone of COE is modified, the emitted fluorescence signal can be tuned to a certain wavelength. Further, the Stokes shift (difference between the peak excitation and peak emission) can be tuned. By further modifying the pendant chains at the terminal ends of the conjugated backbone, the selectivity to certain cell membranes can be tuned. The penetration of these COE compounds into the cell membrane allows the cells, liposomes, vesicles and other membrane-containing macrostructures to be analysed using flow cytometry.

The present invention provides a compound of Formula (I) or a salt or solvate thereof:

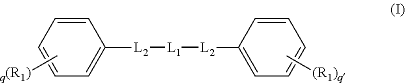

wherein
each R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
q is an integer selected from 1 to 5;
q′ is an integer selected from 1 to 5;
wherein
each L₂ is independently selected from optionally substituted ethylene, or optionally substituted phenylethylene;
L₁ is a π-conjugated core comprising monomeric unit A and monomeric unit D:

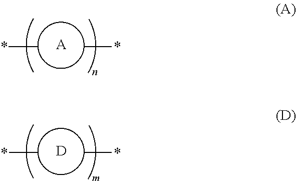

wherein
each A is independently selected from optionally substituted alkenylene, optionally substituted monocyclic heteroarylene or optionally substituted fused heteroarylene;
each D is independently selected from alkenylene, phenylene, optionally substituted fused arylene, optionally substituted monocyclic heteroarylene or optionally substituted fused heteroarylene;
n is an integer selected from 1 to 5;
m is an integer selected from 1 to 5;
wherein * represents a bond to another monomeric unit or to L₂;
wherein monomeric units A and monomeric units D are alternatively bonded to each other;
wherein the compound of Formula (I) has a substantially linear topology; and
wherein L₁ is not butadienylene, polyalkenylene, phenylalkenylene, polyphenylalkenylene.

The conjugated compound of Formula (I) comprises an alternating donor (D)/acceptor (A) composition of structural units (relative to each other) which allows for their emission, quantum yield, and stokes shift to be tunable across a much broader range than those that rely on the pi-conjugation of polyalkenylene or polyphenylalkenylene alone. Further, the molecular topology of the molecule allows it to accomplish fast self-assembly within the membranes of various cell and lipid types. A specific molecular fragment, for example phenylene, may be monomeric unit D or monomeric unit A, depending on the electron affinity or ionization potential of the adjacent groups.

In some embodiments, A is an electron accepting moiety.

In some embodiments, A is selected from

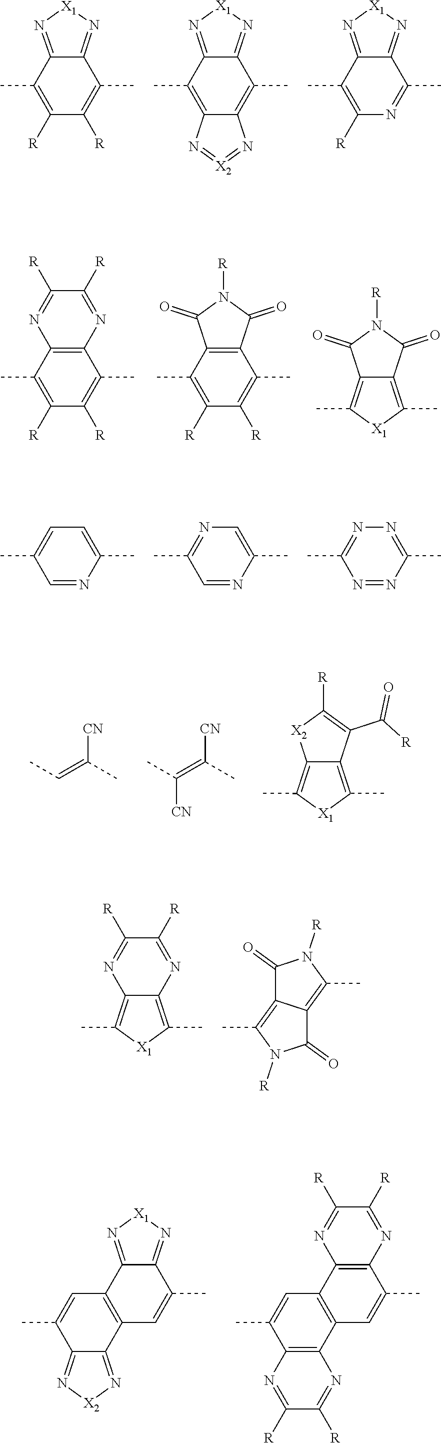

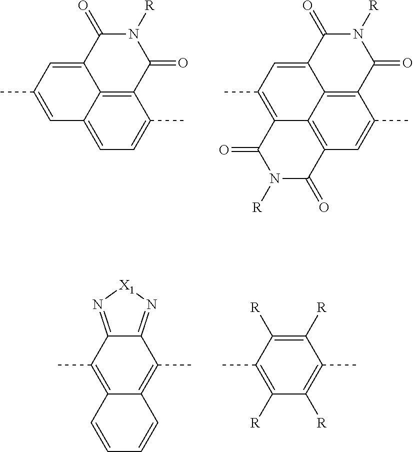

wherein [symbol] represents a bond to D or to L₂;
each X₁ is independently selected from C, O, N, S and Se;
each X₂ if present is independently selected from C, O, N, S and Se;
when X₂ is present, at least one of X₁ and X₂ is O, N or S;
each R is independently selected from H, halo, cyano, and optionally substituted alkyl.

In some embodiments, A is a moiety of Formula (II):

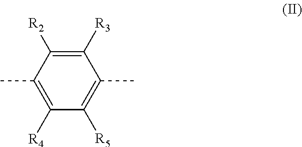

wherein [symbol] represents a bond to D or to L₂;
R₂, R₃, R₄ and R₅ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₂ and R₃ are linked to form optionally substituted heterocyclyl, optionally substituted heteroaryl; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl.

In some embodiments, A is

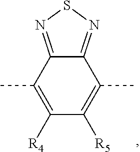

wherein R₄ and R₅ are as disclosed herein.

In some embodiments, A is

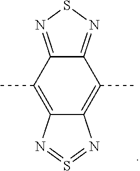

In some embodiments, D is an electron donating moiety.

In some embodiments, D is a moiety selected from

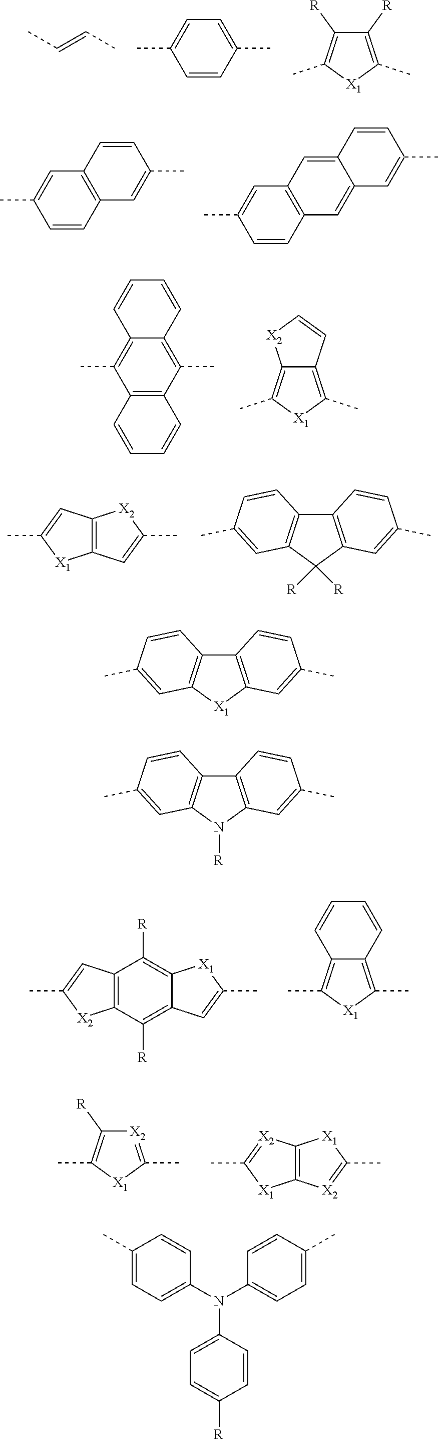

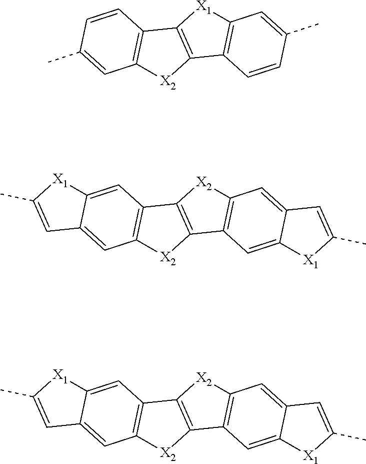

wherein [symbol] represents a bond to A or to L₂;
each X₁ is independently selected from C, O, N, S, and Se;
each X₂ if present is independently selected from C, O, N, S, and Se;
when X₂ is present, at least one of X₁ and X₂ is O, N or S;
R is independently selected from H, halo, cyano, and optionally substituted alkyl.

In some embodiments, D is an optionally substituted 5 membered heteroarylene.

In some embodiments, D is a moiety of Formula (III):

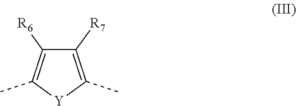

wherein Y is NR, O, or S;
R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl; and
R is selected from H, halo, cyano, and optionally substituted alkyl.

In some embodiments, D is

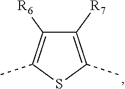

wherein R₆ and R₇ are as disclosed herein.

In some embodiments, D is

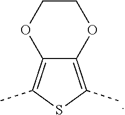

In some embodiments, L₁ is selected from:

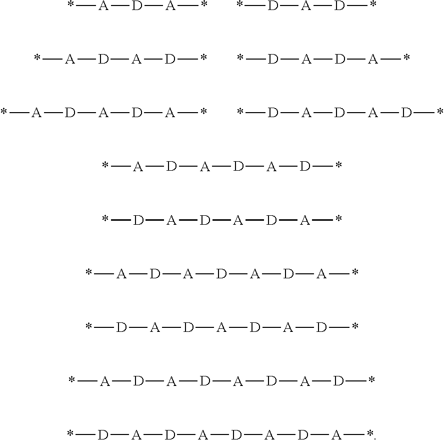

In some embodiments, each R₁ is independently selected from optionally substituted alkyl, optionally substituted alkoxy.

In some embodiments, each R₁ is independently selected from alkyl and alkoxy, each optionally substituted with amino, or alkylamino.

In some embodiments, each R₁ is independently C₃-C₈ alkoxy substituted with amino, or alkylamino.

In some embodiments, the optional substituent on L₁ is independently selected from halogen, cyano, optionally substituted alkyl, optionally substituted alkenyl or optionally substituted alkynyl.

In some embodiments, the compound of formula (I) is a compound of Formula (Ia):

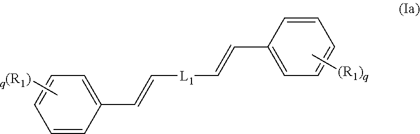

wherein L₁, A, D, R₁, n, m and q are as disclosed herein.

In some embodiments, the compound of formula (I) is a compound of Formula (Ib):

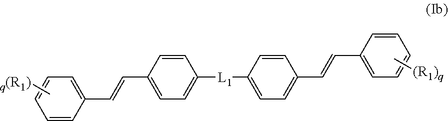

wherein L₁, A, D, R₁, n, m and q are as disclosed herein.

In some embodiments, the compound of formula (I) is a compound of Formula (Ib), wherein A is a moiety of Formula (II):

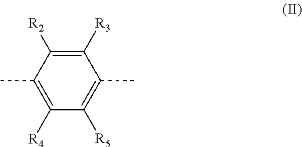

wherein [symbol] represents a bond to D or to L₂;
R₂, R₃, R₄ and R₅ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₂ and R₃ are linked to form optionally substituted heterocyclyl, optionally substituted heteroaryl; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl; and
D is a moiety of Formula (III):

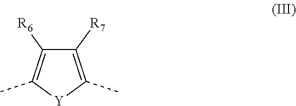

wherein Y is NR, O, or S;
R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl.

In some embodiments, the compound of formula (I) is a compound of Formula (Ib), wherein
A is

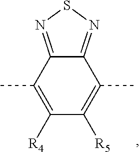

wherein R₄ and R₅ are as disclosed herein; and
D is

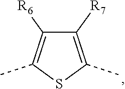

wherein R₆ and R₇ are as disclosed herein.

In some embodiments, the compound of Formula (I) is a compound of Formula (Ic):

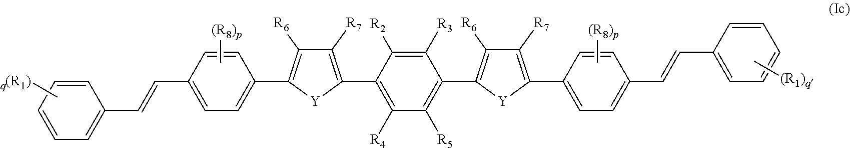

each R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
R₂, R₃, R₄ and R₅ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₂ and R₃ are linked to form optionally substituted heterocyclyl, optionally substituted heteroaryl; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl;
R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl;
each R₈ is independently selected from halogen, cyano, optionally substituted alkyl, optionally substituted alkenyl or optionally substituted alkynyl;
Y is NR, O, or S;
each p is an integer independently selected from 0 to 4;
q is an integer selected from 1 to 5; and
q′ is an integer selected from 1 to 5.

In some embodiments, the compound of Formula (I) is a compound of Formula (Id):

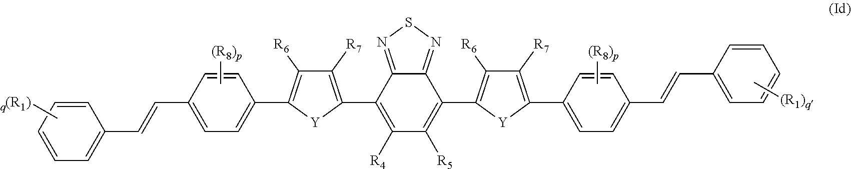

wherein R₁, R₄, R₅, R₆, R₇, R₈, Y, p, q and q′ are as disclosed herein.

In some embodiments, the compound of Formula (I) is a compound of Formula (Ie):

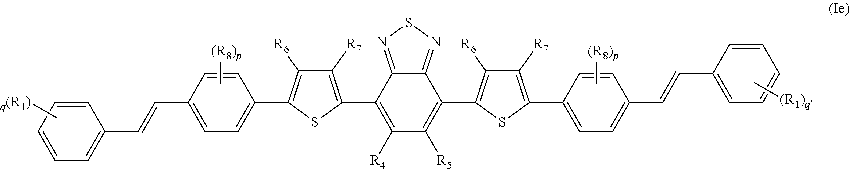

wherein R₁, R₄, R₅, R₆, R₇, R₈, p, q and q′ are as disclosed herein.

In some embodiments, the compound of Formula (I) is a compound of Formula (If):

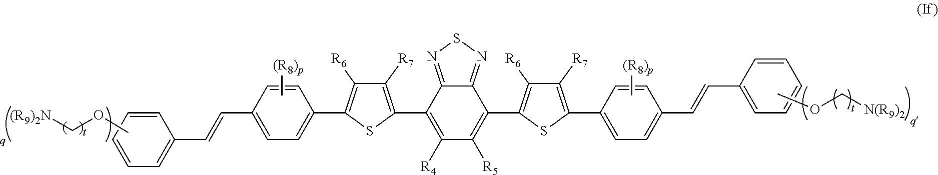

wherein R₄, R₅, R₆, R₇, R₈, R₉, p, q and q′ are as disclosed herein;
each R₉ is independently H or optionally substituted alkyl; and
each t is an integer independently selected from 1 to 8.

In some embodiments, the compound of Formula (I) is selected from

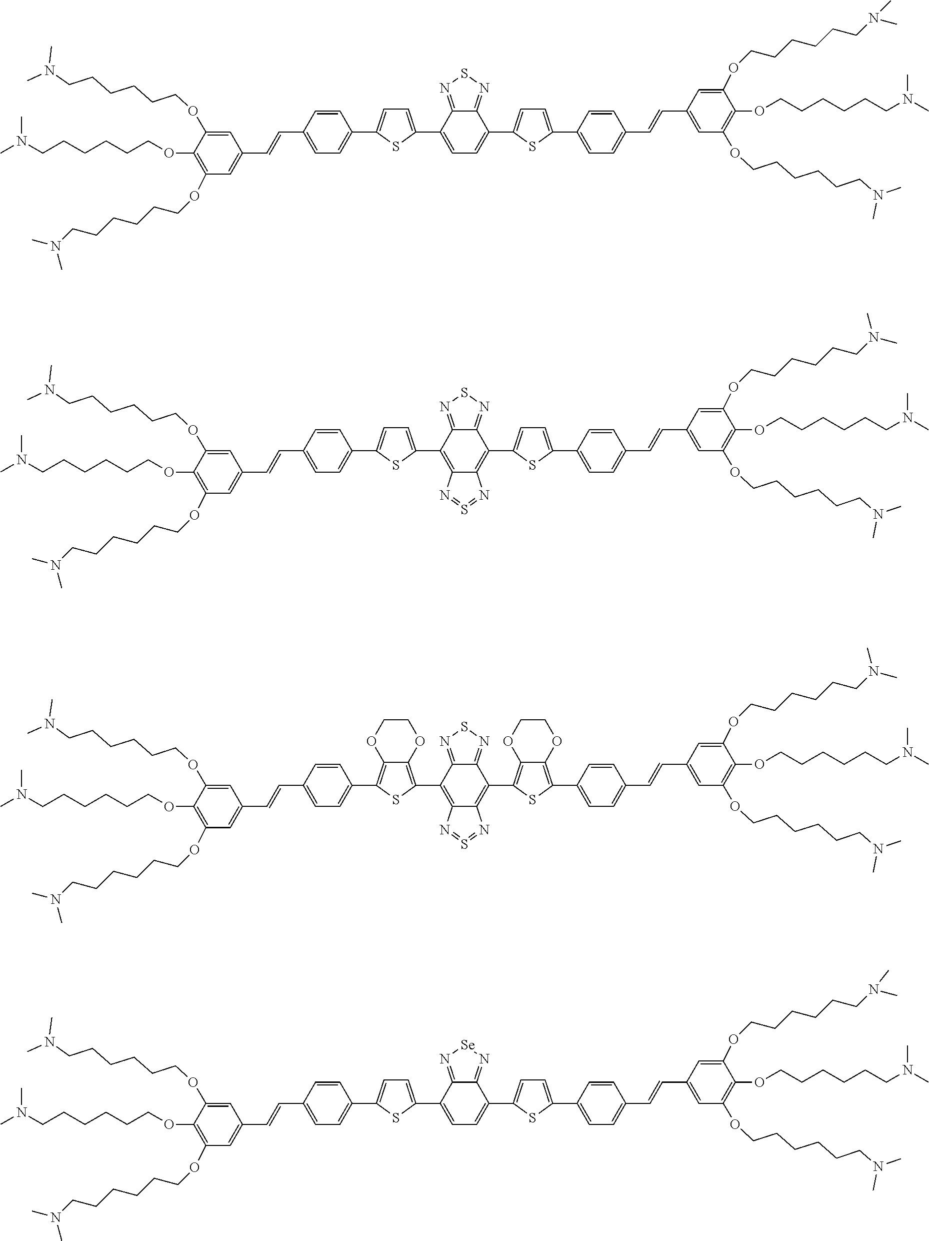

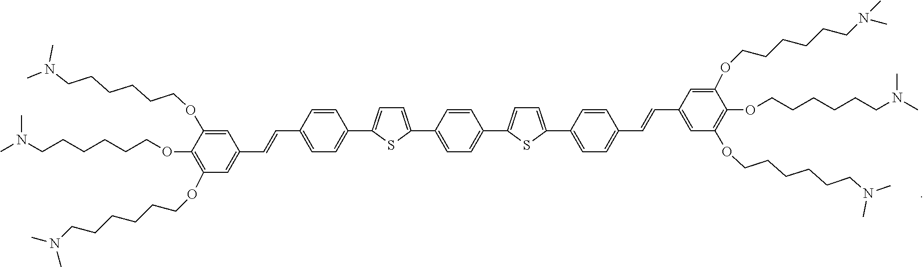

The present invention also provides a compound of Formula (Ig) or a salt or solvate thereof:

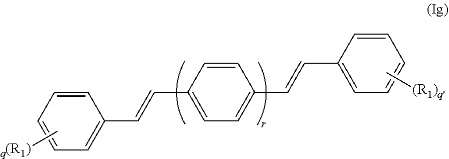

wherein
each R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
q is an integer selected from 1 to 5;
q; is an integer selected from 1 to 5; and
r is an integer selected from 1 to 5.

The present invention also provides a method of labelling a cell and/or a lipid vesicle, comprising:
a) incubating a compound of Formula (I), sub-Formula (Ia-Ig) or a salt or solvate thereof with the cell and/or a lipid vesicle.

The present invention also provides a method of detecting a cell and/or a lipid vesicle using a fluorescence detector, comprising:
a) incubating a compound of Formula (I), sub-Formula (Ia-Ig) or a salt or solvate thereof with the cell and/or lipid vesicle; and
b) passing the cell through the fluorescence detector.

The present invention also provides a method of detecting a cell and/or a lipid vesicle using a flow cytometer, comprising:
a) incubating a compound of Formula (IV) or a salt or solvate thereof with the cell and/or a lipid vesicle;

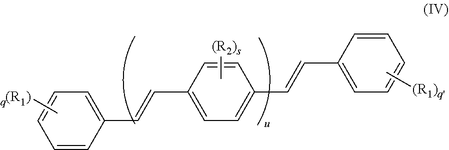

wherein
R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
R₂ is independently selected from halogen, cyano, optionally substituted alkyl, optionally substituted alkenyl or optionally substituted alkynyl;
u is an integer selected from 3 to 15;
each s is an integer independently selected from 0 to 4; and
q is an integer selected from 2 to 5;
q′ is an integer selected from 2 to 5; and
b) flowing the cell and/or a lipid vesicle through the flow cytometer.

In some embodiments, the cells and/or lipid vesicles are in suspension.

In some embodiments, the cells are adherent cells.

In some embodiments, the incubation period is about 1 min to about 12 days.

In some embodiments, the cell and/or lipid vesicle is flowed through the flow cytometer without a purification step.

In some embodiments, compound of Formula (I), sub-Formula (Ia-Ig) and/or Formula (IV) has a fluorescence excitation in the wavelength of about 300 nm to about 1000 nm.

In some embodiments, compound of Formula (I), sub-Formula (Ia-Ig) and/or Formula (IV) has a fluorescence emission in the wavelength of about 300 nm to about 2000 nm.

In some embodiments, the cell and/or lipid vesicle incubated with a compound of Formula (I), sub-Formula (Ia-Ig) and/or Formula (IV) has an emission intensity of more than about 2 times to about 500 times relative to a control sample of the compound of Formula (I), sub-Formula (Ia-Ig) and/or Formula (IV).

In some embodiments, the method further comprises a step of contacting the cell and/or lipid vesicle with another dye.

The present invention also provides a flow system for detecting and/or quantifying cells and/or lipid vesicles, comprising:
a) a compound of Formula (I), sub-Formula (Ia-Ig) and/or Formula (IV) or a salt or solvate thereof for labelling the cells and/or the lipid vesicles;
b) an inlet for introducing the labelled cells and/or lipid vesicles into the flow system; c) a detection means in fluid communication with the inlet for detecting a fluorescence emission from the labelled cells and/or lipid vesicles; and
d) optionally a counter means for quantifying the labelled cells and/or lipid vesicles.

### BRIEF DESCRIPTION OF THE DRAWINGS

Embodiments of the present invention will now be described, by way of non-limiting example, with reference to the drawings in which:

FIG. 1 shows examples of conjugated oligoelectrolytes of the present invention.

FIG. 2 shows flow cytometry measurements using a compound of Formula (I) and/or Formula (IV) to selectively label the Gram-positive bacteria from Gram-negative bacteria.

FIG. 3 shows flow cytometry measurements of compounds of Formula (I) and/or Formula (IV) (a-f) and unlabelled exosomes (h)

FIG. 4 shows flow cytometry measurements of exosomes labelled with compounds of Formula (I) and/or Formula (IV)

FIG. 5 shows TEM micrographs of exosomes (a), exosomes labelled with compounds of Formula (I) and/or Formula (IV) (b) and exosomes labelled with compounds of Formula (I) and/or Formula (IV) collected after FACS (c).

FIG. 6 shows flow cytometry measurements of RBCs labelled with compound of Formula (I) and/or Formula (IV).

FIG. 7 shows percentage of flow cytometry events occurring in the gated areas of RBCs labelled with compound of Formula (I) and/or Formula (IV) to unstained RBCs at various ratios.

FIG. 8 shows flow cytometry measurements of Hep-G2 cells labelled with compounds of Formula (I) and/or Formula (IV).

FIG. 9 shows percentage of flow cytometry events occurring in the gated areas of stained Hep-G2 with compound of Formula (I) and/or Formula (IV) to unstained Hep-G2 at various ratios.

FIG. 10 shows flow cytometry measurements of two distinct populations of A549 cells labelled with compound of Formula (I) and/or Formula (IV) and unlabelled cells.

FIG. 11 shows flow cytometry measurements of passages of A549 cells labelled with compounds of Formula (I) and/or Formula (IV).

FIG. 12 shows confocal micrographs of HepG2 cells after stained by 4 μM compound of Formula (I) and 4 μM commercially available membrane dye FM 4-64: (a) compound of Formula (I) channel, (b) FM 4-64 channel, (c) brightfield channel, (d) merged channel. The scale bars are 20 μm.

FIG. 13 shows fluorescence microscopy images of A549 cells were stained with compounds of Formula (I) and/or Formula (IV).

FIG. 14 shows (a-b) Photoluminescence (PL) spectra of SUV, compounds of Formula (I) and/or Formula (IV) and the combination of SUV and compounds in PBS buffer, (c) three compounds of Formula (I) and/or Formula (IV) with different emission peaks after addition of SUV in PBS.

FIG. 15 shows the different emission wavelengths of the compounds of Formula (I) and/or Formula (IV).

FIG. 16 shows dynamic light scattering size distribution curves by intensity for the liposome without (grey solid line) and labelled with (dark dash line) compound of Formula (I) and/or Formula (IV).

FIG. 17 shows particle size distribution plots of dye only controls and their corresponding gated dot plots.

FIG. 18 shows particle size distribution plots of COE-labelled SW480 exosomes (10 μM) and their corresponding gated dot plots.

FIG. 19 shows particle size distribution plots of dye-labeled SW480 exosomes (10 μM and 20 PM) after purifying excess dyes using ultracentrifugation and their corresponding gated dot plots.

FIG. 20 shows imaging flow cytometry images of A549 cells stained by the COE-Ben-stained exosomes for different treatment and times, and their corresponding flow cytometric analysis.

FIG. 21 shows colocalization micrographs of A549 cells after be incubated with 2 μM COE-BT, and then stained by Early or Late Endosomes-GFP reagent (BacMam 2.0) or 100 nM lysosome-specific dye LysoTracker® Green DND-26.

FIG. 22 shows transmission electron microscopy image of EVs secreted by COE-BT-stained A549 cells and flow cytometry analysis of EVs secreted by COE-BT-stained A549 cells after the first 24-hour incubation.

FIG. 23a-b shows photographs and absorption spectra of 50 μM COEs or DiR solutions in PBS before and after ultrafiltration.

FIG. 24 shows correlation coefficient curves of and DLS measured derived mean coutn rate of neat PBS, 1 μM COE-BT, 1 μM DiR, 1 mM SUVs in PBS or 1 μM other COEs in PBS as measured by dynamic light scattering (DLS).

FIG. 25 shows photographs of Tyndall effects of neat PBS or 10 μM COEs in PBS after being illuminated using a red laser pointer.

FIG. 26 shows photographs of 200 μL COE solutions and dye solutions in PBS in a 96-well microplate before and after statically setting for 16 hours at room temperature.

FIG. 27 shows flow cytometry measurements for mixture of COE-BT-stained and COE-Ben-stained SUVs (130 nm) in PBS with different mixing ratio and left incubated for 1 h and 24 h analyzed on Cytoflex.

FIG. 28 shows SUV population percentages from FIG. 27 in different gates after mixing for 1 or 24 hours.

FIG. 29 shows dot plot profiles of dye-positive events in which POPC liposomes of 100, 200, 400 and 800 nm size, labelled with 0.5 mol % COE-Ben and COE-BT were analyzed on the Cytoflex.

FIG. 30 shows confocal micrographs of 6.25 mg mL-1 LMVs (large multilamellar vesicles) after stained by 15 μM COE-Ben and 15 μM FM 4-64 in PBS for 30 minutes at room temperature, and gray value curves represent fluorescence intensity profile of the white line in its left-side fluorescent micrograph for both channels.

FIG. 31 shows photograph of COEs without (−) or with (+) 1 mM SUV treatment in PBS under UV-light (365 nm) exposure using a handheld UV lamp.

FIG. 32 shows zeta potential measurements for 1 mM POPC only SUVs stained by 5 μM of different COEs in DI water, and DLS measured Z-Average size and PDI for 1 mM POPC only SUVs stained by 5 μM of different COEs in DI water.

FIG. 33 shows confocal microscopy of fresh red blood cells (RBC) that were (a) unstained, (b) labeled with 2 μM COE-S6 and (c) 1 μg/mL Cell Mask Deep Red.

FIG. 34 shows confocal microscopy of red blood cells (RBC) that were stored for 22 days and (a) unstained, (b) labeled with 2 μM COE-S6 and (c) 1 μg/mL Cell Mask Deep Red.

FIG. 35 shows (a-c) FSC/SSC dot plots of red blood cells (RBC) measured on flow cytometry that are unstained, labelled with 1 μM COE-S6 and 0.5 μg mL-1 Cell Mask Deep Red, and (d) histograms showing the increase in fluorescence when RBCs were labelled with increasing COE-S6 concentrations and (e) the decrease in fluorescence when RBCs were labelled with increasing Cell Mask Deep Red due to quenching effect. The coefficient of variance (CV) of the histogram data is shown in (f).

FIG. 36 shows flow cytometry measurement of COE-BBT-stained A549 cells by excitation using 808 nm laser and collecting the emission using 950 nm long-pass filter. Unstained A549 cells were employed as negative control.

FIG. 37 shows cytotoxicity measurements against A549 cells for COEs.

FIG. 38 shows hemolysis measurements for COEs against bovine erythrocytes in PBS.

### DETAILED DESCRIPTION

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

“Alkynyl” refers to alkynyl groups preferably having from 2 to 10 carbon atoms and more preferably 2 to 6 carbon atoms and having at least 1, and preferably from 1-2, carbon to carbon, triple bonds. Examples of alkynyl groups include ethynyl (—C≡CH), propargyl (—CH₂C≡CH), pent-2-ynyl (—CH₂C≡CCH₂—CH₃), and the like.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkenyloxy” refers to the group alkenyl-O— wherein the alkenyl group is as described above.

“Alkynyloxy” refers to the group alkynyl-O— wherein the alkynyl groups is as described above.

“Halo” or “halogen” refers to fluoro, chloro, bromo and iodo.

“Acyl” refers to groups H—C(O)—, alkyl-C(O)—, cycloalkyl-C(O)—, aryl-C(O)—, heteroaryl-C(O)— and heterocyclyl-C(O)—, where alkyl, cycloalkyl, aryl, heteroaryl and heterocyclyl are as described herein.

“Oxyacyl” refers to groups HOC(O)—, alkyl-OC(O)—, cycloalkyl-OC(O)—, aryl-OC(O)—, heteroaryl-OC(O)—, and heterocyclyl-OC(O)—, where alkyl, cycloalkyl, aryl, heteroaryl and heterocyclyl are as described herein.

“Amino” refers to the group —NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminoacyl” refers to the group —C(O)NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Acylamino” refers to the group —NR″C(O)R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Acyloxy” refers to the groups —OC(O)-alkyl, —OC(O)-aryl, —C(O)O-heteroaryl, and —C(O)O-heterocyclyl where alkyl, aryl, heteroaryl and heterocyclyl are as described herein.

“Aminoacyloxy” refers to the groups —OC(O)NR″-alkyl, —OC(O)NR″-aryl,
—OC(O)NR″-heteroaryl, and —OC(O)NR″-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

‘Cyano’ refers to the group —CN.

“Oxyacylamino” refers to the groups —NR″C(O)O-alkyl, —NR″C(O)O-aryl,
—NR″C(O)O-heteroaryl, and NR″C(O)O-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxyacyloxy” refers to the groups —OC(O)O-alkyl, —O—C(O)O-aryl, —OC(O)O-heteroaryl, and —OC(O)O-heterocyclyl where alkyl, cycloalkyl, aryl, heteroaryl, and heterocyclyl are as described herein.

“Thio” refers to groups H—S—, alkyl-S—, cycloalkyl-S—, aryl-S—, heteroaryl-S—, and heterocyclyl-S—, where alkyl, cycloalkyl, aryl, heteroaryl and heterocyclyl are as described herein.

“Phosphoryl” refers to the groups —P(O)(R′″)(OR′″) where R′″ represents OR′″ or is hydroxyl, alkyl or amino and R′″ is alkyl, cycloalkyl, aryl or arylalkyl, where alkyl, amino, alkenyl, aryl, cycloalkyl, and arylalkyl are as described herein.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like.

“Heteroaryl” refers to a monovalent aromatic heterocyclic group which fulfils the Huckel criteria for aromaticity (ie. contains 4n+2 π electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (eg. pyridyl, pyrrolyl or N-oxides thereof or furyl) or multiple condensed rings (eg. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

“Arylene” refers to a divalent aryl group wherein the aryl group is as described above.

“Heteroarylene” refers to a divalent heteroaryl group wherein the aryl group is as described above.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroarylalkyl, heteroaryloxy, heterocyclyl, heterocyclylalkyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

“Hydrophilic” refers to molecules or moieties which have a greater affinity for, and thus solubility in, water as compared to organic solvents. For example, the hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound may be considered to be hydrophilic.

“Hydrophobic” refers to molecules or moieties which have a greater affinity for, and thus solubility in, organic solvents as compared to water. For example, the hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound may be considered to be hydrophobic.

Conjugated oligoelectrolytes (COEs) are a class of molecules defined by a hydrophobic conjugated core bearing terminal polar ionic pendants. In particular embodiments, the hydrophobic and hydrophilic moieties in COEs can be rationally designed into the molecule such that they mirror the organization of hydrophilic and hydrophobic domains in lipid bilayers. This structural design typically involves only unbranched internal structures with charged groups at the two termini so that it favours the spontaneous intercalation of COEs into cellular membranes, which is driven by electrostatic and hydrophobic interactions between the COEs and the lipids. The inventors have postulated that the fluorescence which originate from the π-π conjugation in the backbone can be tailored to occur at different wavelengths across the electromagnetic spectrum, including the UV, visible light, and infrared regions. This can be achieved by fine-tuning the optoelectronic properties of the conjugated core via structural derivation. For example, the backbone conjugated π system can be modulated based on a donor and acceptor moieties to achieve the range of emission wavelength from 300 to 2000 nm. Towards this end, the fluorescence excitation, emission and Stokes shift can be tuned. Further, COEs can be modified to exhibit selectivity over specific membranes through the functionalization of different chemical groups that control its membrane-intercalation abilities. This provides a target specific functionality.

Characteristically, the fluorescence emission of COEs enhance significantly upon their intercalation into lipid bilayers from the aqueous solution. This “light up” mechanism confers a high signal-to-noise ratio for COEs when they are localized within the more hydrophobic environment of lipid bilayers. Additionally, COEs have a distinct chemical structure from many commercially available lipophilic dyes, which usually contain a surfactant-like structure, i.e., one side of the molecule is hydrophobic, and the other side is hydrophilic. These surfactant-like structures will induce micelle-like aggregation in the aqueous solutions. For example, the commonly used membrane dye, PKH-26, has been shown to form aggregates, which have a similar size and fluorescence intensity compared to small particles such as the exosomes, thereby leading to false-positive signals. These phenomenona can be avoided in the case of COEs given that their emission has been shown to greatly intensify after intercalation into the lipid bilayer; i.e. high signal to noise ratio.

Accordingly, the present invention provides a compound of Formula (I) or a salt or solvate thereof:

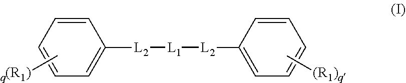

wherein
each R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
q is an integer selected from 1 to 5;
q′ is an integer selected from 1 to 5; wherein
each L₂ is independently selected from optionally substituted ethylene, or optionally substituted phenylethylene;
L₁ is a π-conjugated core comprising monomeric unit A and monomeric unit D:

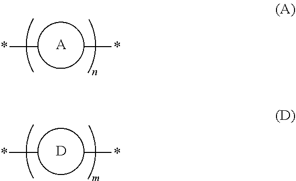

wherein
each A is independently selected from optionally substituted alkenylene, optionally substituted arylene or optionally substituted heteroarylene;
each D is independently selected from optionally substituted alkenylene, optionally substituted arylene or optionally substituted heteroarylene;
n is an integer selected from 1 to 5;
m is an integer selected from 1 to 5;
wherein * represents a bond to another monomeric unit or to L₂;
wherein monomeric units A and monomeric units D are alternatively bonded to each other;
wherein the compound of Formula (I) has a substantially linear topology.

In some embodiments, A and D are not both alkenylene. In other embodiments, A and D are not both phenylene. In other embodiments, A and D are not both alkenylene and phenylene. In some embodiments, L₁ is not butadienylene, polyalkenylene, phenylalkenylene and polyphenylalkenylene.

In some embodiments, the compound of Formula (I) or a salt or solvate thereof:

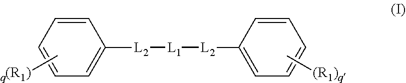

wherein
each R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
q is an integer selected from 1 to 5;
q′ is an integer selected from 1 to 5;
wherein
each L₂ is independently selected from optionally substituted ethylene, or optionally substituted phenylethylene;
L₁ is a π-conjugated core comprising monomeric unit A and monomeric unit D:

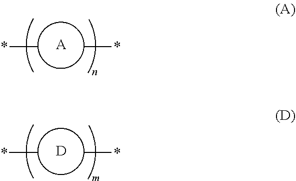

wherein
each A is independently selected from alkenylene substituted with cyano, optionally substituted monocyclic heteroarylene or optionally substituted fused heteroarylene;
each D is independently selected from alkenylene, phenylene, optionally substituted fused arylene, optionally substituted monocyclic heteroarylene or optionally substituted fused heteroarylene;
n is an integer selected from 1 to 5;
m is an integer selected from 1 to 5;
wherein * represents a bond to another monomeric unit or to L₂;
wherein monomeric units A and monomeric units D are alternatively bonded to each other;
wherein the compound of Formula (I) has a substantially linear topology.

A further advantage is that these compounds do not require a bioconjugation chemical reaction, such as the ones needed to label antibodies, in order to be used as a fluorescence label.

The compound of Formula (I) has a substantially linear topology. The topology refers to a molecular structure of a compound within the constraints of three-dimensional (3D) space. Such linear topology has two nodes as the termini without any junction nodes. The linear topology is advantageous for facilitating lipid membrane intercalation.

In some embodiments, the bonds connecting monomeric units A and monomeric units D in L₁ are substantially aligned along a longitudinal axis of the compound. In other embodiments, the monomeric units A and monomeric units D in L₁ are substantially aligned along a longitudinal axis of the compound. In this regard, bonds connecting monomeric units A and monomeric units D when offset from the longitudinal axis of the compound are within the scope of the invention.

The compounds of Formula (I) are linear in order to accommodate its position within the lipid bilayer. In some embodiments, the compounds are not branched; i.e. the monomeric units only extend along a single chain. In some embodiments, the compounds of Formula (I) are symmetrical in nature. The symmetry of a compound can be described by at least one of the 32 point groups. A Point Group describes all the symmetry operations that can be performed on a molecule that result in a conformation indistinguishable from the original. In this regard, in some embodiments, the compounds of Formula (I) have a C₂ᵥ point group.

In some embodiments, L₂ is independently selected from optionally substituted ethylene, or optionally substituted phenylethylene. In other embodiments, L₂ is independently selected from:

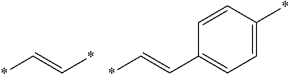

wherein * represents a bond to a monomeric unit and to a terminal phenyl moiety in compound of Formula (I).

L₁ is a π-conjugated core. A conjugated system is a system of connected p orbitals with delocalized electrons in a molecule, which in general lowers the overall energy of the molecule and increases stability. Lone pairs, radicals or carbenium ions may be part of the system, which may be cyclic, acyclic, linear or mixed.

In some embodiments, when L₁ comprises 6 membered aryl or heteroaryl, or when L₁ comprises fused aryl or heteroaryl having a 6 membered ring, the monomeric units are 1,4 conjugated on the 6 membered ring. In other embodiments, when L₁ comprises 5 membered aryl or heteroaryl, or when L₁ comprises fused aryl or heteroaryl having a 5 membered ring, the monomeric units are 1,4 conjugated or 2,5 conjugated on the 5 membered ring.

Alternatively, L₁ can be represented by at least one monomeric unit A and at least one monomeric unit D. In some embodiments, n and m in combination is an integer selected from 2 to 10, 3 to 10, 3 to 9, 3 to 8, 3 to 10, or 3 to 7.

In some embodiments, L₁ is selected from:

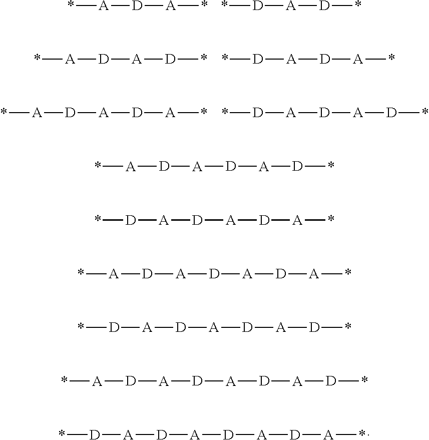

As mentioned, each A and D can be the same moiety such that L₁ is an alternating π-conjugated core. Alternatively, each A and D can be different. As L₁ comprises an alternating donor/acceptor composition of structural units (relative to each other), structures in which the alternating donor/acceptor composition is not adhered to are excluded from the scope of this invention. For example, butadienylene, polyalkenylene, phenylalkenylene and polyphenylalkenylene are excluded.

In some embodiments, the optional substituent on L₁ is selected from halogen, cyano, optionally substituted alkyl, optionally substituted alkenyl, optionally substituted alkynyl, optionally substituted alkoxy, optionally substituted alkenyloxy, and optionally substituted alkynyloxy. In other embodiments, the optional substituent on D is selected from halogen, cyano, alkyl, alkenyl, alkoxy, and alkenyloxy. In some embodiments, the optional substituent on L₁ is independently selected from halogen, cyano, optionally substituted alkyl, optionally substituted alkenyl or optionally substituted alkynyl.

As used herein, monomeric unit D is an electron donating (rich) moiety relative to monomeric unit A. In this regard, monomeric unit A is an electron withdrawing/accepting (poor) moiety. When in sequence the D-A combination gives rise to intramolecular charge transfer excited states with optical absorption and emission further into the red, as compared to a sequence of similar moieties (-Dₙ- or -Aₙ-) in conjugation.

In some embodiments, A is an electron accepting moiety. An electron acceptor is a chemical entity that accepts electrons transferred to it from another moiety or compound. In some embodiments, A has electron accepting substituents. In other embodiments, A has electron withdrawing substituents.

In some embodiments, A is independently selected from optionally substituted alkenylene or optionally substituted heteroarylene. In some embodiments, A is independently selected from cyano substituted alkenylene or optionally substituted heteroarylene. In some embodiments, the cyano substituted alkenylene is mono-substituted alkenylene or di-substituted alkenylene. In other embodiments, the optionally substituted heteroarylene is optionally substituted monocyclic heteroarylene or optionally substituted fused heteroarylene. The heteroarylene can be 5 membered heteroarylene or a 6 membered heteroarylene. The heteroarylene can be a fused heteroarylene. In some embodiments, the heteroarylene is a fused 5,5 membered heteroarylene, fused 5,6 membered heteroarylene, fused 6,6 membered heteroarylene, fused 5,5,6 membered heteroarylene, fused 5,6,6 membered heteroarylene, fused 6,6,6 membered heteroarylene, fused 5,5,6,6 membered heteroarylene, fused 5,6,6,6 membered heteroarylene, or fused 6,6,6,6 membered heteroarylene.

In some embodiments, the optional substituent on A is selected from halogen, cyano, optionally substituted alkyl, optionally substituted alkenyl, optionally substituted alkynyl, optionally substituted alkoxy, optionally substituted alkenyloxy, and optionally substituted alkynyloxy. In other embodiments, the optional substituent on A is selected from halogen, cyano, alkyl, alkenyl, alkoxy, and alkenyloxy.

In some embodiments, A is selected from

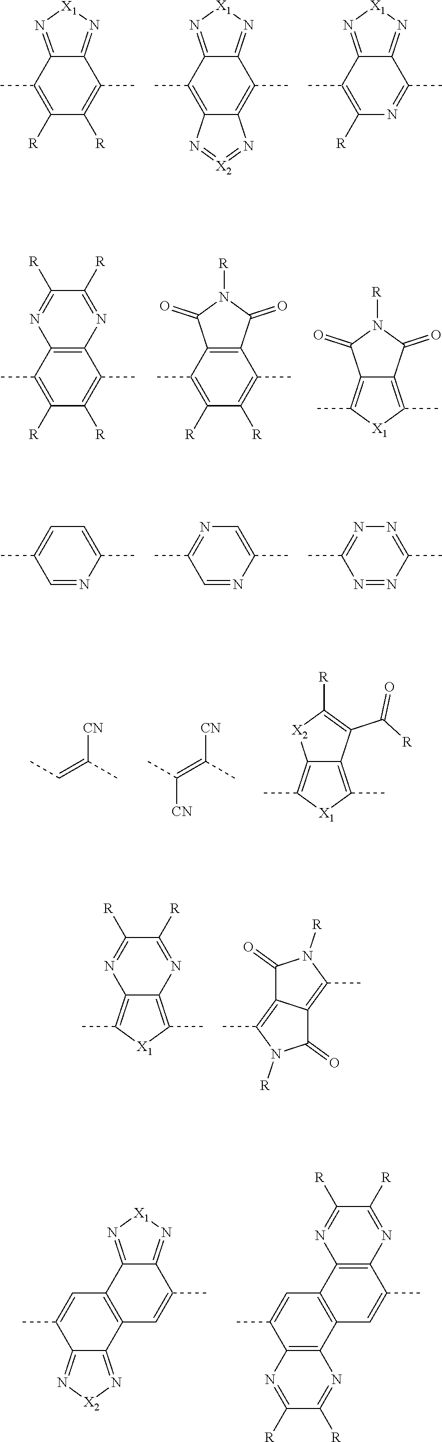

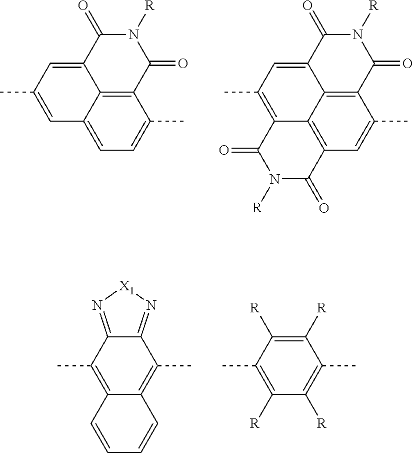

wherein [symbol] represents a bond to D or to L₂;
each X₁ is independently selected from C, O, N, S and Se;
each X₂ if present is independently selected from C, O, N, S and Se;
when X₂ is present, at least one of X₁ and X₂ is O, N or S;
R is independenly selected from H, halo, cyano, and optionally substituted alkyl.

In some embodiments, A is a moiety of Formula (II):

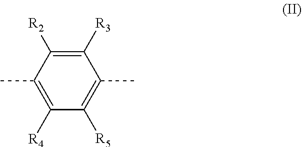

wherein [symbol] represents a bond to D or to L₂;
R₂, R₃, R₄ and R₅ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₂ and R₃ are linked to form optionally substituted heterocyclyl, optionally substituted heteroaryl; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl.

In some embodiments, R₂ and R₃ are independently selected from H, halogen, optionally substituted alkyl, optionally substituted alkenyl, optionally substituted alkynyl. In other embodiments, R₂ and R₃ are independently selected from H, halogen, optionally substituted alkyl, optionally substituted alkenyl. In other embodiments, R₂ and R₃ are independently selected from H, halogen, optionally substituted C₁-C₆ alkyl, optionally substituted C₂-C₆ alkenyl, optionally substituted C₁-C₆ alkoxy, and optionally substituted C₂-C₆ alkenyloxy. In other embodiments, R₂ and R₃ are independently selected from H, halogen, and C₁-C₆ alkyl.

In some embodiments, A is a moiety of Formula (II):

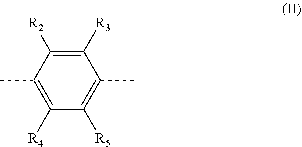

wherein [symbol] represents a bond to D or to L₂;
R₂ and R₃ are linked to form optionally substituted heterocyclyl, optionally substituted heteroaryl;
R₄ and R₅ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl.

In some embodiments, R₂ and R₃ are linked to form optionally substituted heteroaryl such that it forms a conjugated π system with the phenyl moiety.

In some embodiments, R₄ and R₅ are independently selected from H, halogen, optionally substituted alkyl, optionally substituted alkenyl, optionally substituted alkynyl. In other embodiments, R₄ and R₅ are independently selected from H, halogen, optionally substituted alkyl, optionally substituted alkenyl. In other embodiments, R₄ and R₅ are independently selected from H, halogen, optionally substituted C₁-C₆ alkyl, optionally substituted C₂-C₆ alkenyl, optionally substituted C₁-C₆ alkoxy, and optionally substituted C₂-C₆ alkenyloxy. In other embodiments, R₄ and R₅ are independently selected from H, halogen, and C₁-C₆ alkyl.

In some embodiments, R₄ and R₅ are linked to form optionally substituted cycloalkyl, optionally substituted cycloalkenyl, optionally substituted heterocyclyl, optionally substituted aryl, optionally substituted heteroaryl. In other embodiments, R₄ and R₅ are linked to form optionally substituted cycloalkyl, optionally substituted heterocyclyl, optionally substituted aryl, optionally substituted heteroaryl. In some embodiments, R₄ and R₅ are linked to form optionally substituted aryl, or optionally substituted heteroaryl. In some embodiments, R₄ and R₅ are linked to form optionally substituted heteroaryl such that it forms a conjugated π system with the phenyl moiety.

In some embodiments, A is

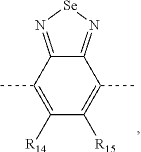

wherein R₄ and R₅ are as disclosed herein.

In some embodiments, A is

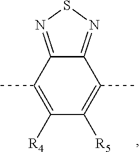

wherein R₄ and R₅ are as disclosed herein.

In some embodiments, A is

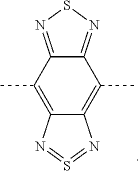

In some embodiments, n is an integer selected from 1 to 4, 1 to 3, 1 to 2, 2 to 4, 3 to 4 or 3 to 5.

In some embodiments, D is an electron donating moiety. An electron donor is a chemical entity that donates electrons transferred from it to another moiety or compound. In some embodiments, D has electron donating substituents.

In some embodiments, D is independently selected from optionally substituted alkenylene, optionally substituted arylene or optionally substituted heteroarylene. In some embodiments, D is independently selected from alkenylene, arylene or optionally substituted heteroarylene. In other embodiments, the arylene is phenylene. In other embodiments, the optionally substituted heteroarylene is optionally substituted monocyclic heteroarylene or optionally substituted fused heteroarylene. The heteroarylene can be 5 membered heteroarylene or a 6 membered heteroarylene. The heteroarylene can be a fused heteroarylene. In some embodiments, the heteroarylene is a fused 5,5 membered heteroarylene, fused 5,6 membered heteroarylene, fused 6,6 membered heteroarylene, fused 5,5,6 membered heteroarylene, fused 5,6,6 membered heteroarylene, fused 6,6,6 membered heteroarylene, fused 5,5,6,6 membered heteroarylene, fused 5,6,6,6 membered heteroarylene, or fused 6,6,6,6 membered heteroarylene.

In some embodiments, the optional substituent on D is selected from halogen, cyano, optionally substituted alkyl, optionally substituted alkenyl, optionally substituted alkynyl, optionally substituted alkoxy, optionally substituted alkenyloxy, and optionally substituted alkynyloxy. In other embodiments, the optional substituent on D is selected from halogen, cyano, alkyl, alkenyl, alkoxy, and alkenyloxy.

In some embodiments, D is a moiety selected from

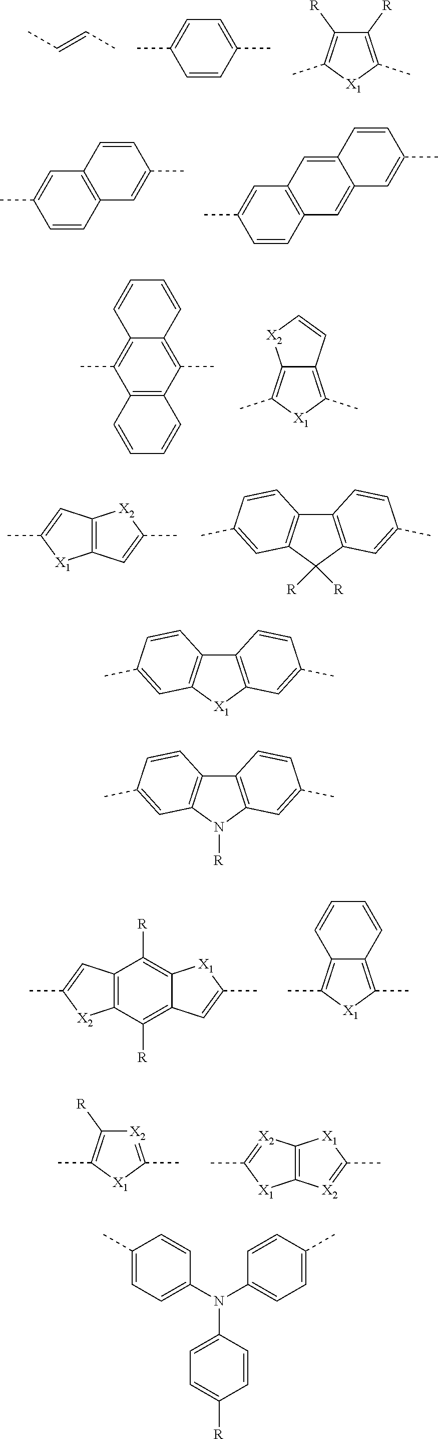

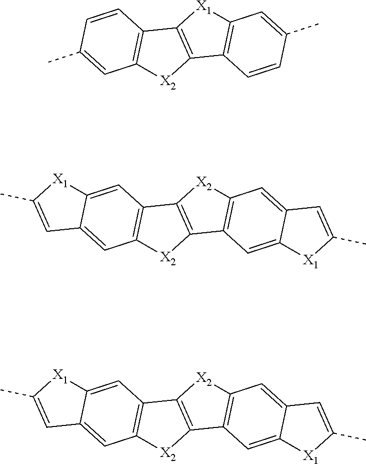

wherein [symbol] represents a bond to A or to L₂;
each X₁ is independently selected from C, O, N, S, and Se;
each X₂ if present is independently selected from C, O, N, S and Se;
when X₂ is present, at least one of X₁ and X₂ is O, N or S;
R is independently selected from H, halo, cyano, and optionally substituted alkyl.

In some embodiments, D is an optionally substituted 5 membered heteroarylene.

In some embodiments, D is a moiety of Formula (III):

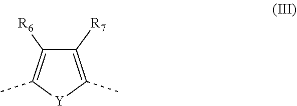

wherein Y is NR, O, S, or Se;
R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl; and
R is selected from H, halo, cyano, and optionally substituted alkyl.

In some embodiments, R₆ and R₇ are independently selected from H, halogen, optionally substituted alkyl, optionally substituted alkenyl, optionally substituted alkoxy, or optionally substituted alkenyloxy.

In other embodiments, R₆ and R₇ are independently selected from H, halogen, optionally substituted alkyl, or optionally substituted alkoxy. In other embodiments, R₆ and R₇ are independently selected from H, halogen, optionally substituted C₁-C₆ alkyl, optionally substituted C₂-C₆ alkenyl, optionally substituted C₁-C₆ alkoxy, or optionally substituted C₂-C₆ alkenyloxy. In other embodiments, R₆ and R₇ are independently selected from H, halogen, optionally substituted C₁-C₆ alkyl, or optionally substituted C₁-C₆ alkoxy. In other embodiments, R₆ and R₇ are independently selected from H, halogen, or optionally substituted C₁-C₆ alkyl.

In some embodiments, R₆ and R₇ are linked to form optionally substituted cycloalkyl, optionally substituted cycloalkenyl, optionally substituted heterocyclyl, optionally substituted aryl, optionally substituted heteroaryl. In other embodiments, R₆ and R₇ are linked to form optionally substituted cycloalkyl, optionally substituted heterocyclyl, optionally substituted aryl, or optionally substituted heteroaryl.

In some embodiments, D is

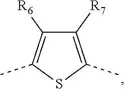

wherein R₆ and R₇ are as disclosed herein.

In some embodiments, D is

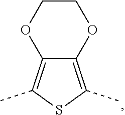

In some embodiments, m is an integer selected from 1 to 4, 1 to 3, 1 to 2, 2 to 4, 3 to 4, or 3 to 5.

In some embodiments, n and m together is at least 3. In other embodiments, n and m together is at least 4 or 5.

In some embodiments, each R₁ is independently selected from optionally substituted alkyl, optionally substituted alkoxy, optionally substituted acyl, optionally substituted oxyacyl, optionally substituted acyloxy, optionally substituted amino, optionally substituted aminoacyl, optionally substituted acylamino, optionally substituted oxyacyloxy or optionally substituted thio or optionally substituted phosphoryl. In other embodiments, R₁ is optionally substituted alkoxy, optionally substituted oxyacyl or optionally substituted amino. In another embodiment, R₁ is optionally substituted polyethoxy, wherein the monomeric unit is from 3 to 10. In other embodiments, the chain length of R₁ is from about 3 to about 10. In other embodiments, R₁ is optionally substituted C₃-C₁₀ alkoxy, optionally substituted C₃-C₁₀ alkylamino, optionally substituted C₃-C₁₀ dialkylamino, optionally substituted C₃-C₁₀ alkyloxyacyl or optionally substituted polyethoxy. In some embodiments, R₁ is independently selected from optionally substituted alkyl, optionally substituted alkoxy. In some embodiments, R₁ is independently selected from alkyl and alkoxy, each optionally substituted with amino, or alkylamino.

In some embodiments, the optional substituent at R₁ is independently selected from oxy, oxyacyl, acyl, amino, phosphoryl, thiol, alkyl, alkenyl, alkynyl, oxyalkyl, alkylacyloxy, sulfonyl, chlorate or its charged species thereof. In some embodiments, the optional substituent at R₁ is independently selected from hydroxyl, carboxyl, phosphate, amino, alkylamino, dialkylamino, chlorate, sulphate, acetate or its charged species thereof. In some embodiments, the optional substituent at R₁ is a tertiary amino. The tertiary amino may be neutralised by a counterion, which can be a halide.

In other embodiments, the optional substituent at R₁ is a hydrophilic moiety. In other embodiments, the optional substituent at R₁ is a charged moiety. Examples of hydrophilic and/or charged moieties are trialkylammonium halide. For example, the charged moiety can be trimethylammonium iodide. In this embodiment, R₁ terminates with trimethylammonium, and thereby imparts a positive charge when substituted to R₁ (for example, alkyl). Other cationic charged groups include but are not limited to pyridinium, pyrrolidinium, imidazolium, guanidinium, sulfonium, thiouronium, and phosphonium.

Other anionic charged groups include but not limited to chlorate, sulphate, phosphate, acetate, carboxyl, hydroxide. The hydrophilic and/or charged moieties can also in zwitterionic form that contains both cationic and anionic charged groups through covalent bonds. The excess charges can be neutralized by acceptable cations or anions.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others. Examples of the optional substituents on R₁ can be selected from:

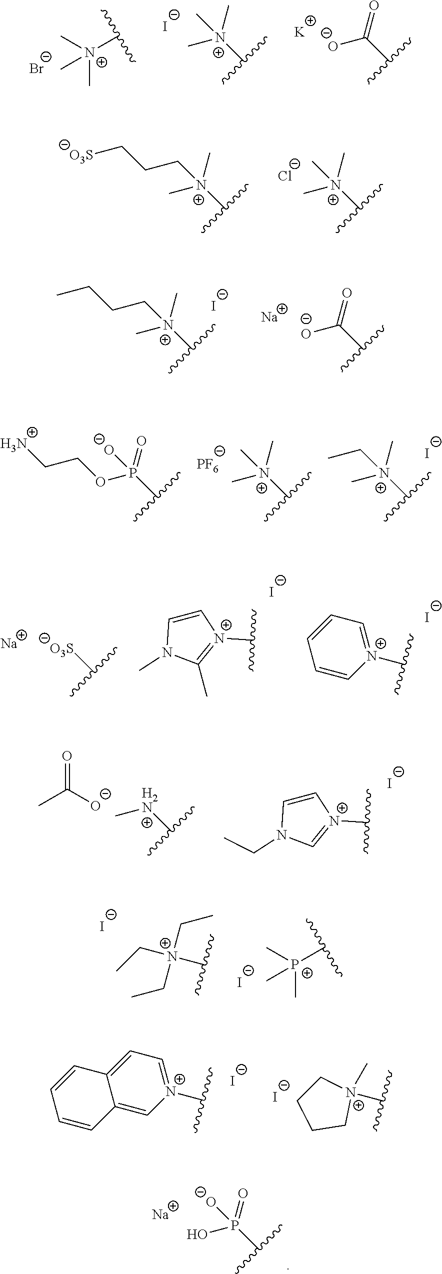

For example, to achieve the ‘Gram’ selectivity functionality, compound of the present invention can comprise charged ammonium groups. One side is needed to cross the hydrophobic bilayer core to achieve a membrane spanning configuration. Once this is achieved, the charged moieties ‘holds’ the compound across the bilayer such that it does not escape easily. This allows for the visualisation of Gram-positive bacterial cells for over long periods of time.

For compound of Formula (I) to have a balance of hydrophilic and hydrophobic properties to facilitate its intercalation within the lipid bilayer, at least one side chain should be present at each terminus of the backbone. In this regard, in some embodiments, q is an integer from 1 to 4. In other embodiments, q is an integer from 1 to 3. In some embodiments, q′ is an integer from 1 to 4. In other embodiments, q′ is an integer from 1 to 3. In this regard, there are on compound of Formula (I) or sub-Formulae (Ia-Ig), a total of at least 2 R₁ groups, at least 3 R₁ groups, at least 4 R₁ groups, at least 5 R₁ groups or at least 6 R₁ groups.

For compound of Formula (I) to maintain its linear configuration, R₁ are preferentially positioned at the meta and/or para positions of the terminus phenyl groups. In some embodiments, R₁ is present at the meta and para positions of the terminus phenyl groups. In other embodiments, R₁ is present at the meta or para positions of the terminus phenyl groups.

In some embodiments, the compound of formula (I) is a compound of Formula (Ia):

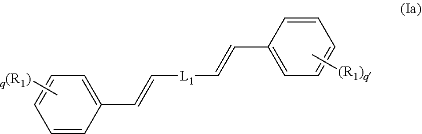

wherein L₁, A, D, R₁, n, m, q and q′ are as disclosed herein.

In some embodiments, the compound of formula (I) is a compound of Formula (Ib):

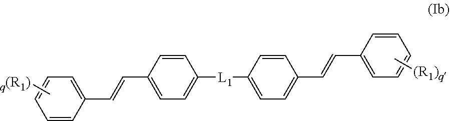

wherein L₁, A, D, R₁, n, m, q and q′ are as disclosed herein.

In some embodiments, the compound of formula (I) is a compound of Formula (Ib), wherein A is a moiety of Formula (II):

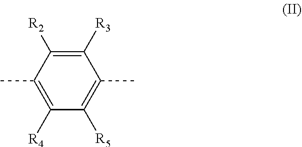

wherein [symbol] represents a bond to D or to L₂;
R₂ and R₃ are linked to form optionally substituted heterocyclyl, optionally substituted heteroaryl;
R₄ and R₅ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl; and
D is a moiety of Formula (III):

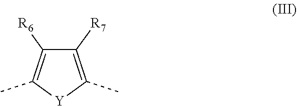

wherein Y is NR, O, or S;
R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl; and
R is selected from H, halo, cyano, and optionally substituted alkyl.

In some embodiments, the compound of formula (I) is a compound of Formula (Ib), wherein
A is

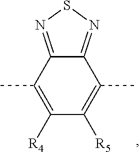

wherein R₄ and R₅ are as disclosed herein; and
D is

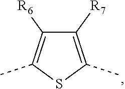

wherein R₆ and R₇ are as disclosed herein.

In some embodiments, the compound of Formula (I) is a compound of Formula (Ic):

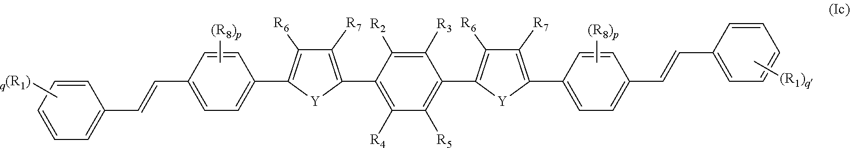

each R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
R₂ and R₃ are linked to form optionally substituted heterocyclyl, optionally substituted heteroaryl;
R₄ and R₅ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl;
R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl;
each R₈ is independently selected from halogen, cyano, optionally substituted alkyl, optionally substituted alkenyl or optionally substituted alkynyl;
Y is NR, O, or S;
each p is an integer independently selected from 0 to 4;
q is an integer selected from 1 to 5; and
q′ is an integer selected from 1 to 5.

In some embodiments, the compound of Formula (I) is a compound of Formula (Id):

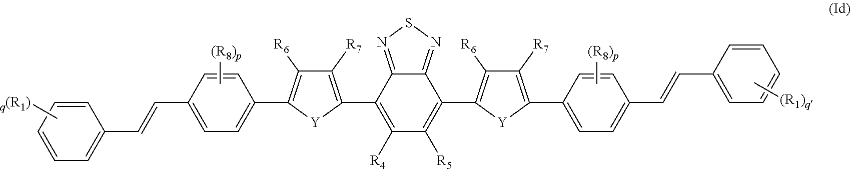

wherein R₁, R₄, R₅, R₆, R₇, R₈, Y, p, q and q′ are as disclosed herein.

In some embodiments, the compound of Formula (I) is a compound of Formula (Ie):

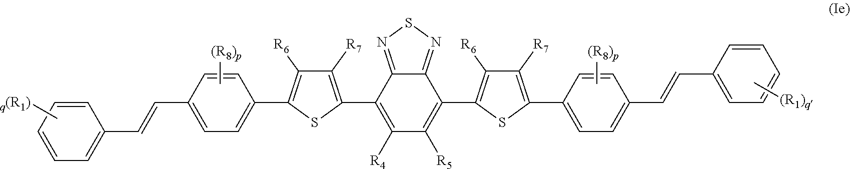

wherein R₁, R₄, R₅, R₆, R₇, R₈, p, q and q′ are as disclosed herein.

In some embodiments, the compound of Formula (I) is a compound of Formula (If):

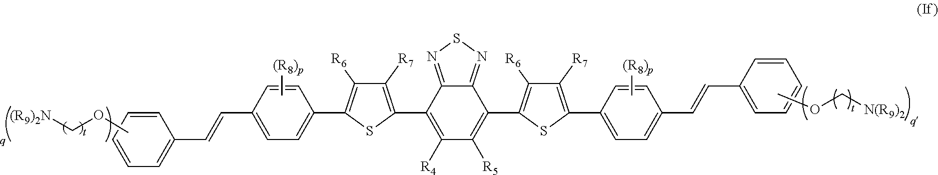

wherein R₁, R₄, R₅, R₆, R₇, R₈, p, q and q′ are as disclosed herein;
each R₉ is independently H or optionally substituted alkyl; and
each t is an integer independently selected from 1 to 8.

In some embodiments, each R₈ is independently selected from halogen, cyano, optionally substituted alkyl, optionally substituted alkenyl or optionally substituted alkynyl. In other embodiments, each R₈ is independently selected from halogen, cyano or optionally substituted alkyl. In other embodiments, each R₈ is independently selected from halogen, cyano, methyl, ethyl or propyl.

In some embodiments, each R₈ is independently selected H or optionally substituted C₁-C₅ alkyl. In other embodiments, each R₈ is independently selected H or C₁-C₅ alkyl. In other embodiments, each R₈ is independently selected from H, methyl, ethyl, propyl, isopropyl, cyclopropyl, butyl, sec-butyl, isobutyl or tertbutyl.

In some embodiments, each R₉ is optionally substituted alkyl. In other embodiments, each R₉ is C₁-C₅ alkyl. In other embodiments, each R₉ is independently selected from methyl, ethyl, propyl, isopropyl, cyclopropyl, butyl, sec-butyl, isobutyl or tertbutyl.

In some embodiments, the compound of Formula (I) or a salt or solvate thereof is selected from

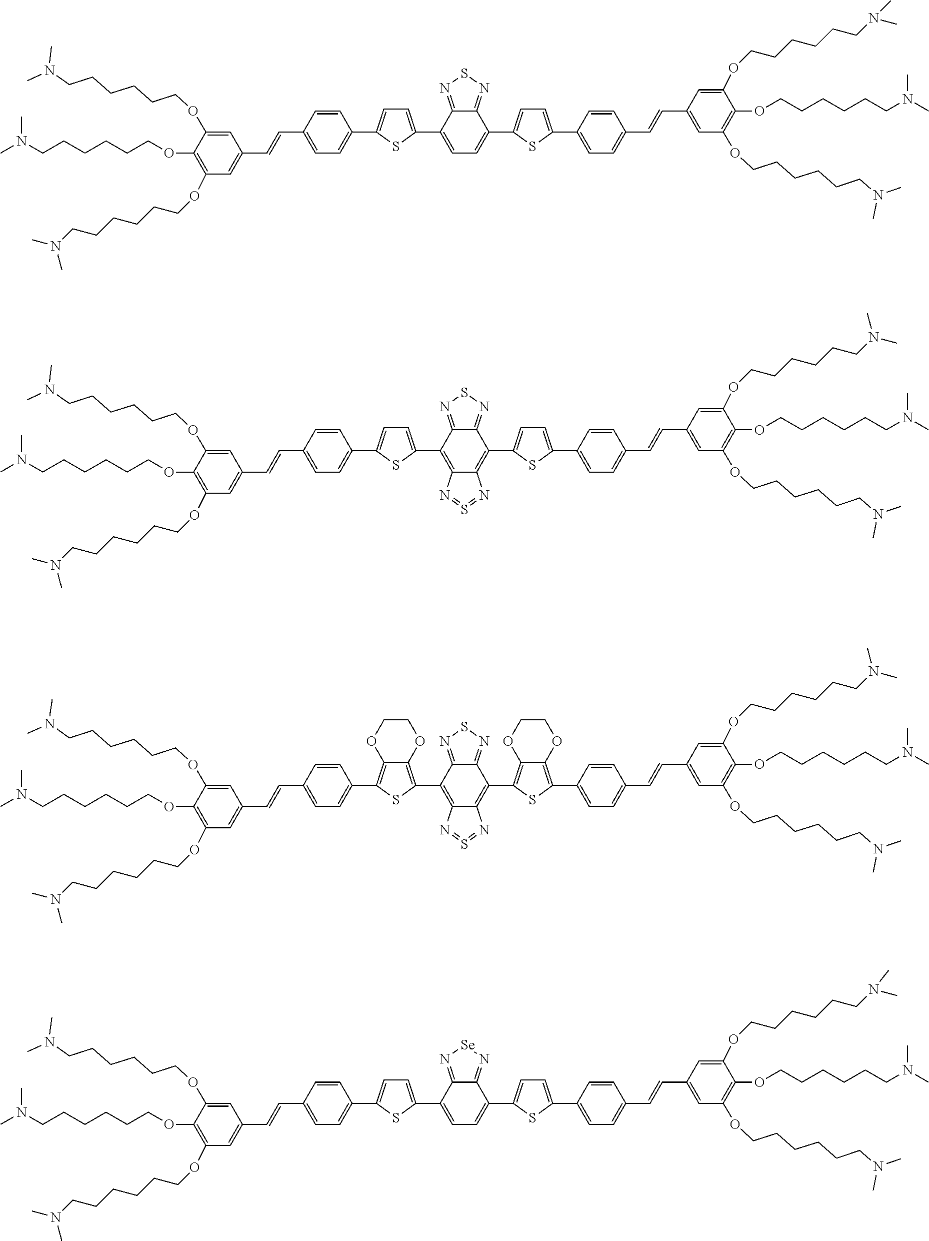

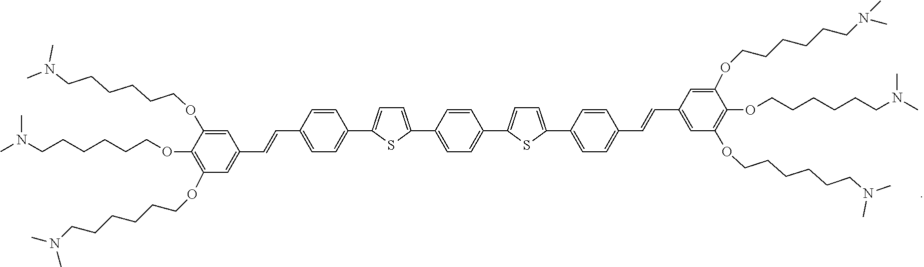

In some embodiments, the compound of Formula (I) is a salt thereof. The salt form can be a protonated salt, or can be generated by alkylating compound of Formula (I) with halocarbons. For example alkylhalide (such as CH₃Br or CH₃I) can be used. In some embodiments, the compound of Formula (I) or a salt or solvate thereof is a quaternary ammonium salt. In this regard, when R₁ is optionally substituted amino, each of R₁ can be alkylated to provide at least a positive charge at their respective ends.

For example, quaternary ammonium salts of compound of Formula (I) can be:

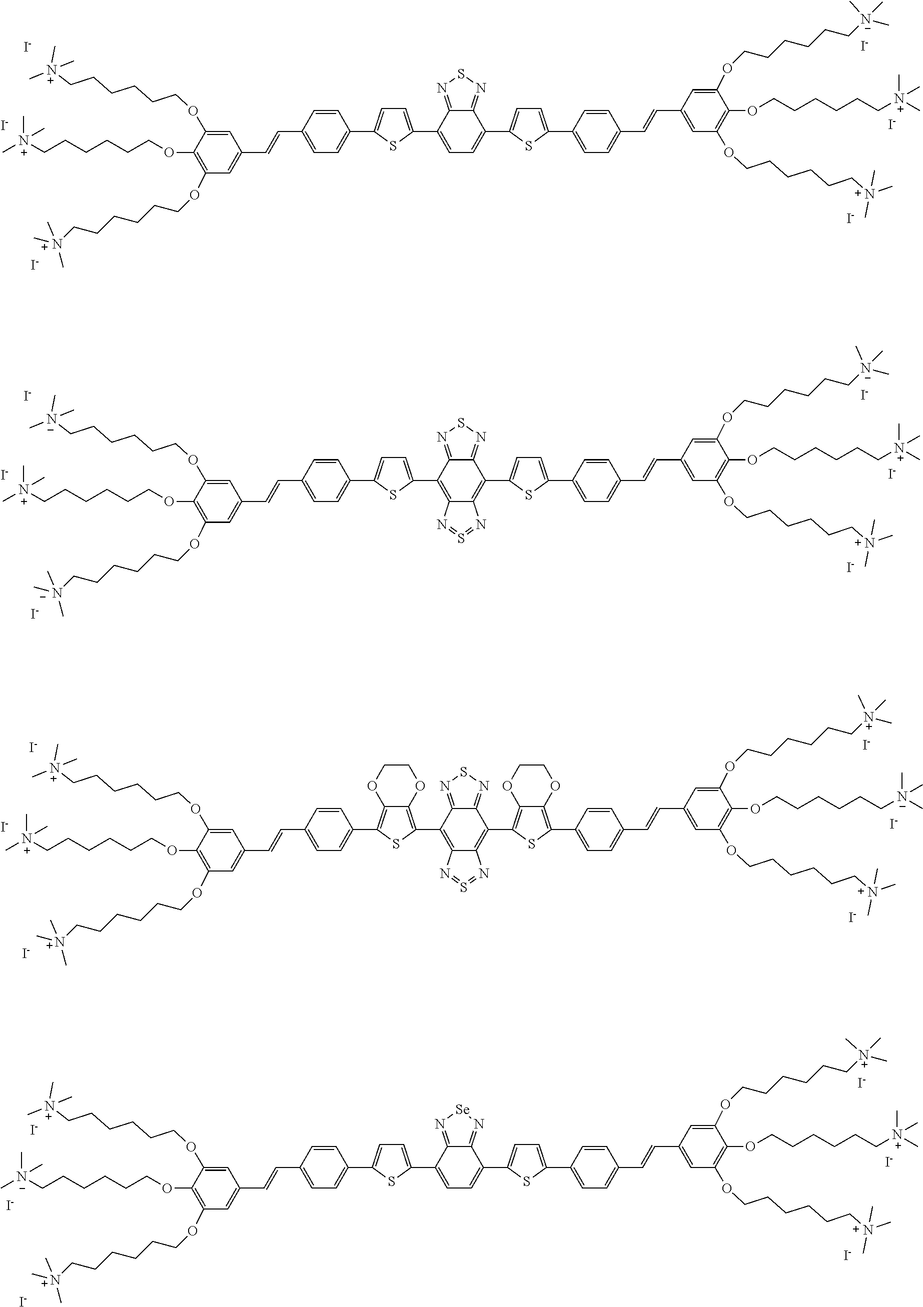

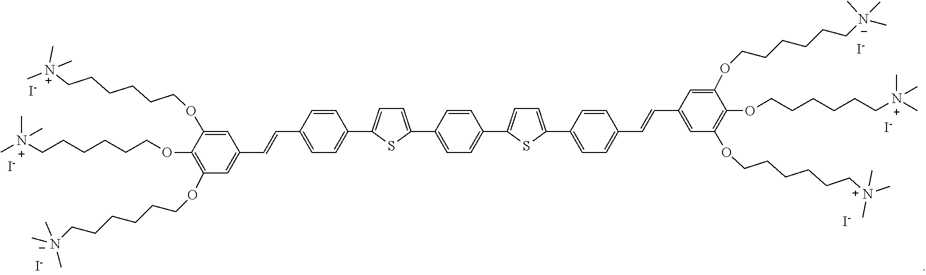

The compound of the invention may be in crystalline form either as the free compound or as a solvate (e.g. hydrate) and it is intended that both forms are within the scope of the present invention. Methods of solvation are generally known within the art.

The compounds of the present invention can be provided as a solid or as a solution. For example, the compound can be provided as a lyophilised powder.

The compounds of the present invention can be provided as a composition. The composition can comprise the compound in a polar medium as a single entity. As used herein, ‘polar medium’ includes polar protic and polar aprotic solvents. Polar solvents have large dipole moments or partial charges and contain bonds between atoms with very different electronegativities such as oxygen and hydrogen. Protic solvents have O—H or N—H bonds. Such bonds allow for participation in hydrogen bonding. Additionally, these O—H or N—H bonds can serve as a source of protons (H⁺). Aprotic solvents may have hydrogens on them somewhere, but they lack O—H or N—H bonds, and therefore cannot hydrogen bond with themselves. Polar solvents include, but is not limited to, dichloromethane, tetrahydrofuran, ethyl acetate, acetone, N,N-dimethylformamide, acetonitrile, dimethylsulfoxide, ammonia, butanol, propanol, ethanol, methanol, acetic acid and water. Included within this definition are also solvent mixtures, wherein the major component of the solvent mixture is a polar solvent. For example, water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also falls within this definition.

In some embodiments, the composition comprises a compound of Formula (I) or sub-Formulae (Ia-Ig) and a polar medium. For example, when the MIC value is 256 μM, in other embodiments, the composition comprises a compound of Formula (I) or sub-Formulae (Ia-Ig) and a polar medium, wherein the final concentration of the compound of Formula (I) or sub-Formulae (Ia-Ig) is about 130 μM. In other embodiments, the concentration is about 10 μM, about 20 μM, about 30 μM, about 40 μM, about 50 μM, about 100 μM, about 150 μM, about 200 μM, about 250 μM, about 300 μM, about 350 μM, or about 400 μM. In other embodiments, the concentration is not more than 10 μM, not more than 20 μM, not more than 30 μM, not more than 40 μM, not more than 50 μM, not more than 100 μM, not more than 150 μM, not more than 200 μM, not more than 250 μM, not more than 300 μM, not more than 350 μM, or not more than 400 μM.

Alternatively, the compounds can be provided as a kit. The kit can comprise the compound and the polar medium. The compound and the polar medium can be in separate vessels or as separately packaged components, to be mixed before use. Alternatively, the kit can comprise a composition of the compound in a first polar medium and separately a second medium, both components contained in separate vessels. The kit can additionally comprise another dye for staining a separate component of the bacterial cell. For example, the kit can additionally comprise FM 4-64. The kit can additionally comprise an excipient. The excipient can act to further stabilise the compound, and/or reduce the background noise by further quenching the fluorescence of the compound before its penetration into the bacterial cell membrane.

Flow cytometry is a technique to study biological cells, bacterial cells and extracellular vesicles (e.g., lipid vesicles, exosomes) in the research labs across both academia and industrial settings. These biological samples are defined by the essentiality of a lipid bilayer (membrane), of which COEs are designed to maintain a high affinity with. Hence, the physicochemical and optoelectronic properties of the compounds of the present invention when associated within a lipid bilayer allows them to be used a dye for flow cytometry applications.

Compounds of the present invention are suitable for use as a fluorescence probe. For example, the backbone of the compound can be tuned such that it has a certain length (≈3.4 nm), and further with suitable topology for specific membrane intercalation. The elongated backbone length can match the thickness of lipid bilayer (≈4 nm), thus can have negligibly toxic to cells and bacteria like E. coli and S. aureus (an MIC value greater than 256 μM). In this regard, the compounds can be used as a dye in a living system without compromised the cellular viability. Second, six positively charged side chains can be included into the compounds as terminal groups to achieve sufficient solubility in aqueous media. It was found that when the length of hydrophobic conjugated core increases, the number of charged side chains must be balanced to provide good aqueous solubility for COE compounds. The unfavorable hydrophilic/hydrophobic profile and strong aggregating tendency will affect the effective membrane intercalation and fluorescence stability (susceptible to fluorescence quenching). For example, a 5 benzene rings oligophenylenevinylene backbone with 4 positively charged side chains in both terminals shows some tendency to aggregate. Cloudy aqueous solution will be seen by naked eyes when it is at the concentration of 1 mg mL⁻¹, suggesting there are lots of aggregates in the incompletely dissolved suspension. When decorated by 6 charged side chains, the COE compounds exhibit excellent solubility in aqueous solution (>50 mg mL⁻¹). The clear and uniform aqueous solution feature of COE compounds add to its practical value, especially when it is needed to store at high concentration before dilution and use. Third, the elongated backbone of COE compounds is expected to favor greater membrane stability, while the enriched hydrophilic groups are expected to increase aqueous solubility. Besides, these combined physical features were intended to increase both hydrophobic and electrostatic interactions to promote intercalation within the bilayer. When the COE compounds is used as membrane labelling dye, it thus can stably incorporate within the lipid bilayers via the strong binding forces for a long-time staining.

FIG. 16 shows the variations in fluorescence emission as the A and D units of the compounds are varied. Taking reference from COE-Quin, in which alternating phenyl moieties are weakly electron accepting and electron donating, by modulating the electron accepting and donating properties of the A and D units, the fluorescence excitation and hence emission can be calibrated to a specific wavelength. The selection of these units also allows for a narrow FWHM and hence greater specificity.

Accordingly, the present invention also provides a compound of Formula (Ig) or a salt or solvate thereof:

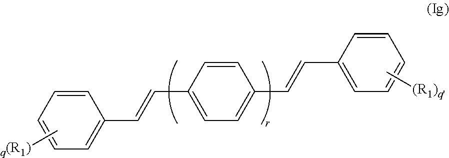

wherein
each R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
q is an integer selected from 1 to 5;
q′ is an integer selected from 1 to 5; and
r is an integer selected from 1 to 5.

The application of these compounds can be used in the staining of bacterial cells, mammalian (including but not limited to A549 cancer cells and red blood cells), and exosomes (especially unbound exosomes).

Accordingly, the present invention provides a method of labelling a cell and/or a lipid vesicle, comprising:
a) incubating a compound of Formula (I), sub-Formula (Ia-Ig) or a salt or solvate thereof with the cell and/or lipid vesicle.

The cell can be a mammalian cell or a bacterial cell. In some embodiments, the bacterial cell is a Gram-positive or Gram-negative bacterial cell. In other embodiments, the Gram-negative or Gram-positive bacterial cells is selected from the group consisting of E. coli, P. aeruginosa, S. aureus, E. faecalis, S. oneidensis, B. megaterium or a combination thereof. The mammalian cell can be from a cell line, or from a sample derived from a subject.

In some embodiments, the sample of bacterial cells is a sample of planktonic bacterial cells.

‘Planktonic bacterial cells’ as used herein refers to free flowing bacterial cells in suspension. This is as opposed to the sessile state (or biofilm), in which a structured community of bacterial cells is enclosed in a self-produced polymeric matrix and adherent to an inert or living surface. In this regard, planktonic bacteria are free-living bacteria and makes up the populations that grow in test tubes and flask cultures in the laboratory.

The lipid vesicle is a structure within or outside a cell, consisting of a liquid or cytoplasm enclosed by a lipid bilayer. Vesicles form naturally during the processes of secretion (exocytosis), uptake (endocytosis) and transport of materials within the plasma membrane. Alternatively, they may be prepared artificially, in which case they are called liposomes. Unilamellar lipid vesicles has one phospholipid bilayer, while multilamellar lipid vesicles has more than one bilayer. Vesicles can also fuse with other organelles within the cell. A vesicle released from the cell is an extracellular vesicle. For example, the lipid vesicle can be an exosome. Exosomes are membrane-bound extracellular vesicles that are produced in the endosomal compartment of most eukaryotic cells. These lipid vesicles are included within the scope.

In some embodiments, the lipid vesicle is an extracellular vesicle. In other embodiments, the lipid vesicle is an exosome.

In some embodiments, the method comprises:
contacting a cell and/or lipid vesicle sample under a predefined condition with a compound of Formula (I), sub-Formula (Ia-Ig) or a salt or solvate thereof.

In some embodiments, the cell and/or lipid vesicle is incubated with compound of Formula (I), sub-Formula (Ia-Ig) or a salt or solvate thereof at a compound concentration that is below its MIC value. In other embodiments, the cell and/or lipid vesicle is incubated with compound of Formula (I), sub-Formula (Ia-Ig) or a salt or solvate thereof at a compound concentration that is less than half its MIC value. In some embodiments, the cell and/or lipid vesicle is incubated with compound of Formula (I), sub-Formula (Ia-Ig) or a salt or solvate thereof at a compound concentration that is less than a third its MIC value. The minimum inhibitory concentration (MIC) is the lowest concentration of a chemical, which prevents visible growth of a bacterium or bacteria. MIC depends on the microorganism and the chemical. The MIC of a compound may be determined by incubating the cells in liquid media or on plates of solid growth medium (e.g. agar) with various concentrations of the compound and identifying the compound concentrations in which no bacteria grew and the next lower dose which allowed bacterial growth. This information can also be derived from a plot turbidity against compound concentration.

Other methods of determining the MIC value are available, such as Etest or Kirby-Bauer test can also be used.

In some embodiments, the MIC value is more than about 1 μM. In other embodiments, the MIC value is more than about 5 μM, about 10 μM, about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, or about 500 μM.

In some embodiments, the compound of Formula (I), sub-Formula (Ia-Ig) or a salt or solvate thereof is provided at a concentration of about 1 nM to about 100 μM. In other embodiments, the concentration is about 1 nM to about 90 μM, about 1 nM to about 80 μM, about 1 nM to about 70 μM, about 1 nM to about 60 μM, about 1 nM to about 50 μM, about 1 nM to about 40 μM, about 1 nM to about 30 μM, about 1 nM to about 20 μM, about 1 nM to about 10 μM, about 1 nM to about 5 μM, about 1 nM to about 1 μM, about 1 nM to about 900 nM, about 1 nM to about 800 nM, about 1 nM to about 700 nM, about 1 nM to about 600 nM, about 1 nM to about 500 nM, about 1 nM to about 400 nM, about 1 nM to about 300 nM, about 1 nM to about 200 nM, about 1 nM to about 100 nM, about 1 nM to about 50 nM, or about 1 nM to about 20 nM.

In some embodiments, the compound of Formula (I), sub-Formula (Ia-Ig) or a salt or solvate thereof with the cell and/or lipid vesicle are incubated at about 5° C. to about 50° C. In other embodiments, the temperature is about 5° C. to about 45° C., about 5° C. to about 40° C., about 5° C. to about 35° C., about 10° C. to about 35° C., about 15° C. to about 35° C., or about 15° C. to about 30° C. In other embodiments, the temperature is room temperature or ambient temperature.

In some embodiments, the compound of Formula (I), sub-Formula (Ia-Ig) or a salt or solvate thereof with the cell and/or lipid vesicle are incubated for about 5 min to about 120 min. In other embodiments, the duration is about 5 min to about 110 min, about 5 min to about 100 min, about 5 min to about 90 min, about 5 min to about 80 min, about 5 min to about 70 min, about 5 min to about 60 min, about 5 min to about 50 min, about 5 min to about 40 min, about 5 min to about 30 min, about 5 min to about 20 min, or about 5 min to about 10 min.

The compound of Formula (I), sub-Formula (Ia-Ig) or a salt or solvate thereof and the cell and/or lipid vesicle can be incubated in an aqueous medium.

The term ‘aqueous medium’ used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also falls within this definition.

The present invention also provides a method of detecting a cell and/or a lipid vesicle using a fluorescence detector, comprising:
a) incubating a compound of Formula (I), sub-Formula (Ia-Ig) or a salt or solvate thereof with the cell and/or a lipid vesicle; and
b) passing the cell and/or a lipid vesicle through the fluorescence detector.

As the compound of Formula (I), sub-Formula (Ia-Ig) or a salt or solvate thereof emits fluorescence when intercalated with the cell membrane or lipid bilayer, fluorescence based techniques can be used to detect cells and/or lipid vesicles incorporated with compound of Formula (I), sub-Formula (Ia-Ig) or a salt or solvate thereof. Examples of fluorescence based techniques include, but is not limited to, fluorescence microscopy, confocal microscopy, plate readers, fluorometer, fluorescence spectroscopy, and flow cytometry (such as fluorescence activated cell sorting).

In some embodiments, the method of detecting a cell and/or a lipid vesicle using a flow cytometer, comprises:
a) incubating a compound of Formula (I), sub-Formula (Ia-Ig) or a salt or solvate thereof with the cell and/or a lipid vesicle; and
b) flowing the cell and/or a lipid vesicle through the flow cytometer.

The cell and/or lipid vesicle when incorporated with compound of Formula (I), sub-Formula (Ia-Ig) or a salt or solvate thereof can be excited by an electromagnetic radiation (source) in a wavelength range of about 300 nm to about 1000 nm.

The present invention also provides a method of detecting a cell and/or a lipid vesicle using a flow cytometer, comprising:
a) incubating a compound of Formula (IV) or a salt or solvate thereof with the cell and/or a lipid vesicle;

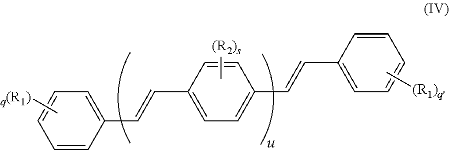

wherein
R₁ is independently selected from halogen, optionally substituted alkyl, optionally substituted alkenyl, optionally substituted alkynyl, optionally substituted alkoxy, optionally substituted alkenyloxy, optionally substituted alkynyloxy, optionally substituted acyl, optionally substituted oxyacyl, optionally substituted acyloxy, optionally substituted amino, optionally substituted aminoacyl, optionally substituted acylamino, optionally substituted aminoacyloxy, optionally substituted oxyacylamino, optionally substituted oxyacyloxy or optionally substituted thio or optionally substituted phosphoryl;
R₂ is independently selected from halogen, cyano, optionally substituted alkyl, optionally substituted alkenyl or optionally substituted alkynyl;
u is an integer selected from 3 to 15;
each s is an integer independently selected from 0 to 4; and
q is an integer selected from 2 to 5;
q′ is an integer selected from 2 to 5; and
b) flowing the cell and/or a lipid vesicle through the flow cytometer.

In some embodiments, R₂ is independently selected from halogen, cyano, optionally substituted alkyl, optionally substituted alkenyl or optionally substituted alkynyl. In other embodiments, R₂ is independently selected from halogen, cyano or optionally substituted alkyl. In other embodiments, R₂ is independently selected from halogen, cyano, methyl, ethyl or propyl.

In some embodiments, u is an integer selected from 3 to 14. In other embodiments, u is an integer selected from 3 to 12, 3 to 10, 3 to 9, 3 to 8, 3 to 7, 4 to 7, or 4 to 6. In other embodiments, u is 3, 4, 5, 6 or 7. In some embodiments, u is 4, 5 or 6. In other embodiments, u is 4.

Examples of salt or solvate forms of compound of Formula (IV) are given below. The examples of compound of Formula (IV) are also within the scope of the present disclosure.

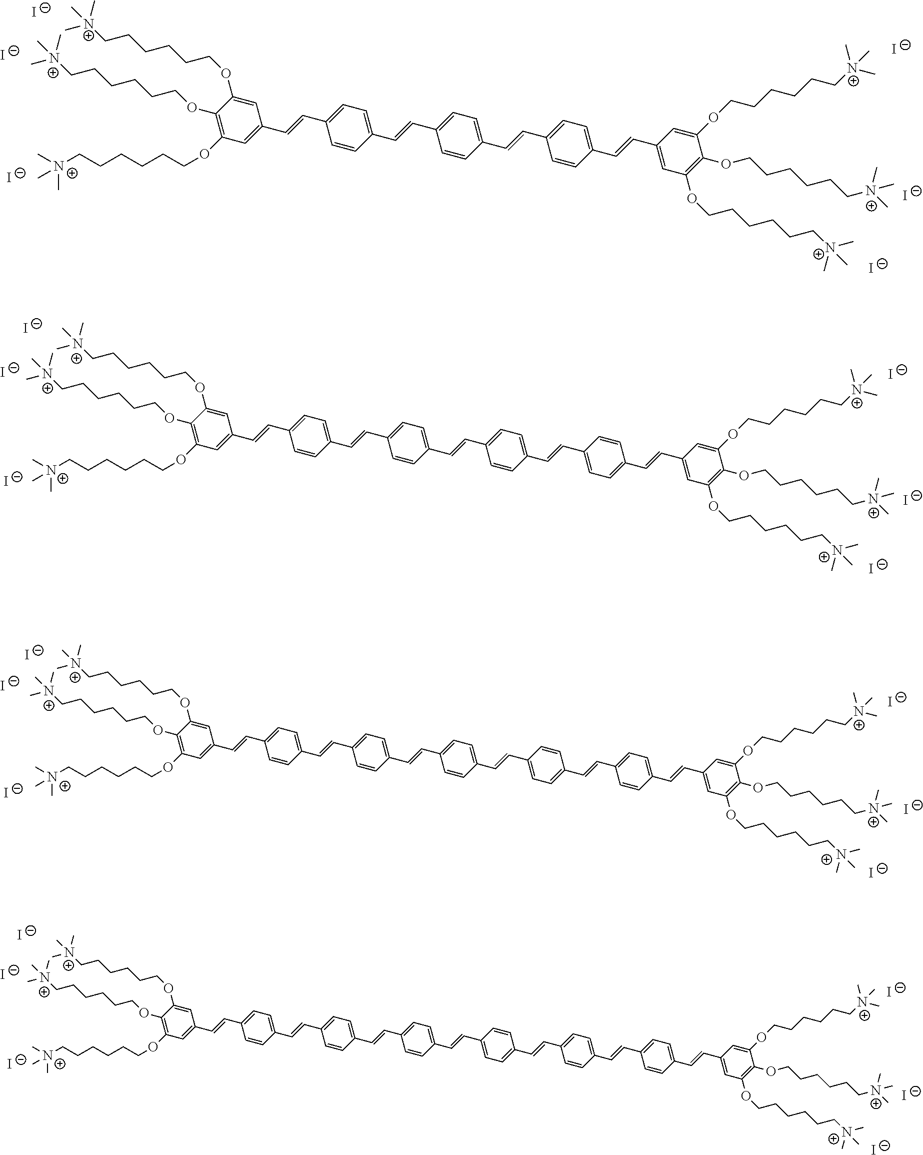

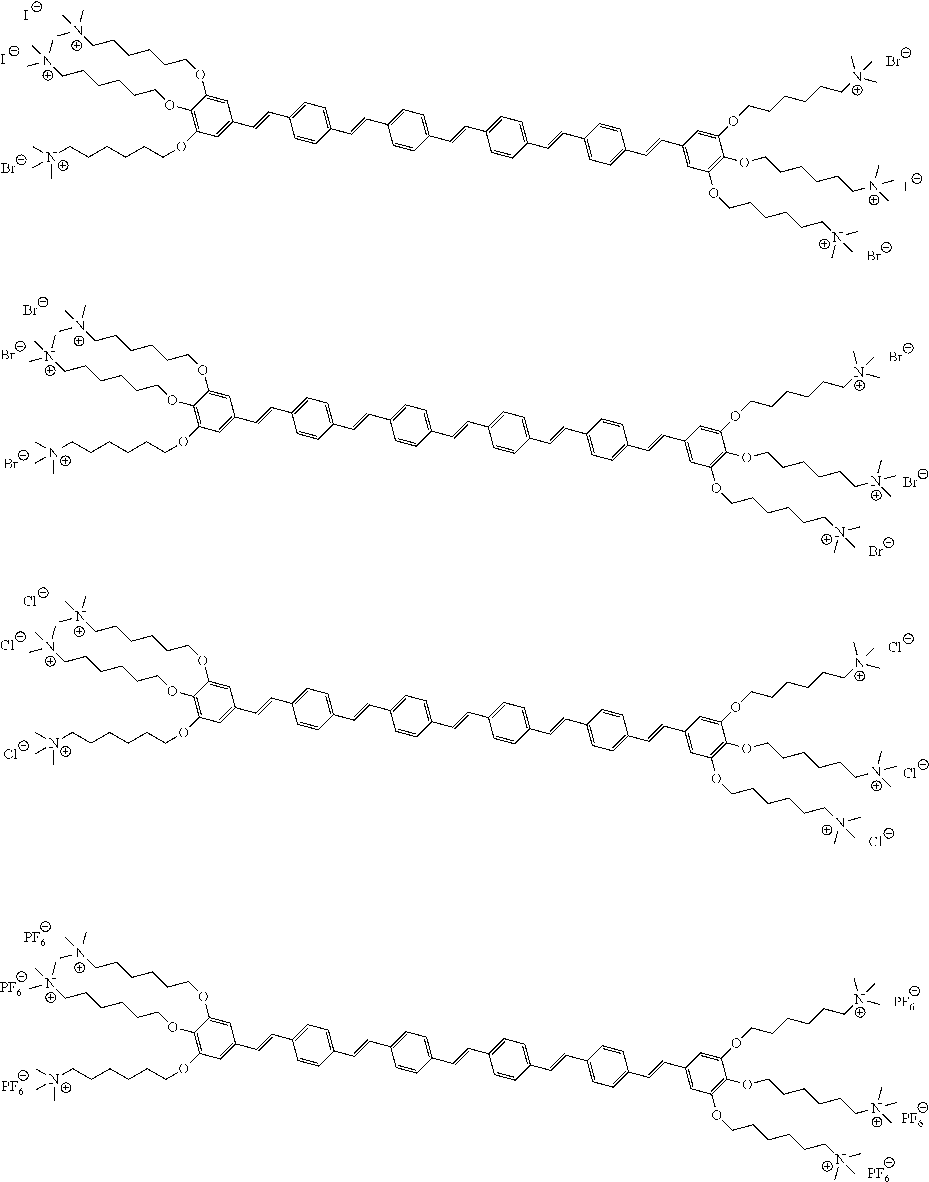

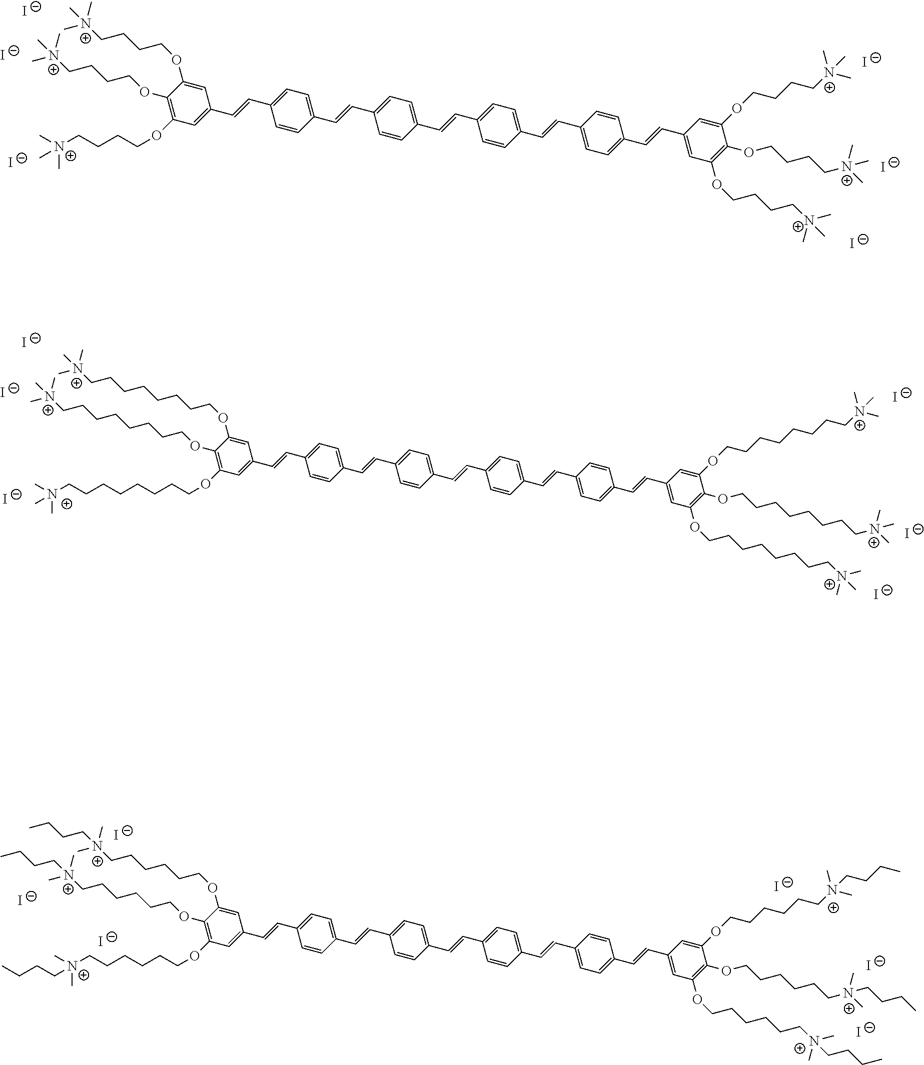

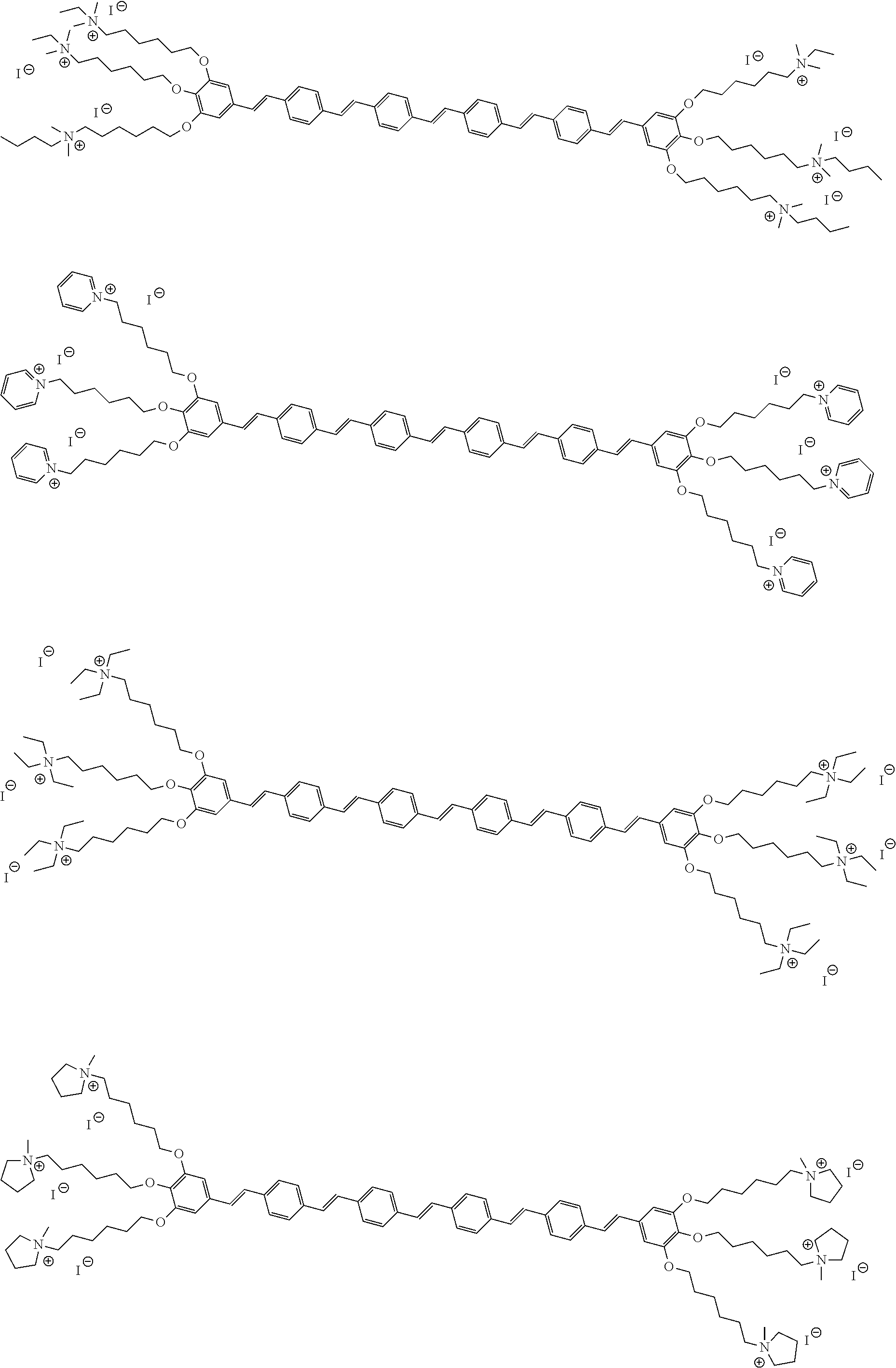

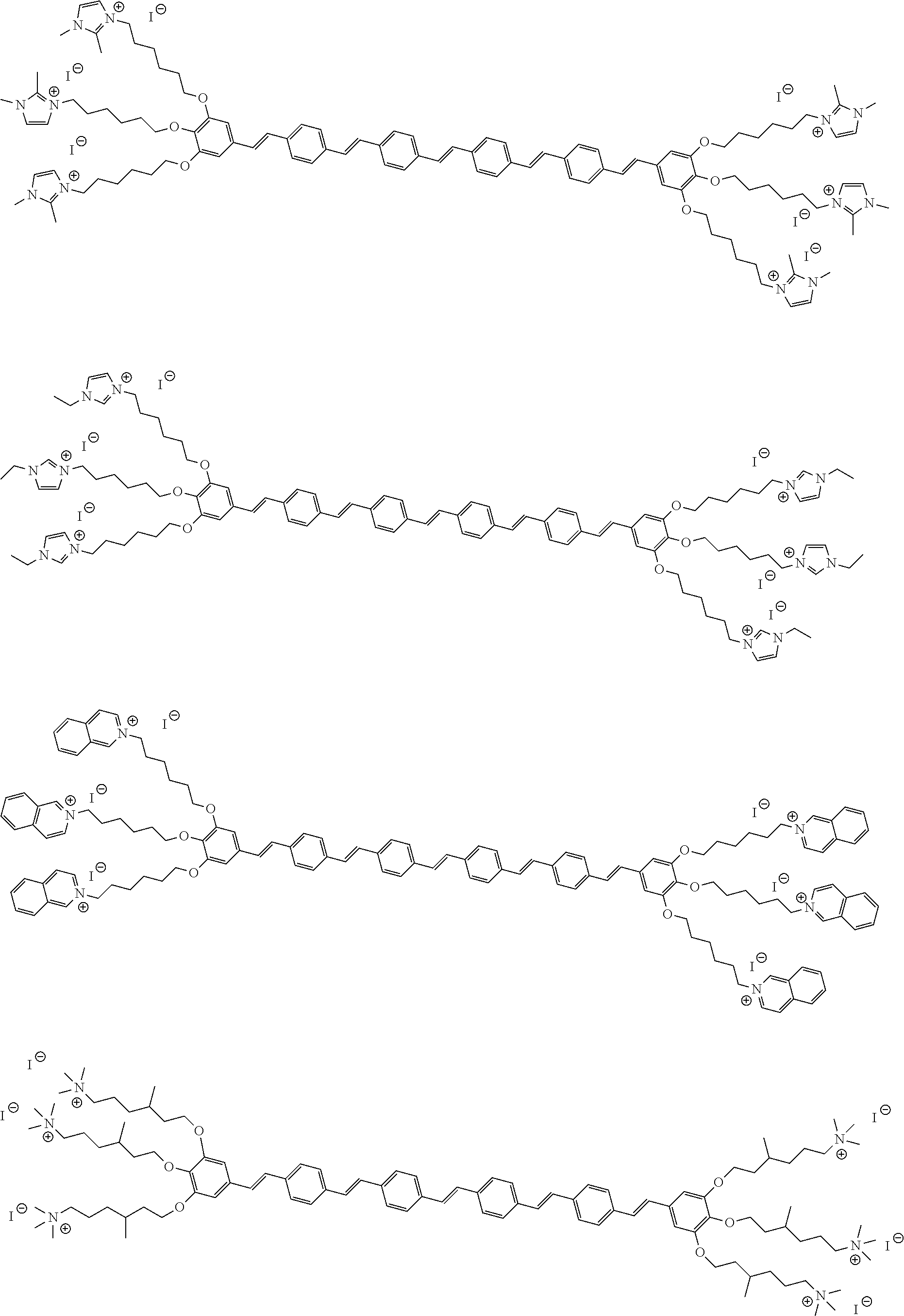

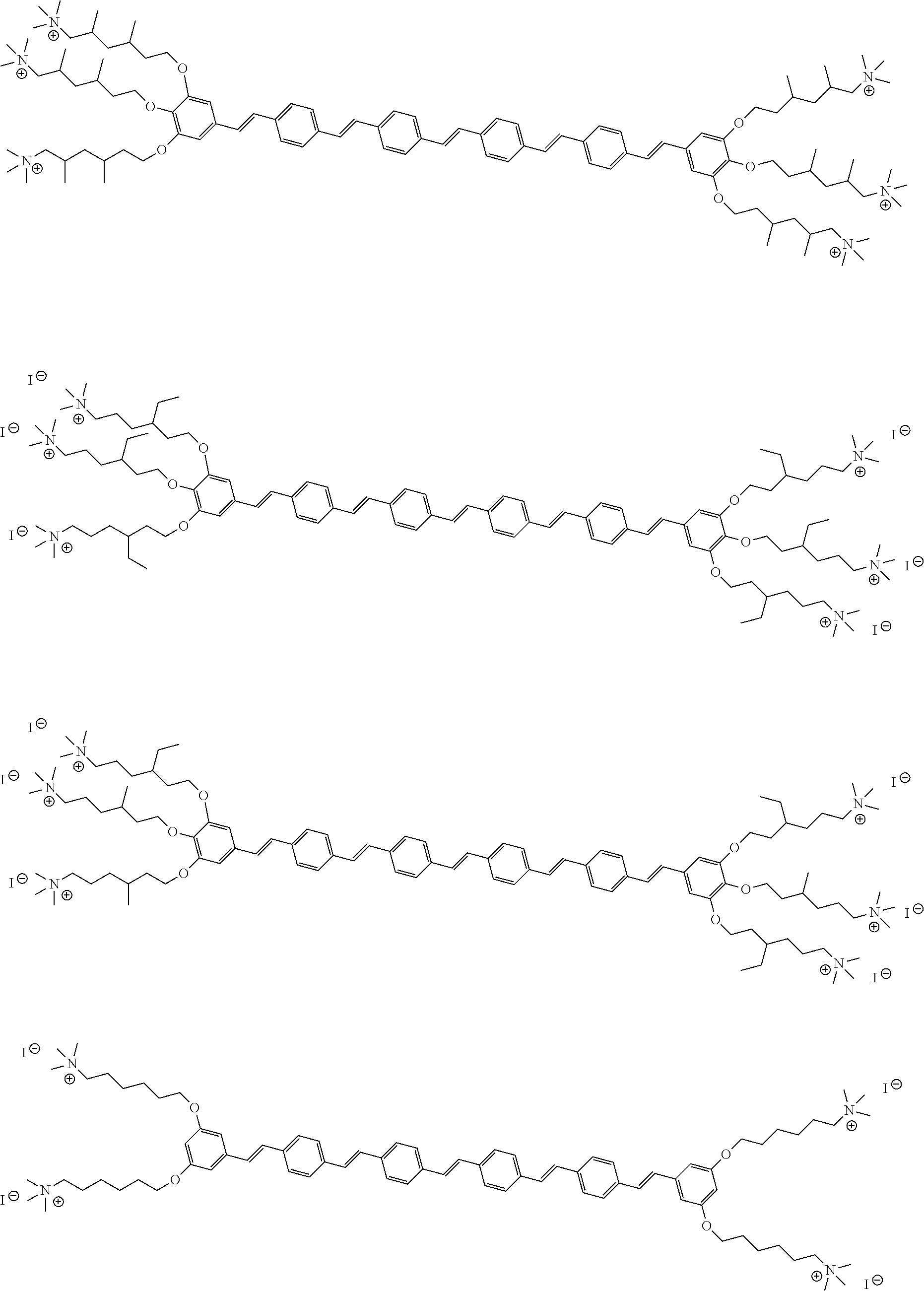

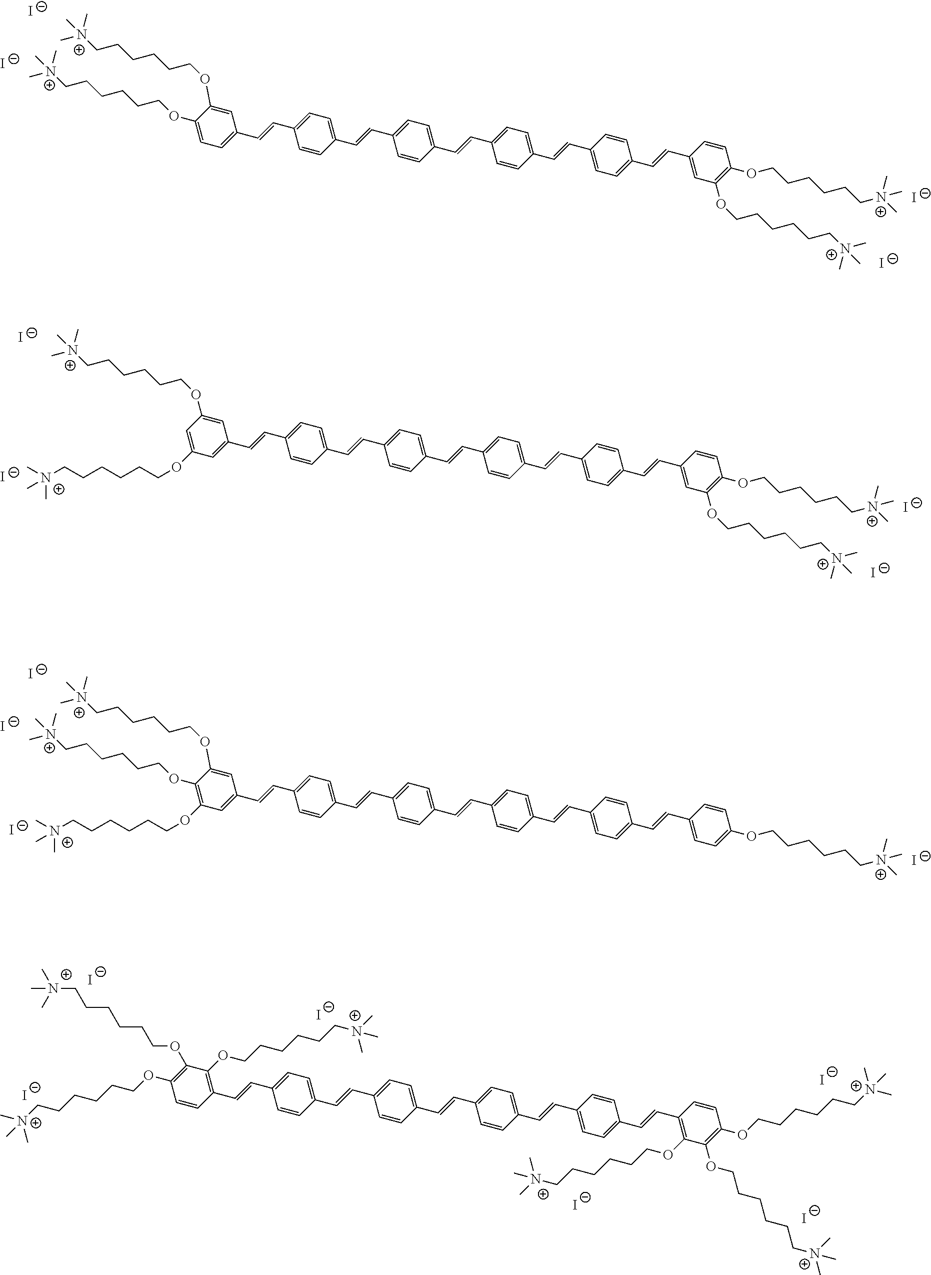

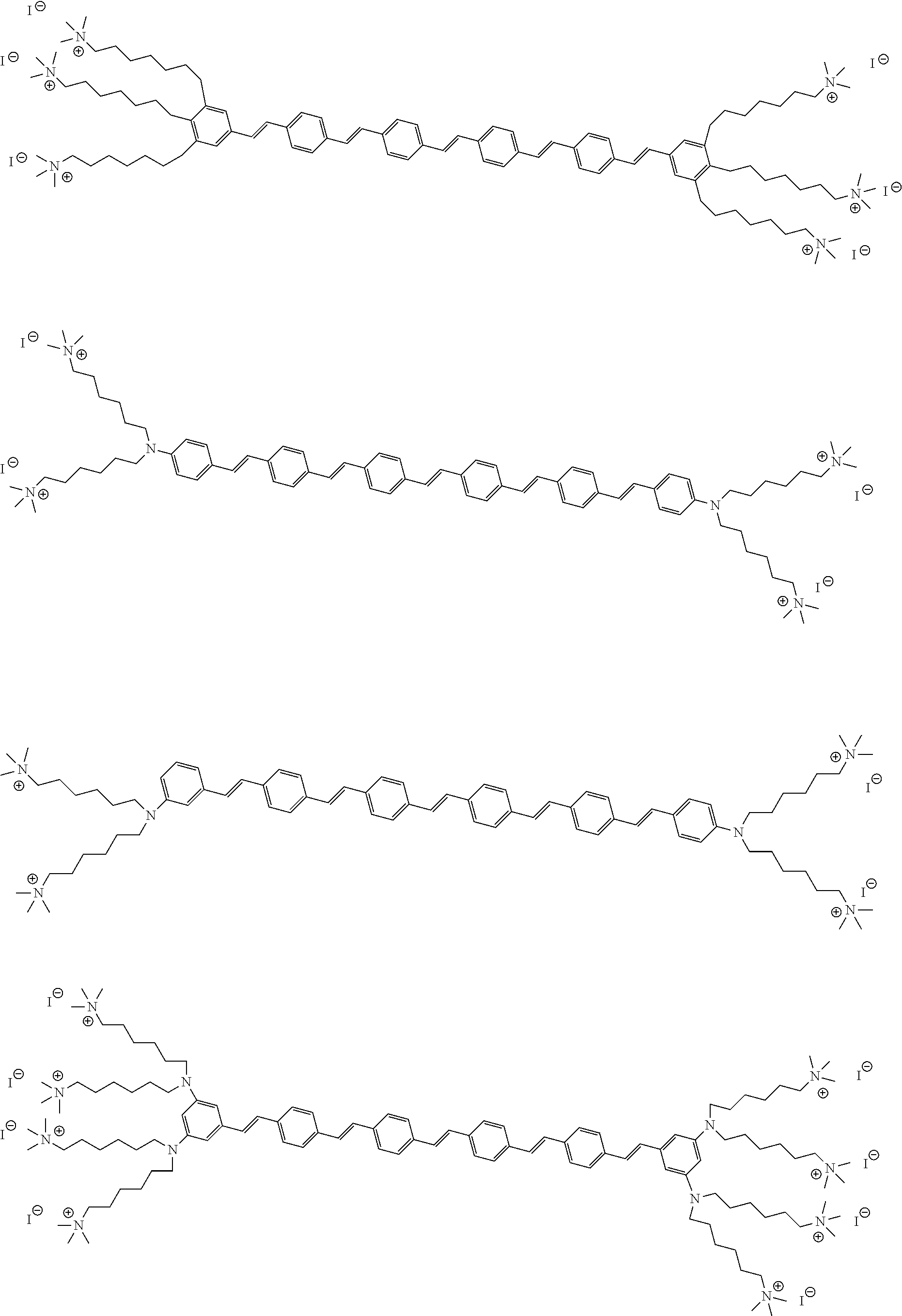

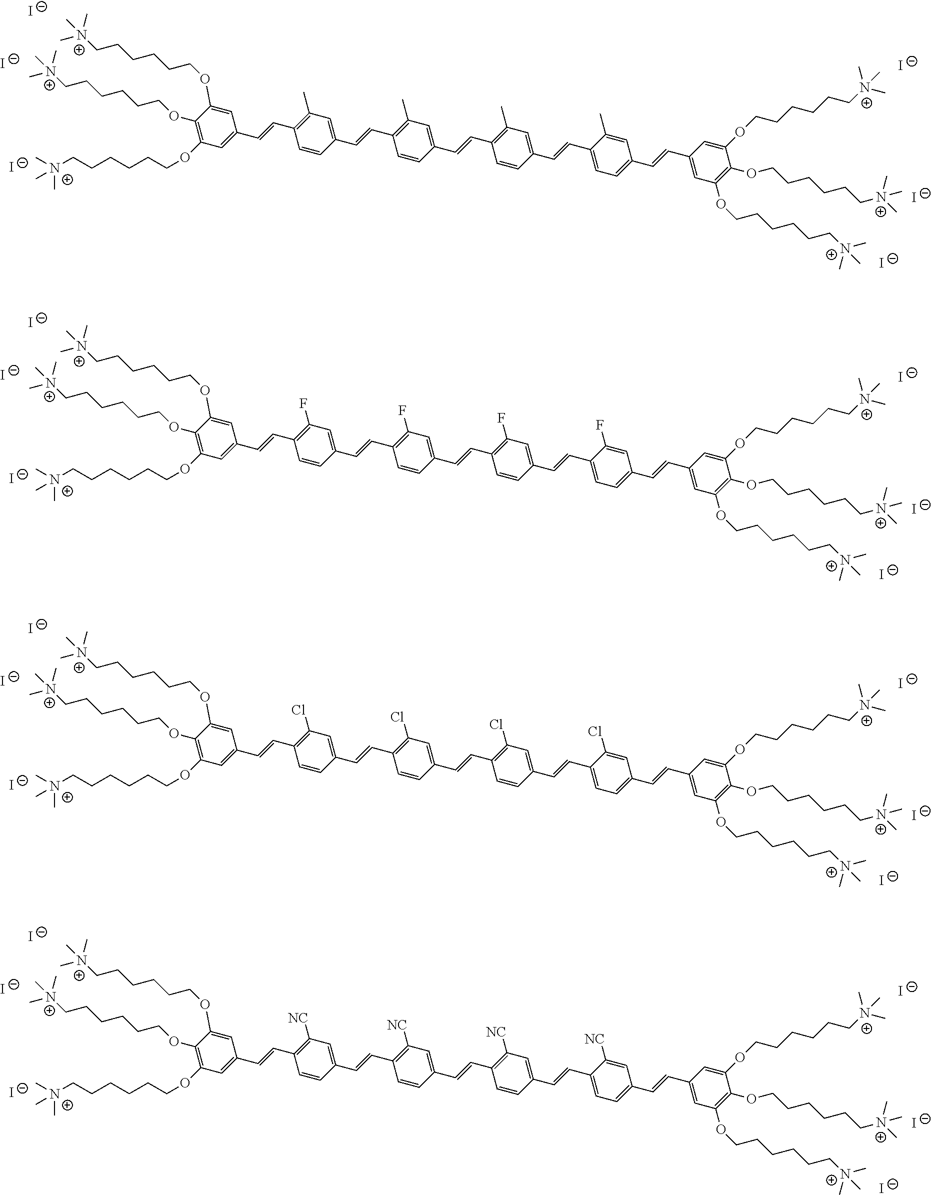

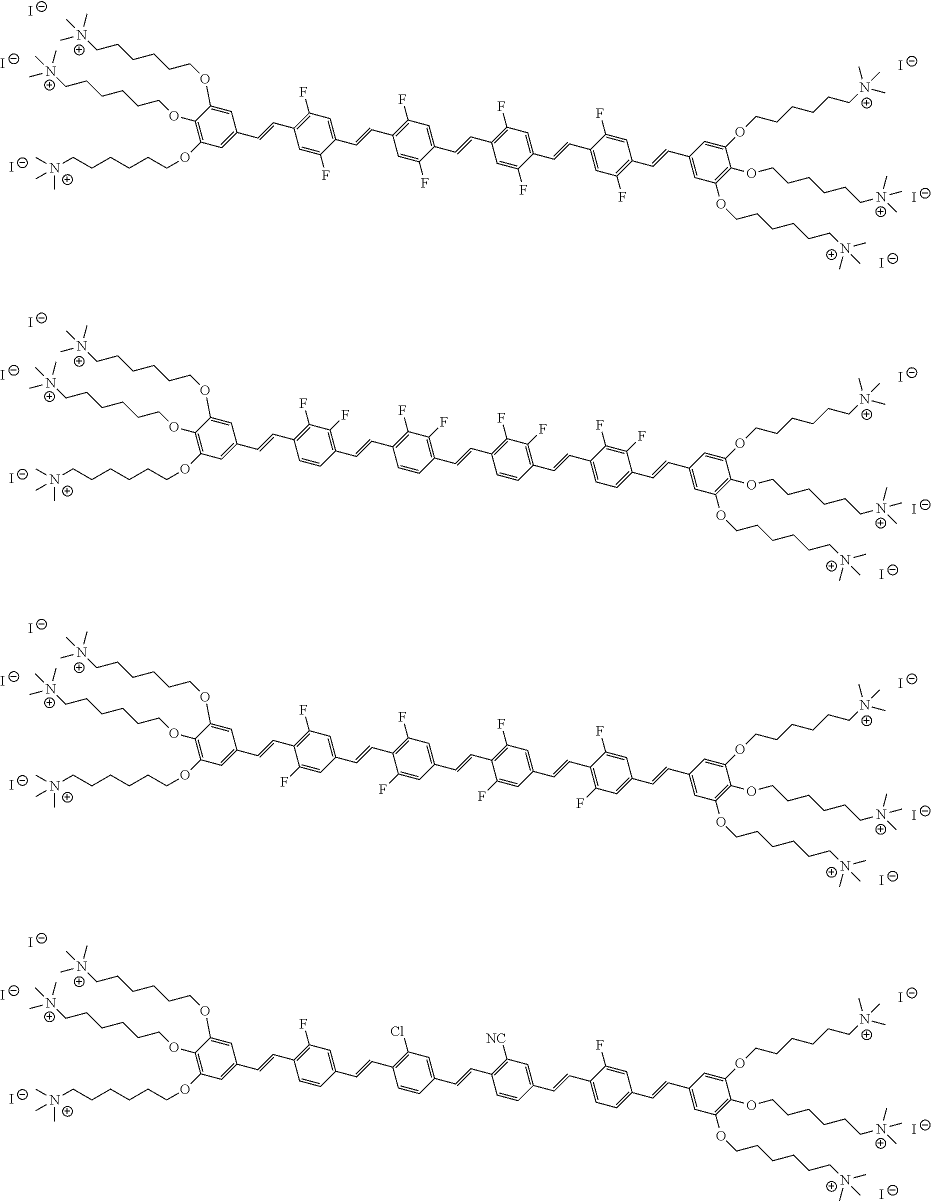

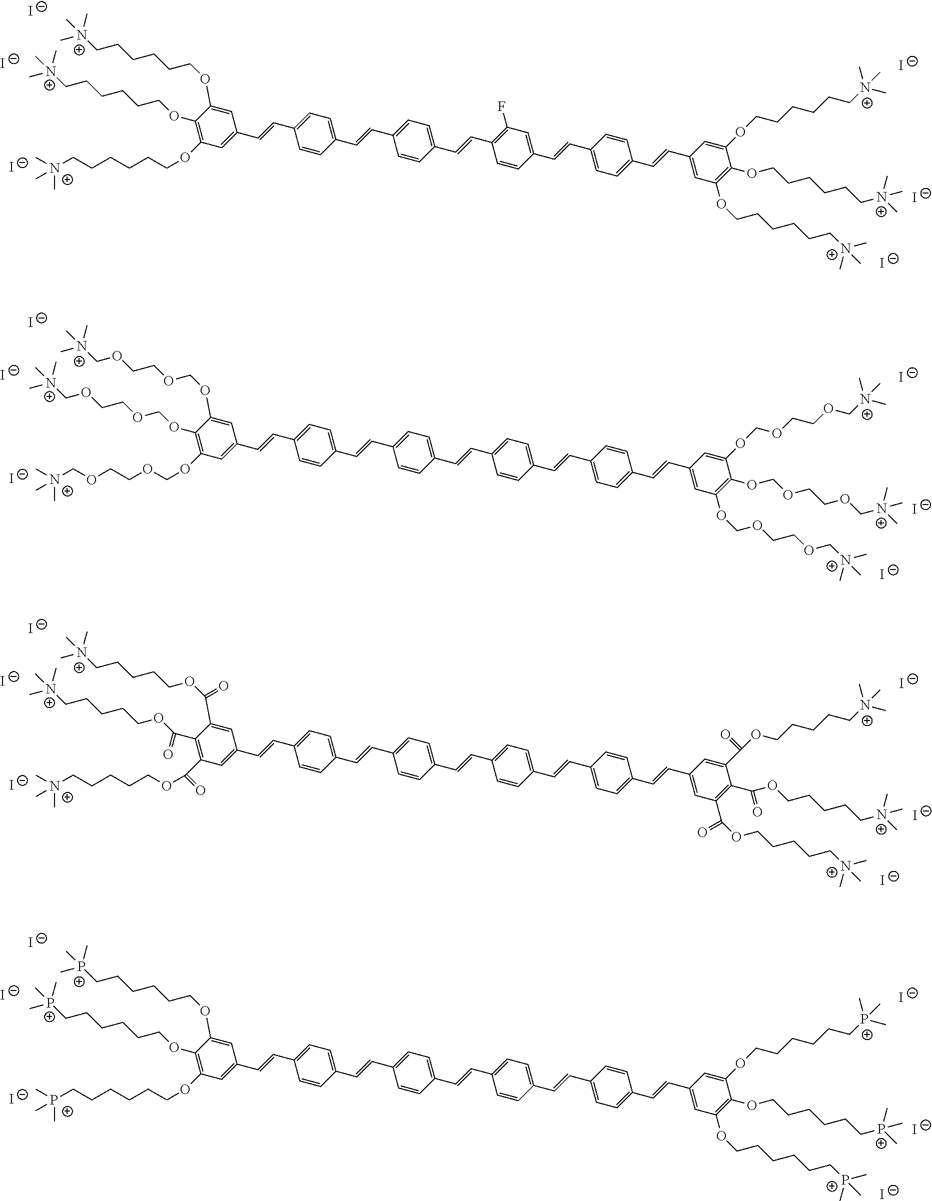

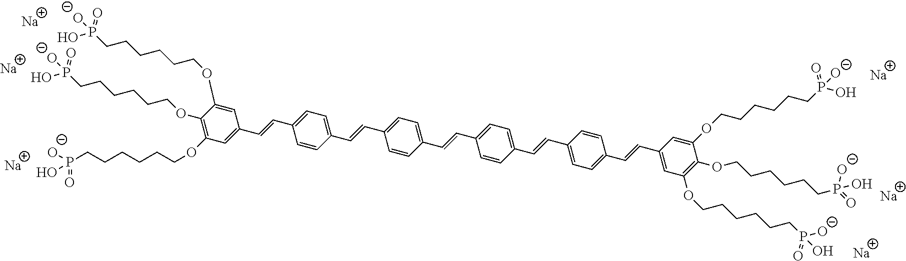

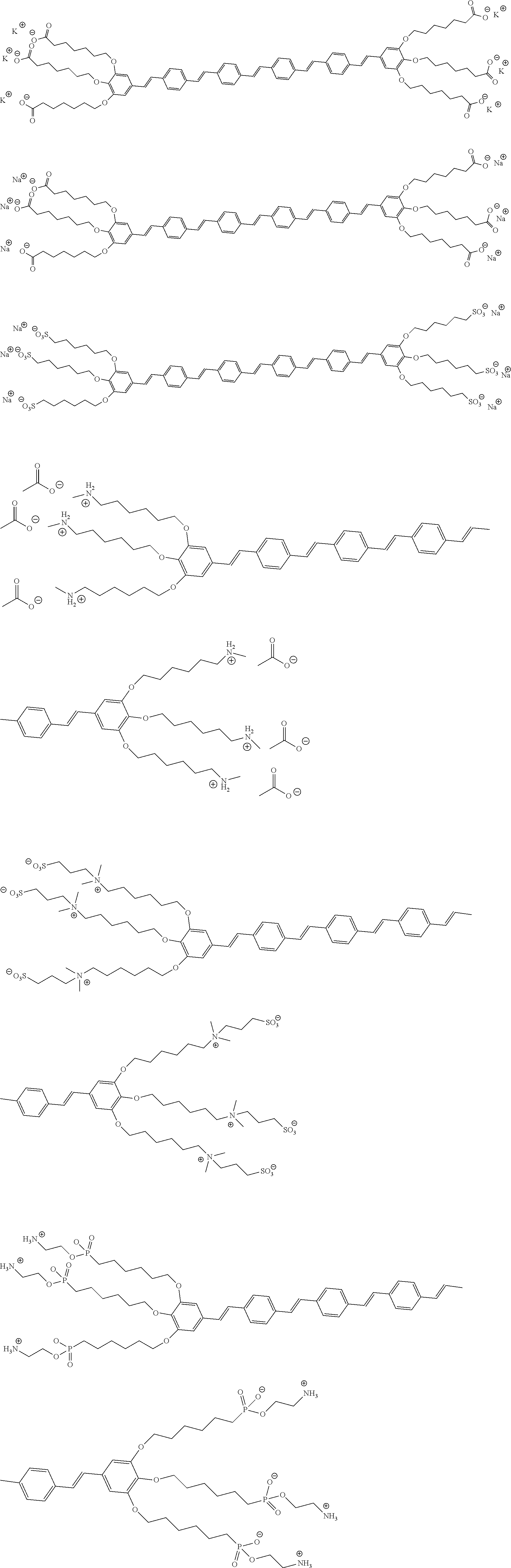

The cells can be adherent cells, or can be in suspension. The lipid vesicles can be exosomes (membrane-bound extracellular vesicles that are produced in the endosomal compartment of eukaryotic cells), synthetic and non-synthetic liposomes, or lipid nanoparticles (spherical vesicles made of ionizable lipids, which are positively charged at low pH and neutral at physiological pH). Advantageously, it was found that after intercalation of cell membranes by the fluorescence probe, the fluorescence probe can perpetuate to later cell populations.

The cell and/or a lipid vesicle is stained by the compound of Formula (I), sub-Formula (Ia-Ig), and/or Formula (IV) or a salt or solvate thereof after the incubation step. In some embodiments, the incubation period is about 1 min to about 12 days, about 1 min to about 10 days, about 1 min to about 8 days, about 1 min to about 6 days, about 1 min to about 5 days, about 1 min to about 4 days, about 1 min to about 3 days, about 1 min to about 2 days, about 1 min to about 24 h, about 1 min to about 20 h, about 1 min to about 16 h, about 1 min to about 12 h, about 1 min to about 10 h, about 1 min to about 9 h, about 1 min to about 8 h, about 1 min to about 7 h, about 1 min to about 6 h, about 5 min to about 6 h, about 5 min to about 5.5 h, about 5 min to about 5 h, about 5 min to about 4.5 h, about 5 min to about 4 h, about 5 min to about 3.5 h, about 5 min to about 3 h, about 5 min to about 2.5 h, about 5 min to about 2 h, about 5 min to about 1.5 h, about 5 min to about 1 h, about 5 min to about 50 min, about 5 min to about 40 min, about 5 min to about 30 min, about 5 min to about 20 min, or about 5 min to about 10 min. In other embodiments, the incubation period is about 10 min.

By controlling the concentration of the compound of Formula (I), sub-Formula (Ia-Ig), and/or Formula (IV) or a salt or solvate thereof, a sufficient amount of time can be obtained for analysis of the sample. In some embodiments, cell and/or lipid vesicle is passed through the flow cytometer within 30 min from contacting the compound of Formula (I) and/or Formula (IV) to the sample. In other embodiments, the time is within 10 min, 20 min, 30 min, 40 min, 50 min, 1 h, 2 h, 3 h, 4 h, 5 h, 6 h, 8 h, 10 h, 12 h, 16 h, 20 h, 24 h, 2 days, 3 days, 4 days, 5 days, 6 days, 8 days, 10 days, or 12 days. In other embodiments, the time is more than about 30 min, about 1 h, about 2 h, about 3 h, about 4 h, about 5 h, about 6 h, about 8 h, about 10 h, about 12 h, about 16 h, about 20 h, about 24 h, about 2 days, about 3 days, about 4 days, about 5 days, about 6 days, about 8 days, about 10 days, or about 12 days.

In some embodiments, the cell and/or lipid vesicle can be flowed through a flow cytometer without a purification step. This is possible as the free compound of Formula (I), sub-Formula (Ia-Ig), and/or Formula (IV) or a salt or solvate thereof are weakly emissive and will yield less background.

By flowing the cell and/or lipid vesicle through the flow cytometer, the cell and/or lipid vesicle can be excited by an electromagnetic radiation and subsequently detected by a detector. Accordingly, the method may further include a step of exposing the cell and/or lipid vesicle to electromagnetic radiation having a wavelength of less than about 2500 nm, or less than about 1000 nm.

In some embodiments, compound of Formula (I), sub-Formula (Ia-Ig), and/or Formula (IV) or a salt or solvate thereof has a fluorescence excitation in the wavelength of about 300 nm to about 1000 nm, or about 400 nm to about 700 nm. Depending on the combination of D and A, the excitation can be tunable within this wavelength range.

In some embodiments, the compound of Formula (I), sub-Formula (Ia-Ig), and/or Formula (IV) or a salt or solvate thereof has a fluorescence excitation peak with a full width half maximum (FWHM) of about 10 nm to about 200 nm. In other embodiments, the FWHM is about 10 nm to about 190 nm, about 10 nm to about 180 nm, about 10 nm to about 170 nm, about 10 nm to about 160 nm, about 10 nm to about 150 nm, about 10 nm to about 140 nm, about 10 nm to about 130 nm, about 10 nm to about 120 nm, about 10 nm to about 110 nm, or about 10 nm to about 100 nm. In other embodiments, the FWHM is about 20 nm to about 200 nm, about 30 nm to about 200 nm, about 40 nm to about 200 nm, about 50 nm to about 200 nm, about 60 nm to about 200 nm, about 70 nm to about 200 nm, about 80 nm to about 200 nm, about 90 nm to about 200 nm, or about 100 nm to about 200 nm.

In some embodiments, compound of Formula (I), sub-Formula (Ia-Ig), and/or Formula (IV) or a salt or solvate thereof has a fluorescence emission in the wavelength of about 300 nm to about 2000 nm. In other embodiments, the range is about 300 nm to about 1900 nm, about 300 nm to about 1800 nm, about 300 nm to about 1700 nm, about 300 nm to about 1600 nm, or about 300 nm to about 1500 nm. Depending on the combination of D and A, the excitation can be tunable within this wavelength range.

In some embodiments, the compound of Formula (I), sub-Formula (Ia-Ig), and/or Formula (IV) or a salt or solvate thereof has a fluorescence emission peak with a full width half maximum (FWHM) of about 10 nm to about 200 nm. In other embodiments, the FWHM is about 10 nm to about 190 nm, about 10 nm to about 180 nm, about 10 nm to about 170 nm, about 10 nm to about 160 nm, about 10 nm to about 150 nm, about 10 nm to about 140 nm, about 10 nm to about 130 nm, about 10 nm to about 120 nm, about 10 nm to about 110 nm, or about 10 nm to about 100 nm. In other embodiments, the FWHM is about 20 nm to about 200 nm, about 30 nm to about 200 nm, about 40 nm to about 200 nm, about 50 nm to about 200 nm, about 60 nm to about 200 nm, about 70 nm to about 200 nm, about 80 nm to about 200 nm, about 90 nm to about 200 nm, or about 100 nm to about 200 nm.

In some embodiments, when the compound of Formula (I), sub-Formula (Ia-Ig), and/or Formula (IV) or a salt or solvate thereof is inserted into the cellular and/or lipid membrane, the compound of Formula (I), sub-Formula (Ia-Ig), and/or Formula (IV) or a salt or solvate thereof has an emission intensity of more than about 2 times to about 500 times relative to a control sample of the compound of Formula (I), sub-Formula (Ia-Ig), and/or Formula (IV) or a salt or solvate thereof. In other embodiments, the emission intensity is more than about 10 times to about 500 times, about 20 times to about 500 times, about 30 times to about 500 times, about 40 times to about 500 times, about 50 times to about 500 times, about 60 times to about 500 times, about 70 times to about 500 times, about 80 times to about 500 times, about 90 times to about 500 times, about 100 times to about 500 times, about 100 times to about 450 times, about 150 times to about 450 times, about 200 times to about 450 times, about 250 times to about 450 times, about 300 times to about 450 times, or about 350 times to about 450 times.

A control in an experiment is a group separated from the rest of the experiment, where the independent variable being tested cannot influence the results. When testing a sample of cells and/or lipid vesicles, the at least one control sample may comprise a compound of Formula (I), sub-Formula (Ia-Ig), and/or Formula (IV) or a salt or solvate thereof in an aqueous/water medium. In this regard, the control sample does not contain cells and/or lipid vesicles.

Advantageously, compounds of Formula (I), sub-Formula (Ia-Ig), and/or Formula (IV) or a salt or solvate thereof have a low (or negligible) photoluminescence when dissolved in an aqueous medium. However, when partitioned into the lipid bilayer, the photoluminescence of compound of Formula (I), sub-Formula (Ia-Ig), and/or Formula (IV) or a salt or solvate thereof is enhanced. It is believed that the change in local environment to a hydrophobic one (alkyl chains of the lipid bilayer) allows for the enhancement of fluorescence. Fluorescence is the emission of light by a substance that has absorbed light or other electromagnetic radiation. In general, the emitted light is of a longer wavelength than the absorbed light.

Because compound of Formula (I), sub-Formula (Ia-Ig), and/or Formula (IV) or a salt or solvate thereof are advantageously non-toxic (or have a low toxicity) and/or have stable to excitation, when under constant excitation, the photoluminescence intensity may be maintained for a period of time. In this regard, the photoluminescence intensity does not decrease for some time when the compounds are excited under the appropriate wavelength for imaging. This is believed to be due to the conjugation system, which allows for the dissipation and transfer of energy, thus preventing localised heating and degradation of the compound. In some embodiments, the photoluminescence intensity may be maintained for at least 20 min, at least 30 min, at least 40 min, at least 50 min, at least 60 min, at least 1.5 h, at least 2 h, at least 3 h, at least 4 h, at least 6 h, at least 10 h or at least 24 h.

Compounds of Formula (I), sub-Formula (Ia-Ig), and/or Formula (IV) or a salt or solvate thereof can work in combination with other dyes, for example membrane dye. For example, a commercial available dye, FM4-64, can be added to recognize bacterial envelope type in-situ in the bacteria mixture. In this regard, a dual-dye system that can recognise polymicrobial samples is also disclosed. These methods are easy-to-use requiring only a simple application of a dye mixture with no fixation or other pre-treatment requirement, and compound of Formula (I), sub-Formula (Ia-Ig), and/or Formula (IV) or a salt or solvate thereof is stable in aqueous solution and can be used to monitor the cells in a living system.

Accordingly, in an embodiment, the method further comprises a step of contacting the cell and/or lipid vesicle with another dye. The dye can be used to stain cell membranes, nucleus, DNA, RNA, or other organelles in the cell. The dye can be a fluorescence probe, such as FM4-64, FM 2-10, FM 1-43, Propidium Iodide, SYTO 82, SYTO 83, SYTO 84, SYTO 85, YOYO®-3 iodide, YO-PRO™-3 Iodide, BOBO™-3 Iodide, Ethidium Homodimer-1, Ethidium Homodimer-2, Ethidium monoazide, Acridine Orange, CellMask™ Plasma Membrane Stains or Di-4-ANEPPS.

The present invention also provides a flow system for detecting and/or quantifying cells and/or lipid vesicles, comprising:
a) a compound of Formula (I), sub-Formula (Ia-Ig), and/or Formula (IV) or a salt or solvate thereof for labelling the cells and/or the lipid vesicles;
b) an inlet for introducing the labelled cells and/or lipid vesicles into the flow system;
c) a detection means in fluid communication with the inlet for detecting a fluorescence emission from the labelled cells and/or lipid vesicles; and
d) optionally a counter means for quantifying the labelled cells and/or lipid vesicles.

The flow system can for example be a microfluidic chip.

In some embodiments, the flow system further comprises an incubation means. The incubation means allows the compound of Formula (I), sub-Formula (Ia-Ig), and/or Formula (IV) or a salt or solvate thereof to intercalate with the cell membrane or within the lipid bilayer.

In some embodiments, the detection means is a fluorescence detector.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### General Protocol for the Synthesis of Compound of Formula (I)

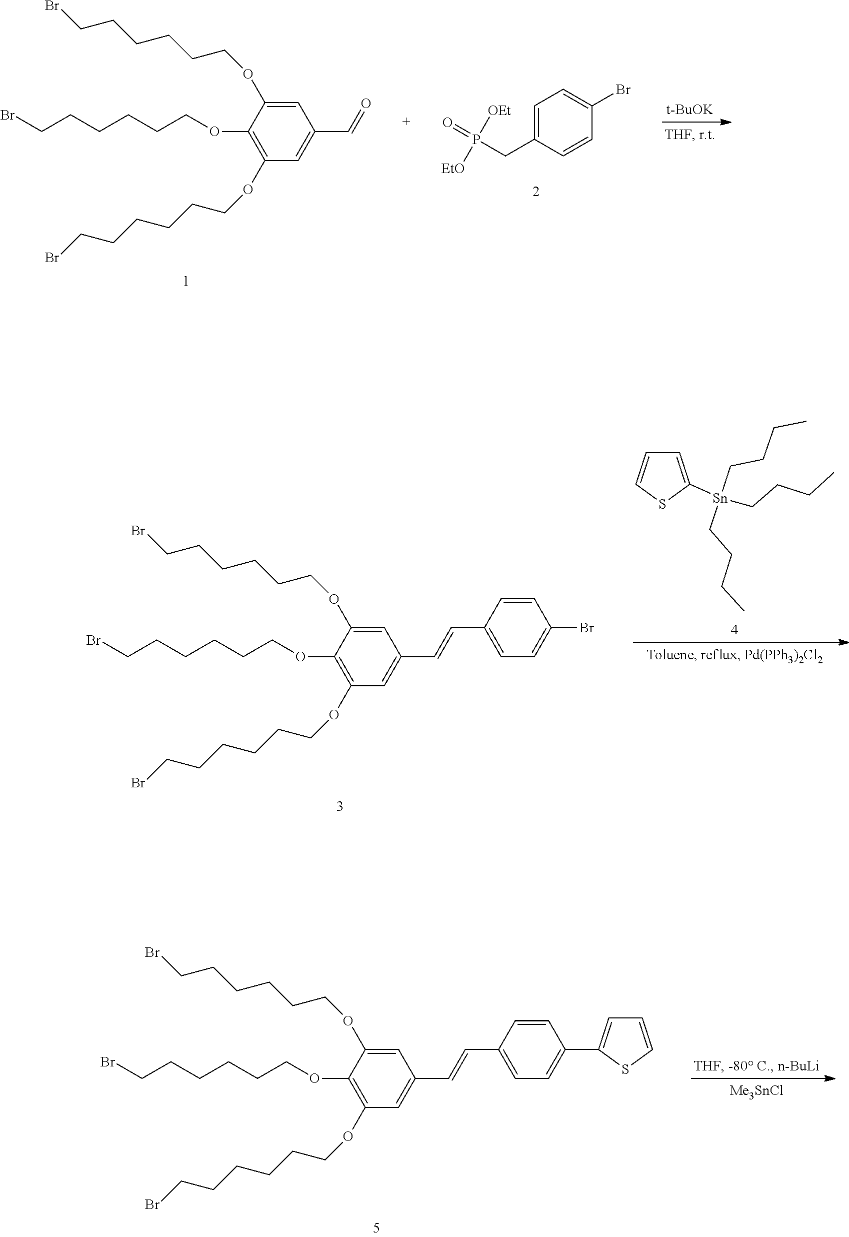

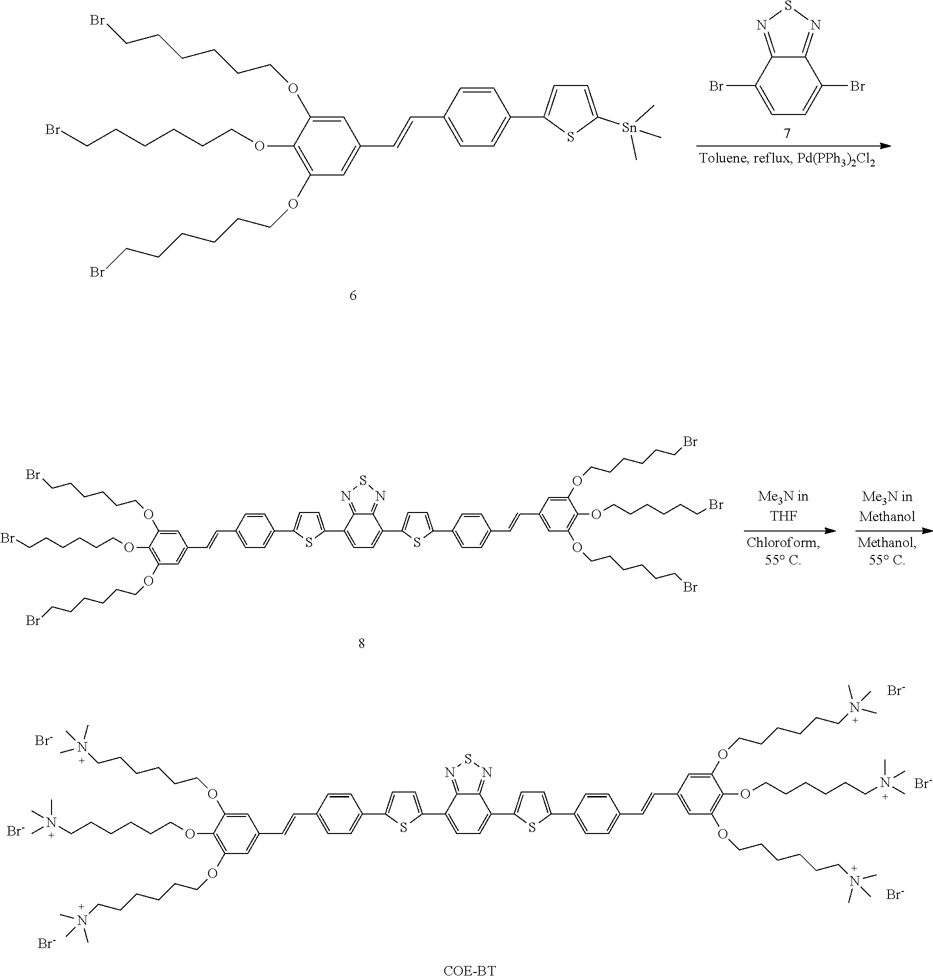

### Synthetic Route of Compound COE-BT.

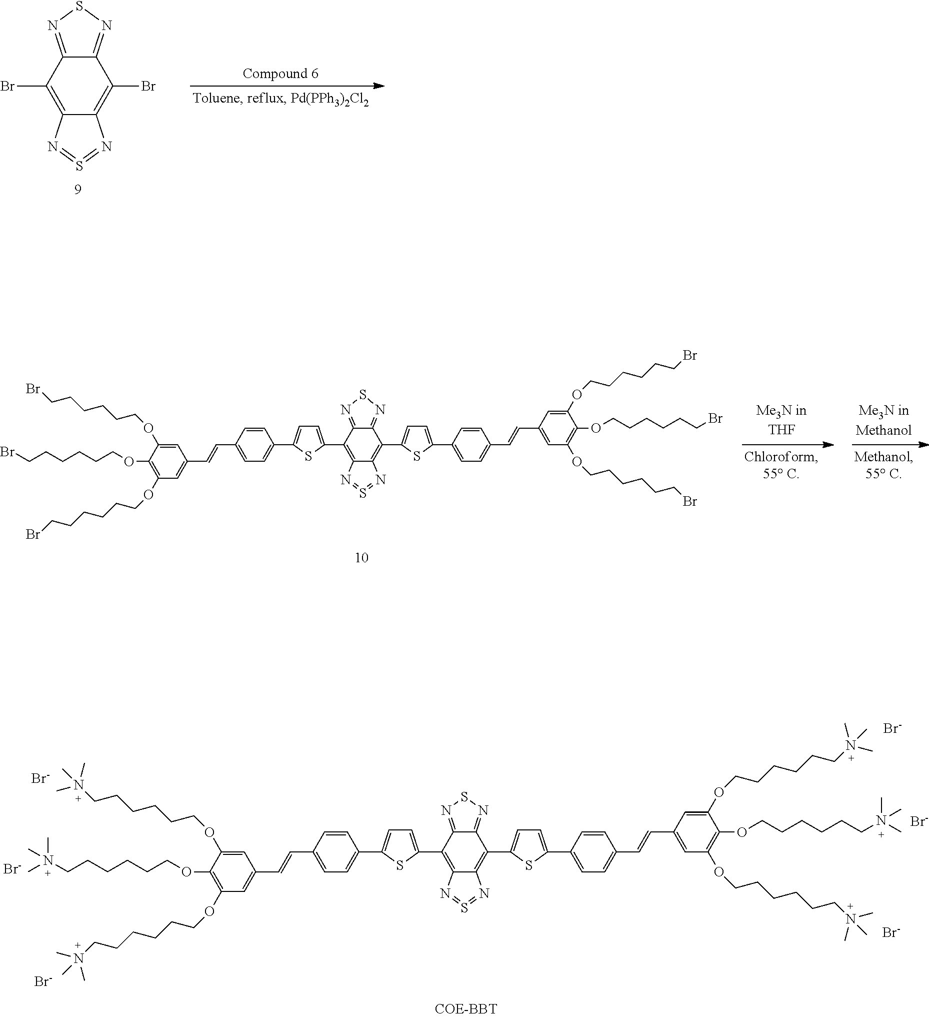

### Synthetic Route of Compound COE-BBT.PP883

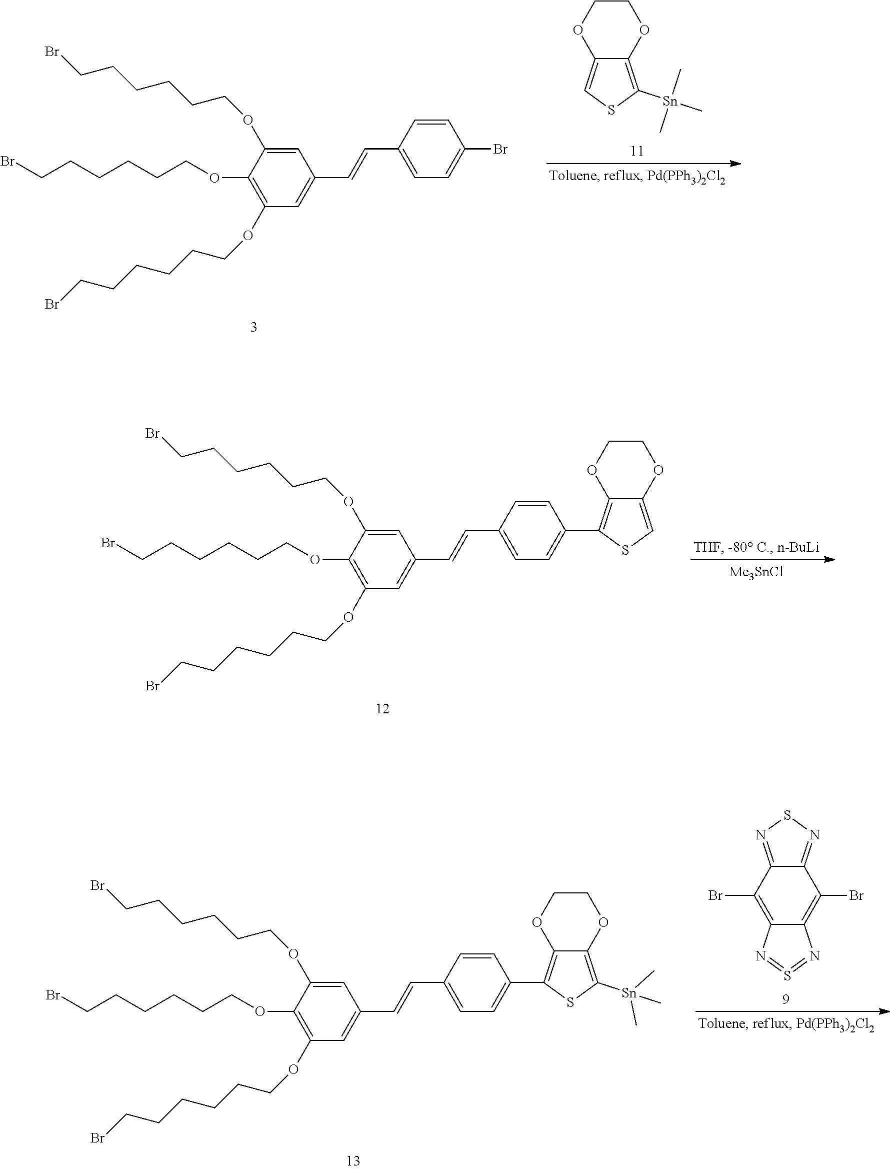

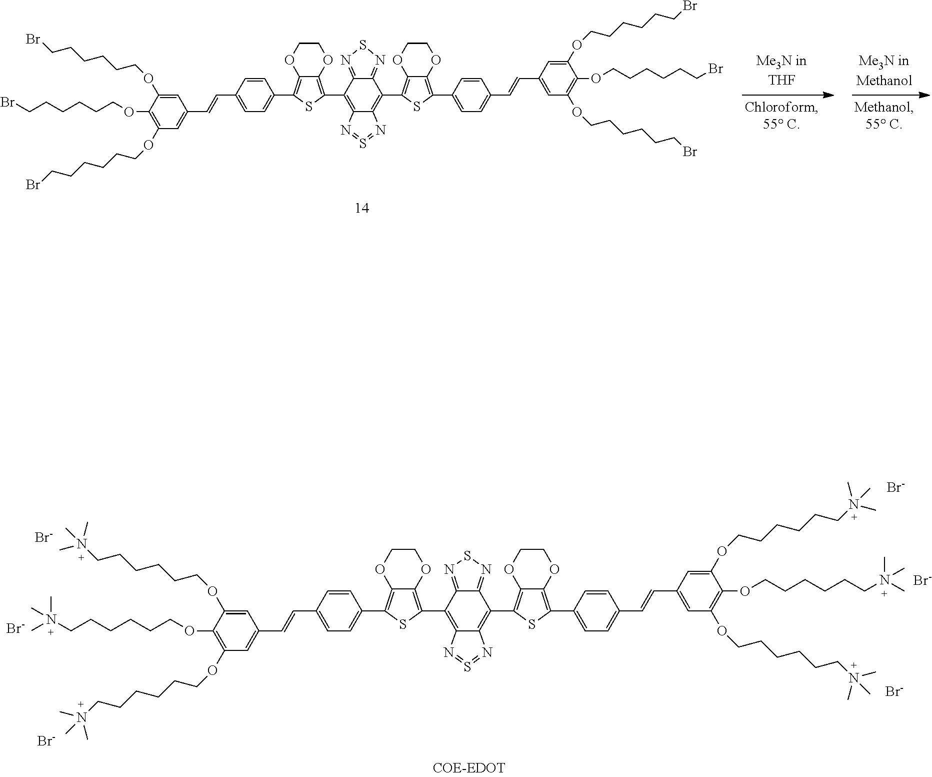

### Synthetic Route of Compound COE-EDOT.

### (E)-1,2,3-tris((6-bromohexyl)oxy)-5-(4-bromostyryl)benzene (compound 3)

Compound 1 (5.69 g, 8.46 mmol), compound 2 (4.07 g, 13.27 mmol), potassium tert-butoxide (0.99 g, 8.46 mmol), and 100 mL dry THF were added to a round flask under the protection of nitrogen atmosphere. After 16 h reaction under stirring at room temperature, the reaction mixture was poured into water, and extracted with chloroform. The transparent organic phase was dried over Na₂SO₄ and then removed from the organic solvent by a rotary evaporator. The crude product was purified with column chromatography using hexane and dichloromethane as eluent, and then the product was obtained as white solid (6.63 g, 94% yield). ¹H NMR (400 MHz, Chloroform-d) δ 7.49-7.44 (m, 2H), 7.37-7.33 (m, 2H), 6.99 (d, J=16.2 Hz, 1H), 6.90 (d, J=16.2 Hz, 1H), 6.70 (s, 2H), 4.06-4.00 (m, 4H), 3.99-3.95 (m, 2H), 3.49-3.39 (m, 6H), 1.96-1.71 (m, 12H), 1.60-1.47 (m, 12H). ¹³C NMR (101 MHz, CDCl₃) δ 153.56, 138.68, 136.62, 132.69, 132.12, 129.82, 128.18, 126.95, 121.51, 105.65, 73.58, 69.27, 34.25, 34.12, 33.20, 33.07, 30.48, 29.61, 28.44, 28.27, 25.70, 25.68.

### (E)-2-(4-(3,4,5-tris((6-bromohexyl)oxy)styryl)phenyl)thiophene (compound 5)

Compound 3 (4.44 g, 5.58 mmol), compound 4 (4.16 g, 11.15 mmol), Pd(PPh₃)₂Cl₂ (157 mg, 0.223 mmol) were added to a round flask under the protection of nitrogen atmosphere. After purging using nitrogen, 50 mL dry toluene was added to the reaction mixture, which was then heated at 120° C. for 16 hours with stirring. After being cooled to room temperature, the reaction mixture was poured into water, and extracted with chloroform. The organic phase was dried over Na₂SO₄ and then the organic solvent was removed by rotary evaporator. The crude product was purified with silica gel column chromatography using hexane and dichloromethane as eluent, and the product was obtained as light yellow solid (4.02 g, 90% yield). ¹H NMR (400 MHz, Chloroform-d) δ 7.63-7.58 (m, 2H), 7.52-7.48 (m, 2H), 7.33 (dd, J=3.6, 1.2 Hz, 1H), 7.28 (dd, J=5.1, 1.1 Hz, 1H), 7.09 (dd, J=5.1, 3.6 Hz, 1H), 7.03 (d, J=16.2 Hz, 1H), 6.97 (d, J=16.3 Hz, 1H), 6.72 (s, 2H), 4.09-3.95 (m, 6H), 3.49-3.39 (m, 6H), 1.97-1.73 (m, 12H), 1.62-1.48 (m, 12H). ¹³C NMR (101 MHz, CDCl₃) δ 153.55, 144.48, 138.57, 136.85, 133.85, 133.03, 129.11, 128.44, 127.64, 127.20, 126.45, 125.15, 123.34, 105.65, 73.59, 69.29, 34.24, 34.11, 33.21, 33.09, 30.50, 29.64, 28.46, 28.28, 25.72, 25.70.

### (E)-trimethyl(5-(4-(3,4,5-tris((6-bromohexyl)oxy)styryl)phenyl)thiophen-2-yl)stannane (compound 6)

Compound 5 (2.88 g, 3.60 mmol) and 40 mL dry THF were added to a flask with nitrogen protection. The mixture was cooled down to −80° C. utilizing an ultralow temperature reaction bath. Then, 3.6 mL n-butyllithium solution in cyclohexane (2 M, 7.2 mmol) was added drop-wise into the reaction mixture. After stirring at −80° C. for 2 hours, 14.4 mL trimethyltin chloride solution in hexane (1 M, 14.4 mmol) was added and then the reaction mixture was warmed up to room temperature. After being stirred at room temperature overnight (about 16 hours), the reaction mixture was poured into water and extracted with hexane. The organic phase was washed using water four times and then dried over Na₂SO₄. After the solvent is removed using a rotary evaporator and vacuum pump, the crude product was obtained as a colorless oil, which will be used in subsequent reactions without further purification (3.34, 96% yield). ¹H NMR (400 MHz, Chloroform-d) δ 7.63-7.58 (m, 2H), 7.52-7.47 (m, 2H), 7.44 (d, J=3.3 Hz, 1H), 7.17 (d, J=3.3 Hz, 1H), 7.05-6.93 (m, 2H), 6.72 (s, 2H), 4.09-3.92 (m, 6H), 3.51-3.36 (m, 6H), 1.97-1.71 (m, 12H), 1.62-1.47 (m, 12H), 0.40 (s, 9H). ¹³C NMR (101 MHz, CDCl₃) δ 153.54, 150.19, 138.51, 138.18, 136.61, 136.59, 133.97, 133.09, 128.92, 127.74, 127.17, 126.47, 124.59, 105.60, 73.59, 69.27, 34.25, 34.12, 33.22, 33.09, 30.50, 29.64, 28.46, 28.29, 25.72, 25.70, -7.87.

### 4,7-bis(5-(4-((E)-3,4,5-tris((6-bromohexyl)oxy)styryl)phenyl)thiophen-2-yl)benzo[c][1,2,5]thiadiazole (compound 8)

Compound 6 (2.76 g, 2.87 mmol), compound 7 (281 mg, 0.88 mmol), Pd(PPh₃)₂Cl₂ (27 mg, 0.038 mmol) were added to a round flask under the protection of nitrogen atmosphere. After purging using nitrogen, 10 mL dry toluene was added to the reaction mixture, which was then heated at 120° C. for 16 hours with stirring. After being cooled to room temperature, the reaction mixture was poured into water, and extracted with chloroform. The organic phase was dried over Na₂SO₄ and then the organic solvent was removed by a rotary evaporator. The crude product was purified with silica gel column chromatography using hexane and dichloromethane as eluent. The product was dissolved in chloroform and precipitated using methanol, and the precipitation was collected by filtration and washed using methanol. After being dried in a vacuum, the product was obtained as a dark red solid (1.29 g, 78% yield). ¹H NMR (500 MHz, Chloroform-d) δ 8.12 (d, J=3.9 Hz, 2H), 7.89 (s, 2H), 7.69 (d, J=8.4 Hz, 4H), 7.53 (d, J=8.5 Hz, 4H), 7.43 (d, J=3.8 Hz, 2H), 7.05 (d, J=16.2 Hz, 2H), 6.99 (d, J=16.2 Hz, 2H), 6.73 (s, 4H), 4.08-4.02 (m, 8H), 4.01-3.96 (m, 4H), 3.47-3.40 (m, 12H), 1.96-1.74 (m, 24H), 1.60-1.47 (m, 24H). ¹³C NMR (126 MHz, CDCl₃) δ 153.55, 152.90, 145.65, 138.96, 138.57, 137.22, 133.48, 132.96, 129.31, 129.03, 127.54, 127.26, 126.30, 126.06, 125.64, 124.36, 105.60, 73.60, 69.26, 34.28, 34.15, 33.21, 33.09, 30.50, 29.64, 28.46, 28.29, 25.72, 25.71.

### Compound COE-BT

A single-neck round flask was charged with compound 8 (557 mg, 0.322 mmol) and chloroform (40 mL). After compound 8 dissolved, 5 mL trimethylamine solution in THF (2 M) was added into the reaction mixture and stirred at 55° C. for 16 h. After the reaction, the crude product was precipitated and attached to the bottom of the flask. The less colored solution was poured out and the solid precipitation was gently rinsed using chloroform five times. Then, the solid precipitation was dissolved using 40 mL methanol. 5 mL trimethylamine solution in methanol (3.2 M) was added into the reaction mixture and stirred at 55° C. for another 16 h. The solvent was removed via rotary evaporation and the solid was dried in a vacuum. The final product was obtained as a dark red solid (605 mg, 90% yield). ¹H NMR (500 MHz, DMSO-d₆) δ 8.29-8.18 (m, 4H), 7.81 (d, J=8.0 Hz, 4H), 7.75 (d, J=3.9 Hz, 2H), 7.68 (d, J=8.2 Hz, 4H), 7.27 (s, 4H), 6.96 (s, 4H), 4.12-4.00 (m, 8H), 3.95-3.87 (m, 4H), 3.37-3.27 (m, 12H), 3.09 (s, 54H), 1.84-1.62 (m, 24H), 1.58-1.47 (m, 12H), 1.42-1.28 (m, 12H). ¹³C NMR (126 MHz, DMSO) δ 153.57, 152.56, 145.89, 138.53, 138.02, 137.88, 133.42, 133.28, 129.93, 129.76, 128.01, 127.88, 126.56, 126.49, 125.68, 125.60, 106.00, 73.27, 69.11, 66.13, 60.82, 53.09, 30.40, 29.56, 26.54, 26.42, 26.02, 25.97, 23.01.

### Compound 10

Compound 6 (2.54 g, 2.64 mmol), compound 9 (0.31 g, 0.88 mmol), Pd(PPh₃)₂Cl₂ (25 mg, 0.035 mmol) were added to a round flask under the protection of nitrogen atmosphere. After purging using nitrogen, 10 mL dry toluene was added to the reaction mixture, which was then heated at 120° C. for 16 hours with stirring. After being cooled to room temperature, the reaction mixture was poured into water, and extracted with chloroform. The organic phase was dried over Na₂SO₄ and then the organic solvent was removed by a rotary evaporator. The crude product was purified with silica gel column chromatography using hexane and dichloromethane as eluent. The product was dissolved in chloroform and precipitated using methanol, and the precipitation was collected by filtration and washed using methanol. After being dried in a vacuum, the product was obtained as brown solid (0.69 g, 44% yield). ¹H NMR (500 MHz, Chloroform-d) δ 8.91 (d, J=4.1 Hz, 2H), 7.71 (d, J=8.0 Hz, 4H), 7.49 (d, J=8.5 Hz, 4H), 7.46 (d, J=4.2 Hz, 2H), 7.03 (d, J=16.2 Hz, 2H), 6.96 (d, J=16.1 Hz, 2H), 6.72 (s, 4H), 4.11-3.94 (m, 12H), 3.51-3.39 (m, 12H), 1.97-1.73 (m, 24H), 1.63-1.45 (m, 24H). ¹³C NMR (126 MHz, CDCl₃) δ 153.56, 151.49, 149.12, 138.58, 137.90, 137.43, 134.40, 133.48, 132.94, 129.36, 127.50, 127.23, 126.32, 124.54, 113.50, 105.58, 73.61, 69.26, 34.29, 34.17, 33.22, 33.11, 30.53, 29.66, 28.48, 28.32, 25.74, 25.72.

### Compound COE-BBT

A single-neck round flask was charged with compound 10 (196 mg, 0.110 mmol) and chloroform (20 mL). After compound 10 dissolved, 2 mL trimethylamine solution in THF (2 M) was added into the reaction mixture and stirred at 55° C. for 16 h. After the reaction, the crude product was precipitated and attached to the bottom of the flask. The less colored solution was poured out and the solid precipitation was gently rinsed using chloroform five times. Then, the solid precipitation was dissolved using 20 mL methanol. 2 mL trimethylamine solution in methanol (3.2 M) was added into the reaction mixture and stirred at 55° C. for another 16 h. The solvent was removed via rotary evaporation and the solid was dried in a vacuum. The final product was obtained as a brown solid (219 mg, 93% yield). ¹H NMR (400 MHz, DMSO-d₆) δ 9.13-9.04 (m, 2H), 7.97-7.83 (m, 6H), 7.77-7.66 (m, 4H), 7.29 (br, 4H), 6.97 (br, 4H), 4.12-3.99 (m, 8H), 3.95-3.85 (m, 4H), 3.39-3.23 (m, 12H), 3.09 (s, 54H), 1.86-1.63 (m, 24H), 1.59-1.47 (m, 12H), 1.43-1.28 (m, 12H). ¹³C NMR (126 MHz, DMSO) δ 153.57, 151.50, 148.78, 138.24, 138.06, 137.70, 134.93, 133.40, 133.29, 130.12, 128.08, 127.89, 126.72, 125.98, 113.24, 106.01, 73.27, 69.10, 66.11, 58.48, 55.32, 55.29, 55.26, 53.07, 30.41, 29.56, 26.55, 26.43, 26.02, 25.98, 23.00.

### Compound 12

The synthetical and purification procedures are similar to that for compound 5. The reactant tributyl(thiophen-2-yl)stannane (compound 4) was changed to (3,4-Ethylenedioxythien-2-yl)trimethylstannane (compound 11), but kept the feed ratio the same. The product was obtained as a colorless oil (5.49 g, 86% yield). ¹H NMR (500 MHz, Chloroform-d) δ 7.70 (d, J=8.4 Hz, 2H), 7.48 (d, J=8.5 Hz, 2H), 7.00 (d, J=16.2 Hz, 1H), 6.96 (d, J=16.2 Hz, 1H), 6.72 (s, 2H), 6.31 (s, 1H), 4.36-4.31 (m, 2H), 4.29-4.23 (m, 2H), 4.08-4.01 (m, 4H), 3.99-3.94 (m, 2H), 3.48-3.39 (m, 6H), 1.96-1.72 (m, 12H), 1.62-1.46 (m, 12H). ¹³C NMR (126 MHz, CDCl₃) δ 153.50, 142.61, 138.62, 138.36, 135.80, 133.13, 132.72, 128.67, 127.87, 126.93, 126.42, 117.68, 105.48, 98.04, 73.57, 69.22, 65.14, 64.79, 34.28, 34.16, 33.20, 33.07, 30.48, 29.61, 28.44, 28.27, 25.70, 25.69.

### Compound 13

The synthetical and purification procedures are similar to that for compound 6. The crude product was obtained as a colorless oil, which will be used in subsequent reactions without further purification (2.55 g, 94% yield). ¹H NMR (500 MHz, Chloroform-d) δ 7.72-7.68 (m, 2H), 7.49-7.45 (m, 2H), 6.99-6.95 (m, 2H), 6.71 (s, 2H), 4.34-4.20 (m, 4H), 4.07-3.94 (m, 6H), 3.47-3.39 (m, 6H), 1.96-1.79 (m, 12H), 1.60-1.45 (m, 12H), 0.38 (s, 9H).

### Compound 14

The synthetical and purification procedures are similar to that for compound 8. The product was obtained as dark green solid (431 mg, 40% yield). ¹H NMR (500 MHz, Chloroform-d) δ 7.85 (d, J=8.5 Hz, 4H), 7.53 (d, J=8.6 Hz, 4H), 7.04 (d, J=16.2 Hz, 2H), 6.98 (d, J=16.1 Hz, 2H), 6.73 (s, 4H), 4.53-4.46 (m, 4H), 4.39-4.32 (m, 4H), 4.09-3.95 (m, 12H), 3.48-3.40 (m, 12H), 1.96-1.72 (m, 24H), 1.60-1.48 (m, 24H). ¹³C NMR (126 MHz, CDCl₃) δ 153.51, 152.84, 142.17, 138.93, 138.45, 136.43, 133.07, 132.35, 129.01, 127.80, 126.97, 126.90, 122.06, 113.41, 109.52, 105.55, 73.57, 69.23, 65.03, 64.87, 34.26, 34.14, 33.19, 33.07, 30.48, 29.61, 28.44, 28.27, 25.70, 25.68.

### Compound COE-EDOT:

The synthetical and purification procedures are similar to that for compound COE-BBT. The product was obtained as dark green solid (181 mg, 93% yield). ¹H NMR (500 MHz, DMSO-d₆, 353K) δ 7.85-7.80 (m, 4H), 7.69-7.65 (m, 4H), 7.21 (br, 4H), 6.94 (br, 4H), 4.50 (br, 4H), 4.37 (br, 4H), 4.13-4.04 (m, 8H), 3.99-3.91 (m, 4H), 3.45-3.35 (m, 12H), 3.13 (s, 54H), 1.86-1.66 (m, 24H), 1.60-1.49 (m, 12H), 1.46-1.34 (m, 12H). ¹³C NMR (126 MHz, DMSO-d₆, 353K) δ 153.30, 152.36, 142.64, 139.29, 138.53, 136.80, 133.09, 132.01, 129.49, 127.77, 127.38, 126.53, 120.37, 112.86, 109.21, 106.52, 73.05, 69.29, 66.19, 65.34, 64.90, 53.04, 53.01, 30.05, 29.26, 26.23, 26.10, 25.58, 25.53, 22.70.

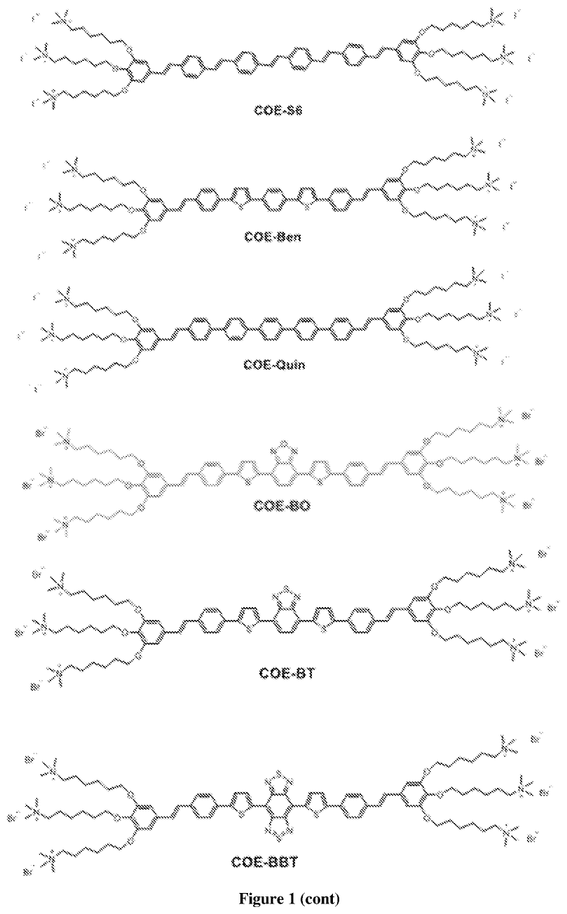

Other COE compounds (such as COE-BO ad COE-QX) can be similarly synthesised using the above protocols, their structures of which are shown in FIG. 1.

### Bacterial Cell Labelling and Detection in Flow Cytometry

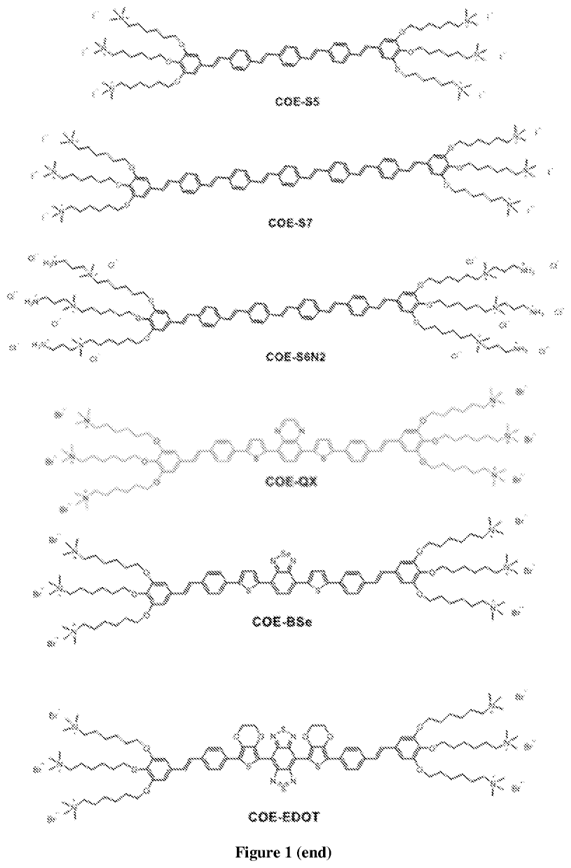

The application of COE dyes can be extended to label bacteria in flow cytometry measurements. As shown in FIG. 2, the Gram-positive bacteria methicillin-resistant Staphylococcus aureus (MRSA) and Gram-negative Escherichia coli (E. coli) in PBS were pre-mixed by volume using different ratios of 5:0, 4:1, 3:2, 2:3, 1:4, and 0:5. A COE dye that has Gram selectivity and can specially label the Gram-positive bacteria were employed to stain these bacteria mixtures. During the flow cytometry measurements, two isolated populations were seen. As the percentage of Gram-positive bacteria reduces, the population with positive signal decreases gradually and disappears when the bacteria mixture only contains the Gram-negative bacteria. These results only not prove that the compound is able to distinguish the bacterial ratio of each Gram-type, but also reveal that the compound can be used to label the bacterial samples in the flow cytometry.

### Labelling of Exosome and Detection Using Flow Cytometry

Exosome were prepared via the following protocol: Exosomes were purchased from ATCC, aliquoted and stored at −80° C. A single aliquot was removed from −80° C. and placed on ice. Exosomes were diluted 10 times in cell culture grade PBS. 8 μL of the exosome suspension was mixed with between 25-1 M of COE compound. This was placed on ice for 10-minutes before being diluted 100 times in PBS to an approximate final concentration of 6×10⁶ particles/mL of exosomes and 25-1 nM of COE.

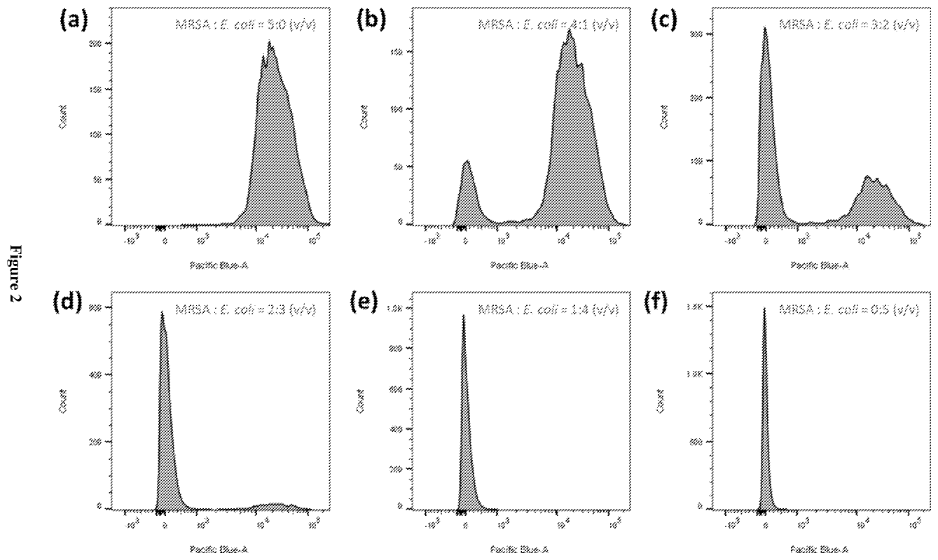

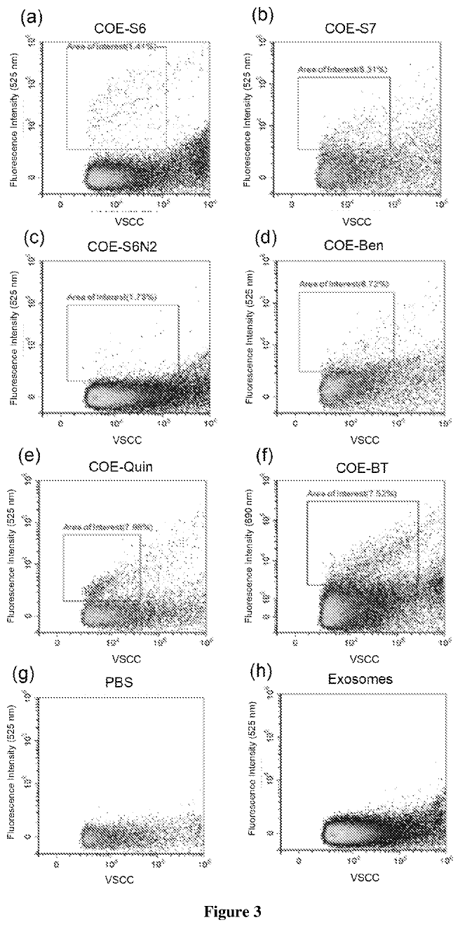

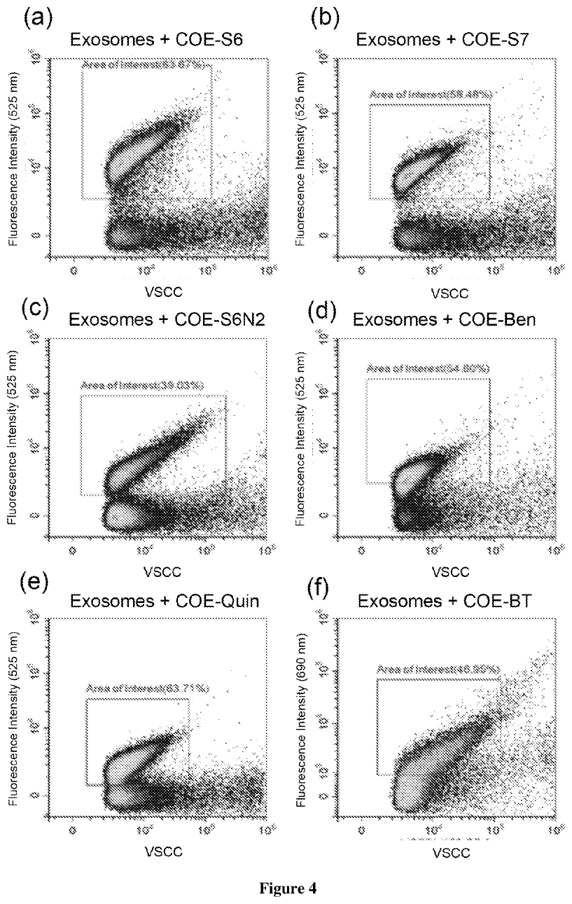

Samples were briefly vortexed and used for flow cytometry. Flow cytometry measurements were obtained by measuring the side scattering on the violet laser (x-axis) and by measuring fluorescent intensity (y-axis: λ_{excitation}=405, 488 nm λₑₘᵢₛₛᵢₒₙ=525, 690 nm). The experiments in FIGS. 3 and 4 demonstrates that exosomes can be successfully labelled and detected using COE compound. Using the COE compound, the exosomes can be stained with a low concentration of dye and can be used directly without additional purification. FIG. 1 shows the structures of the compounds.

TABLE 1
Exosomes used for flow cytometry

| | Cell | Cell | Number of | Particle | |
|---|---|---|---|---|---|
| Exosome name | line | type | Particles | size | Disease |
| HCT 116 Exosomes | HCT | Epithelial | 1.3 × 10⁹ | 50-200 | Colorectal |
| (ATCC ® CCL- | 116 | | particles/ | nm | Carcinoma |
| 247-EXM ™) | | | vial | | |

TABLE 2
Number of events measured by flow cytometry of exosomes.

| | | Area of | % of events |
|---|---|---|---|
| | All | Interest | in Area of |
| Sample | Events/μL | Events/μL | Interest |
| PBS | 595.59 | NA | NA |
| Exosome | 6115.79 | NA | NA |
| COE-S6 | 2909.37 | 41.08 | 1.41 |
| COE-S7 | 1137.64 | 60.45 | 5.31 |
| COE-S6N2 | 4334.17 | 74.96 | 1.73 |
| COE-Ben | 1480.33 | 99.48 | 6.72 |
| COE-Quin | 1438.47 | 114.78 | 7.98 |
| COE-BT | 3424.14 | 257.39 | 7.52 |
| Exosome + COE-S6 | 5113.31 | 3255.41 | 63.67 |
| Exosome + COE-S7 | 5143.77 | 3008.20 | 58.48 |
| Exosome + COE-S6N2 | 6173.19 | 2409.60 | 39.03 |
| Exosome + COE-Ben | 6071.19 | 3314.72 | 54.69 |
| Exosome + COE-Quin | 6088.97 | 3878.99 | 63.71 |
| Exosome + COE-BT | 3813.49 | 1790.48 | 46.95 |

In another example, exosomes isolated from SW480 colon cancer cell lines and purified using size exclusion chromatography were kindly provided by NanoFCM (UK). The labelling and flow cytometry analysis were also carried out by the trained technicians at NanoFCM (UK). Briefly, the exosomes were labelled at a particle concentration of approximate 10e¹⁰ particles per mL, in which 9 μL of the sample was added with 1 μL of 10 μM COE dye solutions or PKH-26 (in diluent C) to achieve a final 1 μM staining concentration. The mixture was left to incubate at room temperature for 30 min and then diluted 100 times using PBS before analysis on the NanoAnalyzer (NanoFCM). Detection of the exosomes was carried out on the small threshold settings (68-155 S16M-Exo) and analysis of the labelled population was done on the PC5 channel (Ex488/Em670). Dye control samples were prepared by replacing the 9 μL exosome solution with PBS, followed by the same steps as the exosome samples for flow analysis. The results were validated through at least two replicates. In a separate experiment, the labelled exosomes were also purified of excess dyes using standard ultracentrifugation methods before analyzing as per described above.

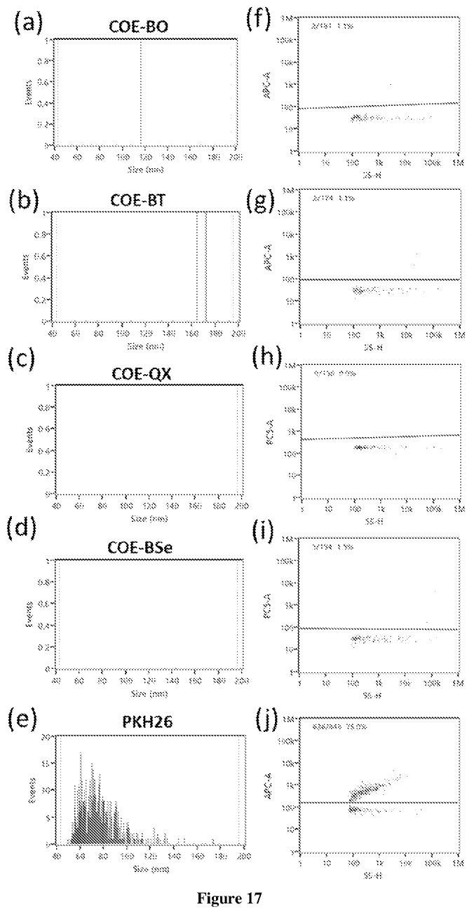

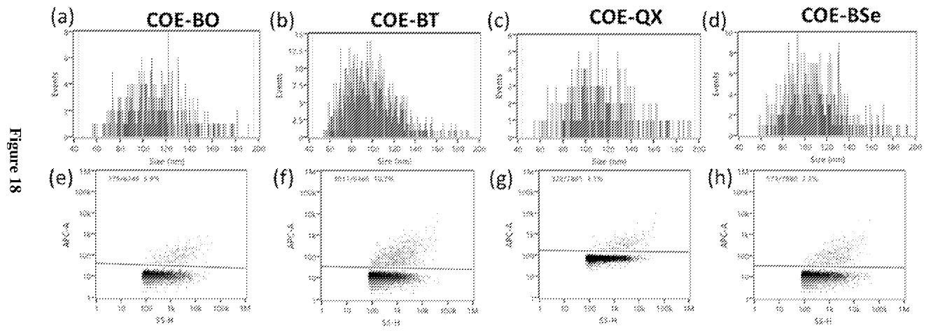

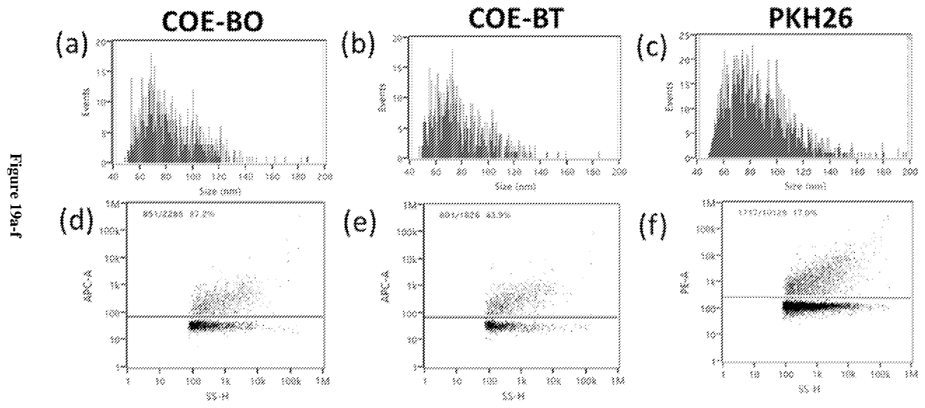

FIG. 17 shows (a-e) particle size distribution plots of dye only controls for COE-BO, COE-BT, COE-QX, COE-BSe and PKH26 (10 μM) and (f-j) their corresponding gated dot plots. FIG. 18 shows (a-d) particle size distribution plots of COE-labelled SW480 exosomes (10 μM) and (e-h) their corresponding gated dot plots. Table 3 and 4 summarises the results of COE-only buffer controls and COE labelled exosomes when analysed using NanoAnalyzer.

TABLE 3
Number of particle counts in samples of dye
only controls analyzed on NanoAnalyzer.

10 uM

| Dye Controls | total | positive |
|---|---|---|
| COE-BO | 181 | 2 |
| COE-BT | 174 | 2 |
| COE-QX | 220 | 3 |
| COE-BSe | 194 | 3 |
| PKH-26 | 637 | 636 |

TABLE 4
Percentage positive events in COE-labelled
SW480 exosomes analyzed on NanoAnalyzer.

| | Percentage + |
|---|---|
| | (N = 2) |
| Stained EVs | 10 uM |
| COE-BO | 66.55% |
| COE-BT | 73.65% |
| COE-QX | 63.40% |
| COE-BSe | 70.05% |

FIG. 19 and Table 5 shows the results after purifying excess COE compounds from COE-labelled SW480 exosomes.

TABLE 5
Percentage positive events after purifying excess dyes
using ultracentrifugation using COE-labelled SW480
exosomes (10 and 20 μM) analyzed on NanoAnalyzer.

% Positive for dye

| | 10 uM | 20 uM |
|---|---|---|
| COE-BO | 43.10% | 47.60% |
| COE-BT | 52.90% | 65.30% |
| PKH-26 | 22.00% | 19.90% |

### Transmission Electron Microscopy of Exosomes (TEM)

Samples prepared for flow cytometry or collected from fluorescence activated cell sorting (FACS) were submerged in liquid nitrogen and lyophilized. The powder was the resuspended in 50 μL of cold MiliQ water. Glutaraldehyde was added to a final concentration of 2.5%, 10 μL of sample was added to a freshly glow discharge 200 mesh Cu Carbon/Formvar grid and allowed to adhere for 10 min. The excess liquid was wicked with filter paper and the grid was washed two times with MiliQ water. The grid was stained for 1 minute with 2.5% Gadolinium triacetate stain and then excess liquid was wicked with filter paper. Samples were imaged with a Tecnai G2 at 100 kV at 30,000× magnification. FIG. 5 shows TEM micrographs of (a) exosomes, exosomes labeled with COE-BT and c) exosomes labeled with COE-S6 collected after FACS.

### Red Blood Cells (RBCs) Labelling and Detection Using Flow Cytometry

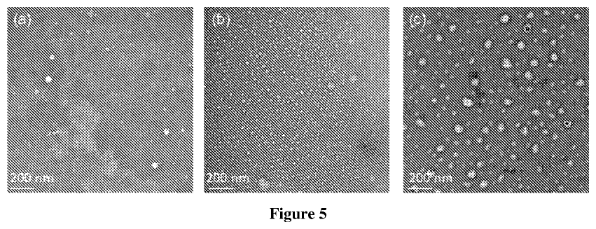

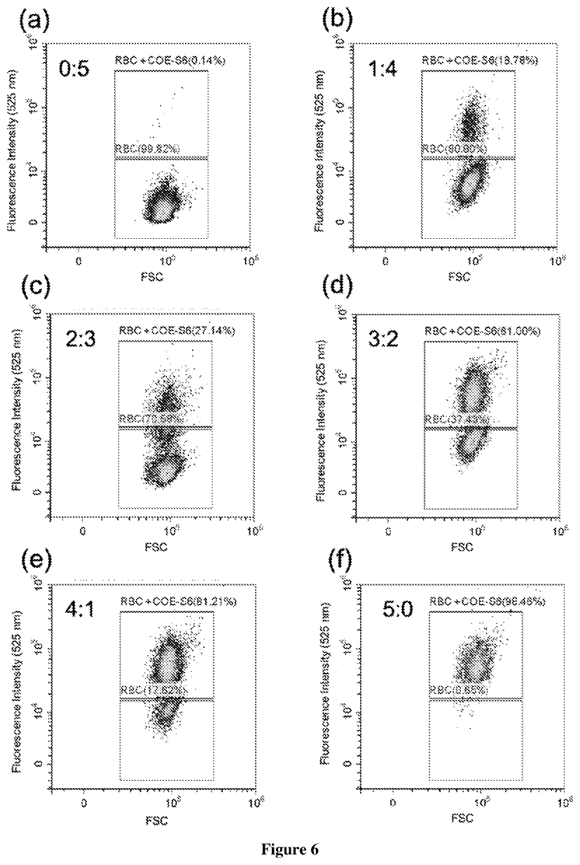

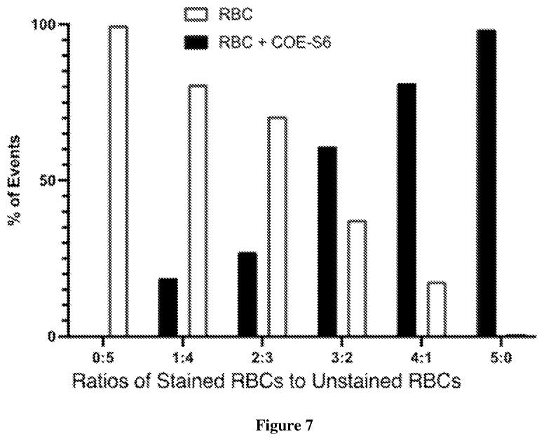

COE compounds can be used to label RBCs for flow cell cytometry as shown in FIG. 6. RBCs (isolated from Bovine whole blood, Innovative Research, Inc.) were diluted 10 times from stock into PBS and centrifuged at 3,000 RPM for 5 minutes. The supernatant was removed, and the pellet was resuspended in PBS. This was repeated for a total of 3 times. The final pellet was resuspended into PBS to be 1% by volume. 10 M COE compound was added to the RBC suspension and was allowed to incubate for 20 min at RT. The RBC COE compound mixture was then centrifuged again at 3,000 RPM for 5 min. The supernatant was removed and the pellet resuspended in fresh PBS. COE compound stained and unstained RBCs were respectively mixed at various ratios by volume of 0:5, 1:4, 2:3, 3:2, 4:1 and 5:0. The resulting RBC mixtures were diluted 100 times in PBS for flow cytometry experiments. Flow cytometry measurements were obtained by measuring the forward scattering (x-axis) and by measuring fluorescent intensity ((y-axis: λ_{excitation}=405 nm λₑₘᵢₛₛᵢₒₙ=525 nm). FIG. 6 demonstrates that two distinct populations can be seen for COE compound labelled and unlabelled RBCs. As the ratio of RBCs stained with COE compound increases, the percentage of the population with a higher fluorescent intensity also increases indicating successful labelling of the RBCs with COE compound and successful detection via flow cytometry (FIG. 6).

TABLE 6
Number of events measured in the gated areas of COE-S6 stained
RBCs to unstained RBCs at various ratios by flow cytometry

| Ratio of | RBC + | | |
|---|---|---|---|
| Stained to | COE-S6 | RBC | total |
| Unstained RBCs | Events/μL | Events/μL | events/μL |
| 0:5 | 1.9 | 1318.27 | 1320 |
| 1:4 | 207.2 | 891.58 | 1098 |
| 2:3 | 312.27 | 812.03 | 1124 |
| 3:2 | 767.09 | 470.71 | 1237 |
| 4:1 | 1039.71 | 225.64 | 1265 |
| 5:0 | 450.48 | 2.98 | 453 |

### Mammalian Cell Labelling and Detection Using Flow Cytometry

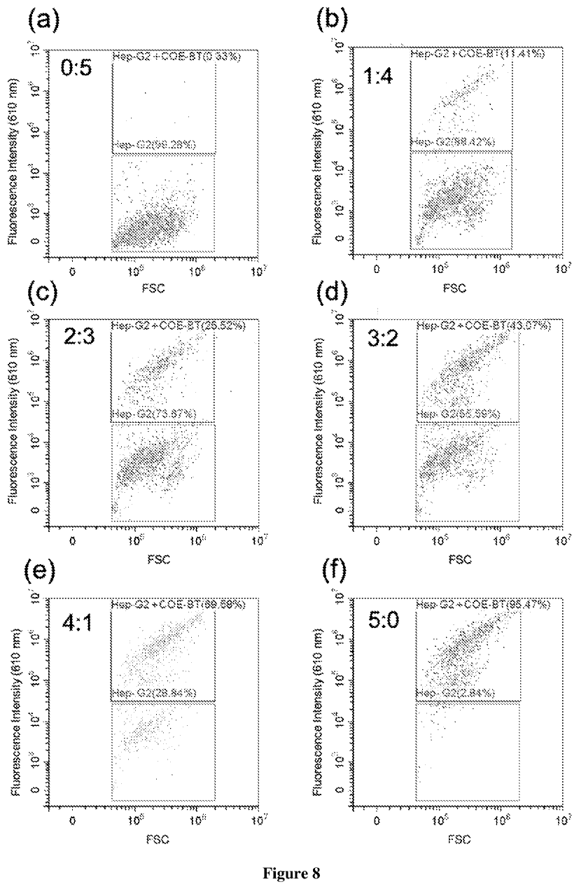

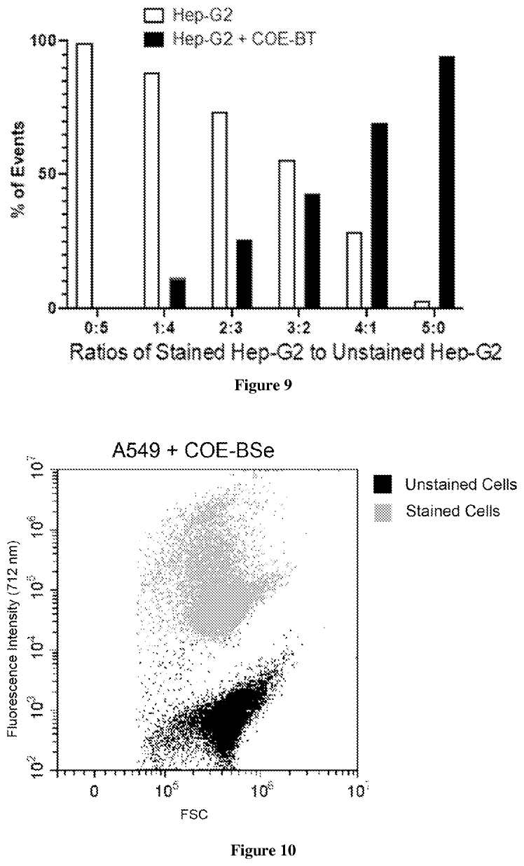

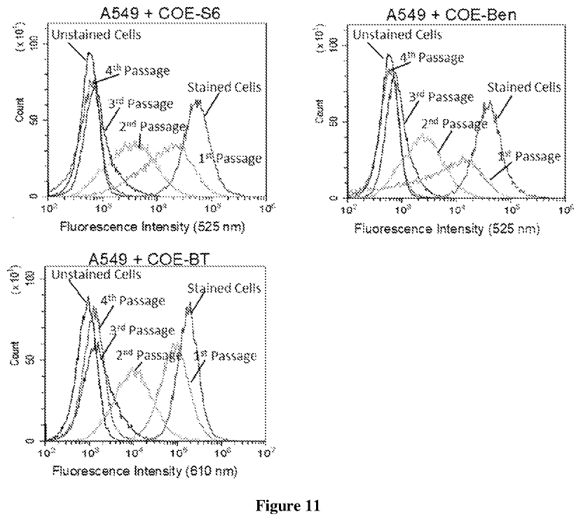

COEs can be used to label mammalian cells for flow cell cytometry as shown in FIGS. 8-11. Hep-G2 and A549 (ATCC HB-8065, CCL-185) were purchased from ATCC. Upon receiving the frozen cells, cells were thawed by gentle agitation in a 37° C. water bath and were later transferred to a centrifuge tube containing 9 mL prewarmed culture medium of DMEM+10% FBS. The tube was later centrifuged at 200×g for 5 min. The supernatant was discarded, and the cell pellet was resuspended with culture media in a culture flask. Cells were incubated at 37° C. with 5% CO₂. When cell confluency reached around 90%, cells were lifted with 1× Trypsin and neutralized by the same volume of culture media. After centrifuging at 200×g for 5 min, the supernatant was discarded and the cell pellet was resuspended with warmed PBS containing 5-10 μM COE. The suspension was allowed to incubate for 20 min at RT before centrifuging at 1000 rpm for 5 min to remove excess COE compound. The supernatant was discarded, and the pellet was then resuspended in warmed PBS. COE compound stained and unstained cells were respectively mixed at various ratios by volume of 0:5, 1:4, 2:3, 3:2, 4:1 and 5:0. The resulting suspensions were used directly for flow cytometry experiments. Flow cytometry measurements were obtained by measuring the forward scattering (x-axis) and by measuring fluorescent intensity (y-axis: λ_{excitation}=561 nm, λₑₘᵢₛₛᵢₒₙ=610 nm). FIG. 8 demonstrates that two distinct populations can be seen for COE labelled and unlabelled cells. As the ratio of cells stained with each COE compound increases, the percentage of the population with a higher fluorescent intensity also increases indicating successful labelling of the cells with COE and successful detection via flow cytometry (FIG. 8 and FIG. 10). FIG. 9 shows percentage of flow cytometry events occurring in the gated areas of compound of Formula (I) stained Hep-G2 to unstained Hep-G2 at various ratios. FIG. 11 shows flow cytometry measurements of A549 cells labeled with 5 μM COE-BSe. Flow cytometry measurements were obtained by measuring the forward scattering (x-axis) and by measuring fluorescent intensity (y-axis: λ_{excitation}=638 nm, λₑₘᵢₛₛᵢₒₙ=712 nm). Confluent cells were stained with 5 μM COE in PBS at RT for 20 min. The COE solution was aspirated and the confluent cells were washed with fresh PBS. Cells were lifted with 1× Trypsin and neutralized by the same volume of culture media. After centrifuging at 200×g for 5 min, the supernatant was discarded, and the cell pellet was resuspended with warmed PBS and used for flow cytometry. 20% of the COE labelled cell suspension was seeded and allowed to reach 90% confluency. Cells were harvested and seeded then used for flow cytometry without additional COE added for 4 passages. (FIG. 11)

TABLE 7
Number of events measured in the gated areas of stained Hep-G2 cells
to unstained Hep-G2 cells at various ratios by flow cytometry

| Ratio of | Hep-G2 | Hep-G2 | |
|---|---|---|---|
| Stained to | unstained | stained | Total |
| Unstained Hep-G2 | events/μL | events/μL | events/μL |
| 0:5 (COE-BT) | 3024.06 | 10.05 | 3046 |
| 1:5 (COE-BT) | 2496.09 | 322.10 | 2823 |
| 2:3 (COE-BT) | 2171.05 | 752.07 | 2947 |
| 3:2 (COE-BT) | 1577.08 | 1221.90 | 2837 |
| 4:1 (COE-BT) | 564.97 | 1363.07 | 1959 |
| 5:0 (COE-BT) | 57.08 | 1898.85 | 2010 |

### Fluorescent Microscopy of Mammalian Cells

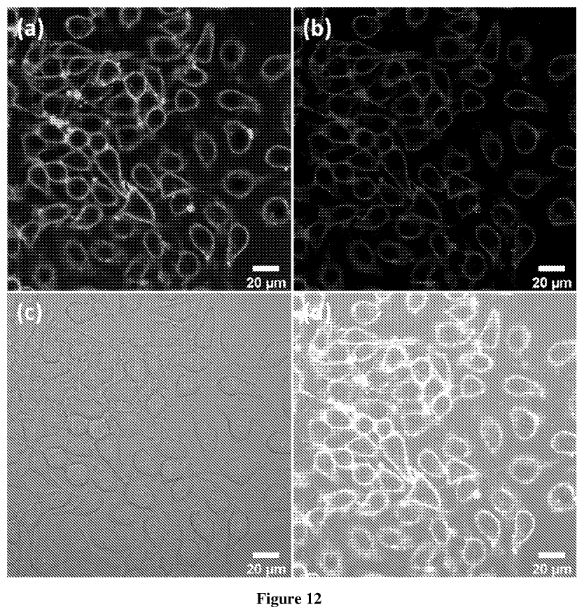

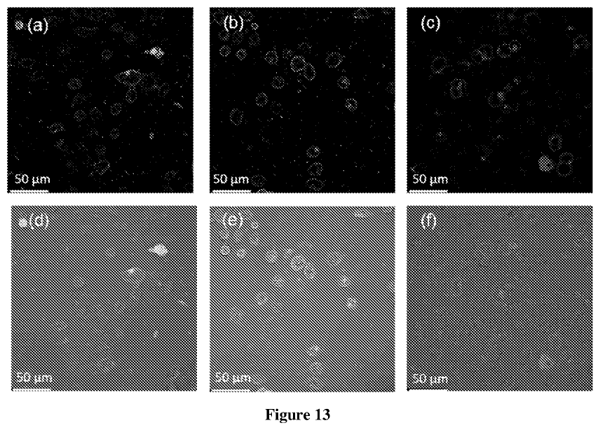

The above experiments have confirmed that COE dye can be used to label the extracellular vesicles (including exosomes), bacteria, RBCs, and mammalian cells in flow cytometry. Among these samples, they all contain a lipid bilayer structure in their membrane. To further identify that the binding target of COE dyes is the lipid membrane, colocalization between COE compound and a commercially available membrane dye FM 4-64 was performed using confocal microscopy. As shown in FIG. 12, the mammalian Hep-G2 cells were stained by both 4 μM COE compound (green channel) and 4 μM FM 4-64 (red channel), and clear staining patterns delineating the cellular membrane were observed by collect the emission from COE compound in the range of 450-490 nm after excitation at 405 nm. The well-matched colocation between COE compound and FM 4-64 proves that the binding target of COE dye is the lipid membrane. A549 cells were stained with 5 μM COE compound in PBS and were incubated at 37° C. with 5% CO₂ for 1 hour before visualising on a fluorescent microscope. (FIG. 13)

Evaluation of COE Intercalation into a Lipid Bilayer of Using a Fluorimeter

Liposomes were prepared using the protocol as follows. Phospholipids (ex. POPC, POPE, and POPG) in chloroform solution were mixed to a proper molar ratio and dried under a gentle stream of nitrogen. The dried lipids were further desiccated in a vacuum overnight to obtain a thin lipid film. For preparing small unilamellar vesicles (SUVs), rehydration of the dried film was carried out by adding phosphate buffered saline (PBS) to a concentration of 5 mg mL-1, followed by incubation at 35° C. for 2 h under constant stirring at ˜300 rpm. Then, the vesicles were extruded using a 100 nm membrane at 45° C. 21 times to obtain SUV samples. Vesicles were kept at 4° C. until further use.

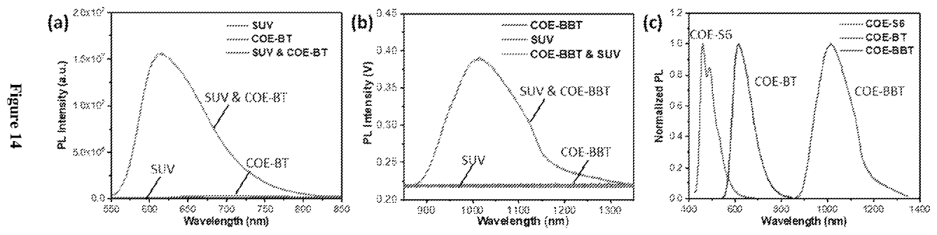

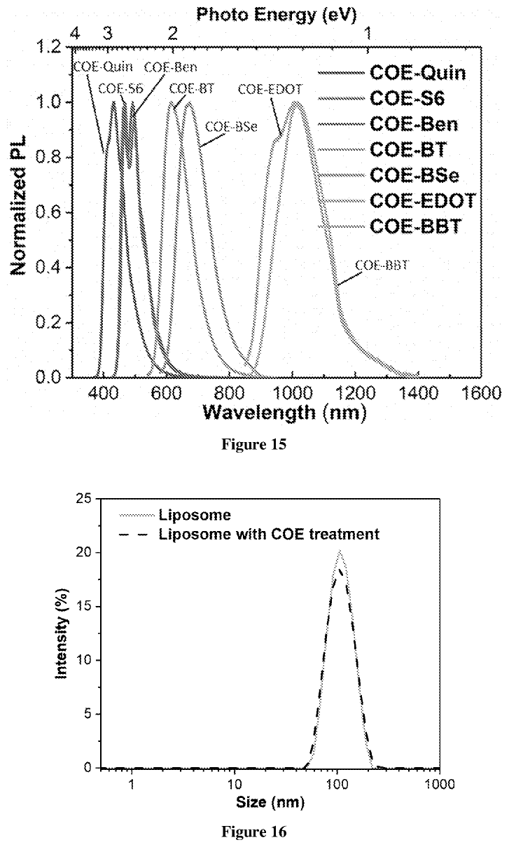

The emission of COEs will enhance significantly after the COEs intercalate into the lipid bilayers. Hence, the free dyes in the aqueous phase will be weakly emissive and will yield less background, which is ideal for obtaining a high signal-to-noise ratio. As shown in FIG. 14, both SUVs (1 mg/mL) and COE-BT (10 M) in PBS exhibit weak emission (λ_{excitation}=520 nm). When mixing the SUVs with COE-BT, the COE will intercalate into the lipid bilayers. This is spontaneously driven by the electrostatic and hydrophobic interactions between the COEs and the lipids. As a result, a significant emission enhancement is observed. For example, relative to the COE-BT solution, the emission intensity of the SUV and COE-BT complexes increases more than 400 times between 600 to 630 nm. The same phenomenon is seen for COE-BBT as well (FIGS. 14 & 15). By adjusting different chemical structures, the COEs can achieve different emission wavelengths, ranging from “green”, “red”, to even “infrared” (FIGS. 14 & 15). The various color choices confer COE dyes with high compatibility in using multiple dyes concurrently.

### Evaluation of COE Addition on Liposome Size and Uniformity

To identify whether COE treatment will alter the morphology of liposomes (e.g., inducing the fusion or fission of liposomes), dynamic light scattering (DLS) measurements with a lipid SUV model were employed (FIG. 16). The SUVs were obtained after extrusion using a 100 nm membrane. The Z-average size of 104.5±29.45 nm with a PDI of 0.048 was observed for the 1 mg/mL SUV samples during the DLS measurement. After addition of 10 μM COE-BBT into the 1 mg/mL SUV samples, the Z-average size is 104.8±32.65 nm with a PDI of 0.054. This DLS results show that COE addition will not alter the liposome morphology.

The results show that can the compounds of the present invention be used across a wide range of samples including, exosomes, red blood cells, mammalian cells and bacterial cells. The compounds show a greater labelling efficiency and sensitivity through its increased fluorescence intensity. These results reveal that COEs can be used as fluorescent labels in flow cytometry that is predominantly reliant on its interaction with the lipid bilayer. The tunability of COE molecular structure and ease of synthesis offer a wide range of possibilities and flexibility in tailoring to emission detection needs.

COE Labelled Exosomes and their Uptake into Mammalian Cells Using Imaging Flow Cytometry

A549 cells in DMEM media containing 10% FBS were seeded at a concentration of 1×10⁵ cells mL-1 in a 6-well plate and allowed to adhere overnight before experiments. At the next day, 4.5 mL of 10 μg mL-1 PC-3 exosomes or controls (PBS only) were mixed with COE-Ben to a final dye concentration of 1 μM or DiD to a final dye concentration of 2.5 μM. Samples were incubated at RT for 1 hour in the dark. Samples were washed by filtration using a 100 KDa cutoff centrifugal filter unit (Amicon® Ultra) and centrifuged at 2000×g for 15 min to remove the free dye. The concentrated samples were recovered and resuspended in FBS free DMEM media, which were divided into 3 aliquots. The cells in microplates were rinsed using FBS free DMEM media before stained exosomes or controls (residual dye solutions) were added at different time points, like 2, 4, and 8 hours before harvesting the cells. After exosome or dye uptake, cells were washed with warmed PBS (37° C.) and 1× Trypsin-EDTA was added. The cells were left to incubate in the 5% CO₂ chamber at 37° C. for up to 5 min. DMEM containing 10% FBS was added into microplates and cells were centrifuged at 200×g for 4 min. Supernatant was removed and cells were resuspended in DMEM media (contains 10% FBS) and taken for imaging flow cytometry using an Amnis ImageStreamX Mk II Imaging Flow Cytometer. The collection gate was set using the area vs aspect ratio of the brightfield channel to select for intact cell events only. For the COE-Ben channel (i.e., Ch02), the cells were excited using 405 nm laser and the emission was collected in the range of 505-560 nm. For the DiD channel (i.e., Ch05), the cells were excited using 638 nm laser and the emission was collected in the range of 642-745 nm. The brightfield area of the whole cell was employed as the indication of size, i.e. the M04 or M01 channel. Data was processed using IDEAS v6.3. A gradient RMS (root-mean-square) on brightfield channel was applied to exclude cells out of focus. The flow cytometry results were presented using the M04 channel (size) versus the Ch02 channel (COE-Ben) or Ch05 channel (DiD). Gates were set according to the unstained control samples. The built-in “spot count algorithm” function in IDEAS 6.3 software was used to count the number of bright spots within the cell and used as a metric for exosome uptake. At least 1000 cells were statistically counted in each group.

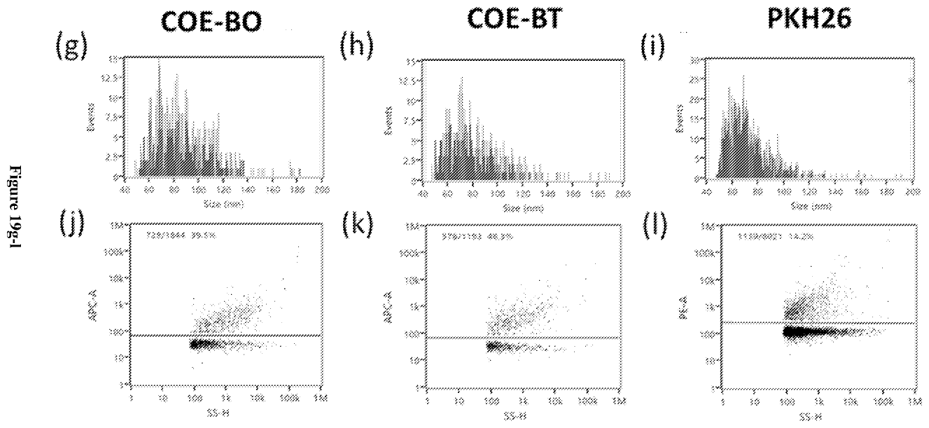

FIG. 20 shows (a) representative imaging flow cytometry images of A549 cells stained by the COE-Ben-stained exosomes for different treatment times, and (b-d) their corresponding flow cytometric analysis. (e) Mean spot count of COE-Ben or DiD probe (1,1′-dioctadecyl-3,3,3′,3′-tetramethylindodicarbocyanine, 4-chlorobenzenesulfonate) in each cell. (f) Representative imaging flow cytometry images of A549 cells after stained by the residue of COE-Ben or DiD dye after washing using ultrafiltration, and (g-h) their corresponding flow cytometric analysis. In the imaging flow cytometry, the COE-Ben channel was excited using 405 nm and the emission was collected in the range of 505-560 nm, the DiD channel was excited using 638 nm and the emission was collected in the range of 642-745 nm, the brightfield area of the whole cell was employed as the indication of size.

### Labelling Pre-Stained Exosomes Using COE

To distinguish between exosomes produced by the A549 cells and the exosomes that originate from serum, a new cell culture media (CCM-Exo-Dep) was prepared for the exosome production by supplementing DMEM with exosome-depleted fetal bovine serum (Systems Biosciences, Catalogue: EXO-FBS-250A-1). The A549 cells were seeded at 5×10⁵ cells mL⁻¹ in 10 mL of CCM-Exo-Dep within T75 flasks and left to adhere for 1 hour before 2 μM COE-BT was introduced into the cell culture. The cells were left to incubate for at least 24 hours before the cell culture supernatant was recovered for the first round of exosome production and stored at 4° C. before purification. Without passaging the labelled cells, fresh 10 mL CCM-Exo-Dep was added to the cells and left to incubate for at least another 24 hours. The cell culture supernatant for the second round of exosome production was then recovered after 24 hours. The collected cell cultures were first spun at 10,000×g for 30 min at room temperature to remove any cellular debris. Exosome isolation was then carried out via ultracentrifugation (Optima™ XPN-100, Beckman Coulter) at 100,000×g for 1 hour at 4° C. The supernatant was carefully removed and the bottom of the centrifuge tube, at which the pellet is expected to be located, was rinsed with PBS three times. The exosomes were pelleted again under the same conditions after washing and then resuspended in 100 μL of PBS. Aliquots of the purified exosomes were stored at −80° C. before downstream analysis (flow cytometry or TEM).

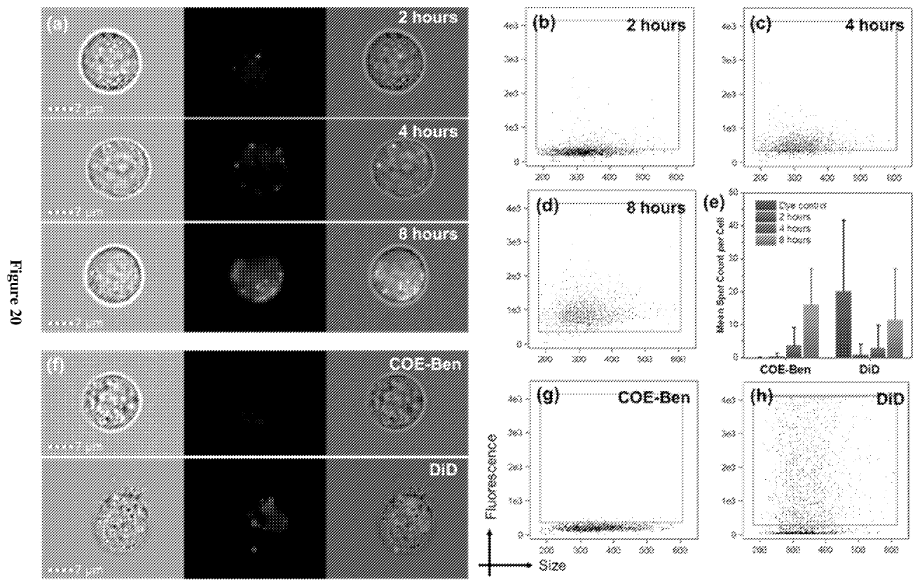

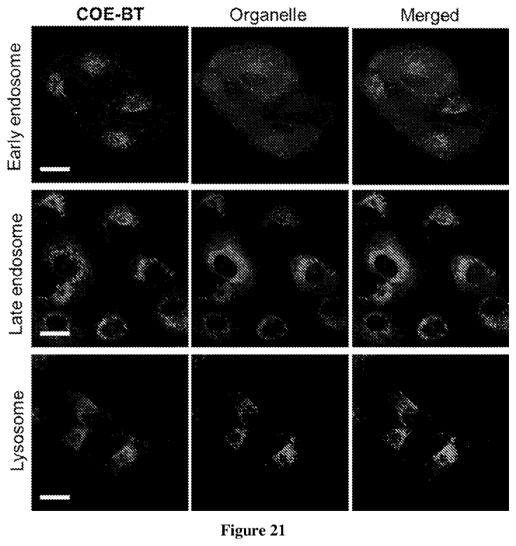

FIG. 21 shows colocalization micrographs of A549 cells after incubation with 2 μM COE-BT, and then stained by Early or Late Endosomes-GFP reagent (BacMam 2.0) or 100 nM lysosome-specific dye LysoTracker® Green DND-26. The COE-BT channel (represented in green) was observed by excitation at 561 nm and collecting the emission in the range of 570-620 nm. The GFP (green fluorescent protein) and LysoTracker Green channels (represented in red) were observed by excitation at 488 nm and collecting the emission in the range of 500-540 nm. Pearson's correlation coefficient (R) as the metric of colocalization was analyzed using ImageJ.

The exosome sample was analysed using transmission electron microscopy (TEM). Briefly, exosomes, obtained from pre-stained A549 cells were fixed using 2.5% glutaraldehyde in PBS for 30 min at room temperature. 10 μL of exosome solutions were added onto a freshly glow-discharged formvar carbon coated 200 mesh copper EM grids for 15 min. Samples were blotted using filter paper and washed 3 times with MiliQ water. Samples were negative stained with 2% gadolinium acetate for 30 s. Samples were blotted with filter paper and stored in grid box inside a desiccator vacuum chamber overnight before imaging on a FEI TENCAI G2 instrument using 80 kV.

FIG. 22 shows (a) transmission electron microscopy image of EVs secreted by COE-BT-stained A549 cells. (b) Flow cytometry analysis of EVs secreted by COE-BT-stained A549 cells after the first 24-hour incubation. The excitation and emission wavelengths are 488/690 nm for the B690 channel.

Lack of Nanoparticle Formation with COE

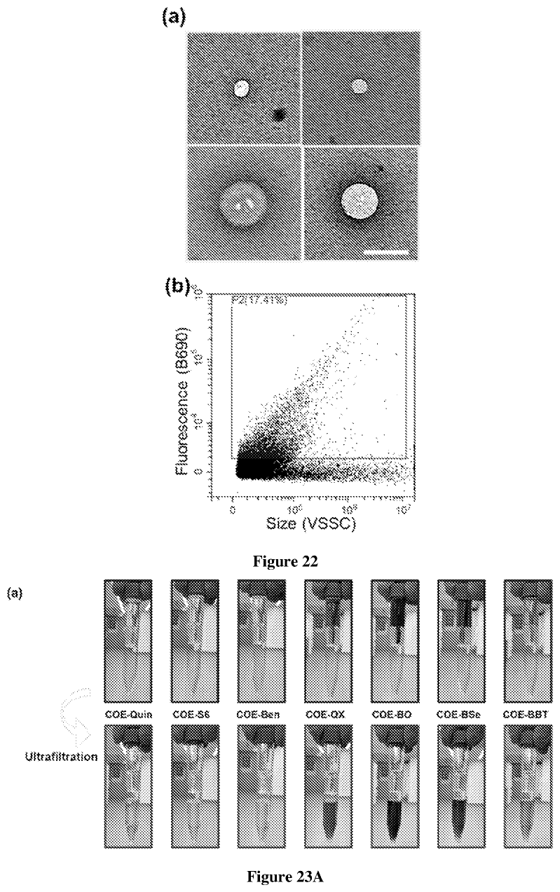

FIG. 23 shows (a) photographs and (b) absorption spectra of 50 μM COEs or DiR (1,1′-dioctadecyl-3,3,3′,3′-tetramethylindotricarbocyanine iodide) solutions in PBS before and after ultrafiltration using 100K MWCO protein concentrator tubes (Pierce™, Thermo Scientific™) at 4000 relative centrifugal force. The slight reduce in the absorption spectra for the COE samples is probably due to the unspecific binding between the positively charged COEs and polyethersulfone-based ultrafiltration membrane.

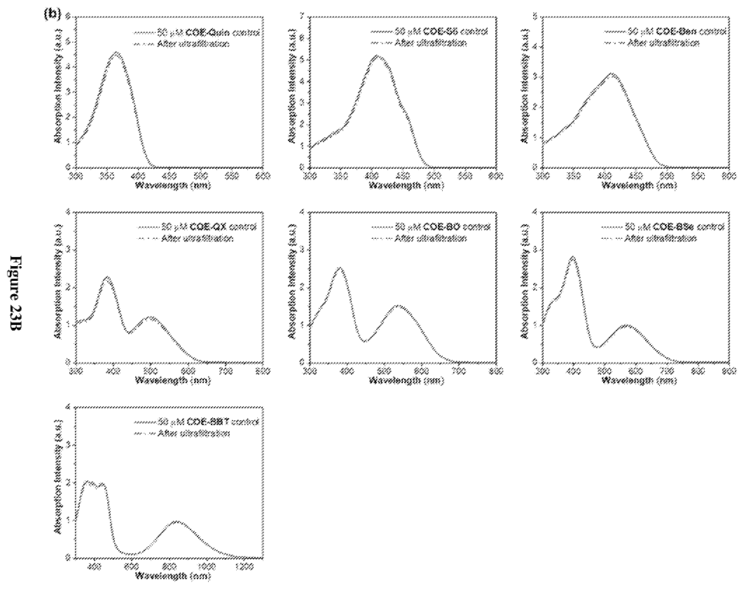

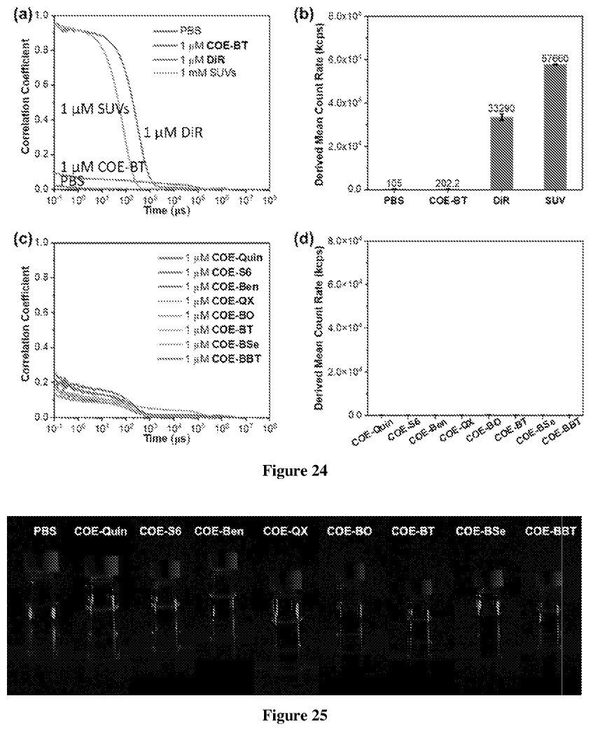

FIG. 24 shows (a) correlation coefficient curves of neat PBS, 1 μM COE-BT or 1 μM DiR or 1 mM SUVs in PBS as measured by dynamic light scattering (DLS). (b) DLS measured derived mean count rate of neat PBS, or 1 μM COE-BT, or 1 μM DiR, or 1 mM SUVs in PBS; the experiments were performed with five replicates. (c) Correlation coefficient curves of 1 μM other COEs in PBS as measured by DLS. (d) DLS measured derived mean count rate of 1 μM other COEs in PBS; the experiments were performed with five replicates.

FIG. 25 shows photographs of Tyndall effects of neat PBS or 10 μM COEs in PBS after being illuminated using a red laser pointer.

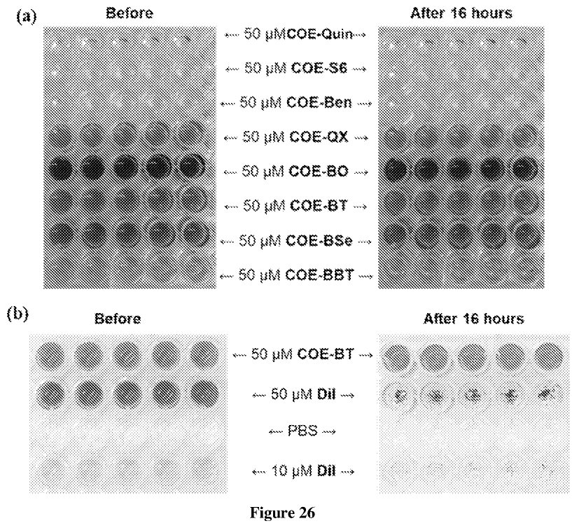

FIG. 26 shows (a) photographs of 200 μL COE solutions in PBS in a 96-well microplate (Costar® polystyrene-based, Ref: 3599) before and after statically setting for 16 hours at room temperature. (b) Photographs of 200 μL dye solution in PBS in a 96-well microplate (Costar® polystyrene-based, Ref: 3599) before and after statically setting for 16 hours at room temperature. The COE solution was obtained by diluted from 1 mM stock solution in PBS. The DiI solution was obtained by dispersed DiI DMSO solution (1 mM) into PBS with vigorous mixing. Same volume of PBS was used as negative controls.

### Stability of COE in Labelled Vesicles Using Flow Cytometry

Liposomes, comprising 15% POPG and 85% POPC, were prepared as 5 mg/mL (or 6.564 mM) stock in PBS as described before. The liposomes were extruded through 200 nm membrane filters and further characterized by DLS to be approximately 140 nm in size. Separate aliquots of the stock liposomes were then diluted to 1 mM and treated with 20 μM COE-Ben (or DiO) and 40 μM COE-BT (or DiD) in PBS respectively, under gentle heating to 60° C. for 30 min. The labelled liposomes were then stored at 4° C. before being used for flow cytometry experiments. To demonstrate the stability of the dyes in the liposomes after intercalation, two separate working solutions of COE-Ben labelled liposomes and COE-BT liposomes were first normalized to the same particle concentration of 107 particles per mL. These two working solutions were then mixed in varying ratios of 0:5, 1:4, 2:3, 3:2, 4:1 and 5:0 and the mixtures were allowed to incubate at room temperature for 1 hour. The undiluted mixtures were then analyzed on the CytoFLEX LX (Beckman Coulter) where detection is triggered on the violet side scatter (VSSC>4000). With reference to unstained liposomes, gating was done on both the V525 channel (Ex405/Em525) and B690 channel (Ex488/Em690) for COE-Ben labelled liposomes and COE-BT labelled liposomes respectively. The percentage of events in the dual-positive region of the dot plot was used to analyze for liposomes that may contain both COE-Ben and COE-BT dyes, suggesting the transfer of dyes from one population to another. Further, the samples were left to incubate at room temperature overnight (24 hour) before doing the same flow cytometry analysis to check for the stability of the dyes and the extent of cross-over spillage of the dyes. The experiment was carried out in duplicates.

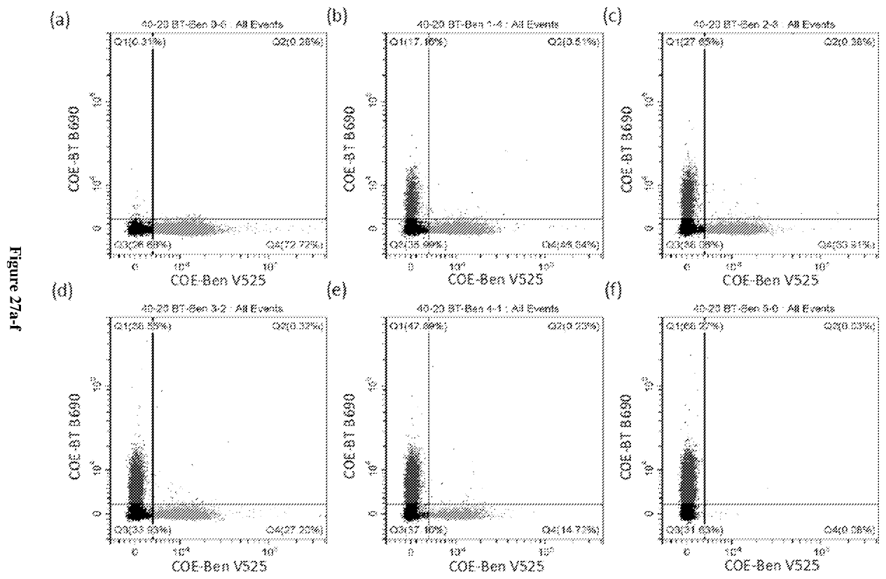

FIG. 27 shows (a-f) flow cytometry measurements for mixture of COE-BT-stained and COE-Ben-stained SUVs (130 nm) in PBS with different mixing ratio and left incubated for 1 h and (g-1) 24 h analyzed on Cytoflex.

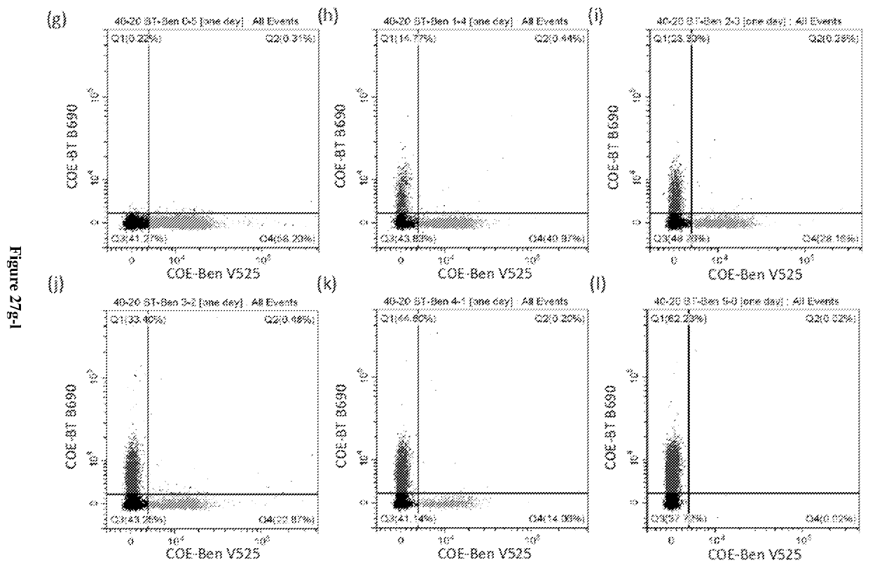

FIG. 28 shows SUV population percentages from FIG. 27 in different gates after mixing for (a) 1 or (b) 24 hours.

### Labelling Liposomes of Different Sizes Using COE

POPC in chloroform solution was mixed with 0.5 mol % of COE-Ben or COE-BT chloroform solution, and then dried under a gentle stream of argon in a glass vial. The dried lipid was further desiccated in a vacuum overnight to obtain a thin lipid film. To prepare the small unilamellar vesicles (SUVs), rehydration of the dried film was carried out by adding PBS buffer (phosphate-buffered saline) to a concentration of 5 mg mL⁻¹, followed by incubation at 45° C. for 2 hours under constant stirring at ≈300 rpm. Then, the vesicles were extruded using 100, 200, 400 and 800 nm membrane at 45° C. for 21 times to obtain SUV samples. The labelled liposomes were then diluted to 10′ particles per mL before analysing on the Cytoflex.

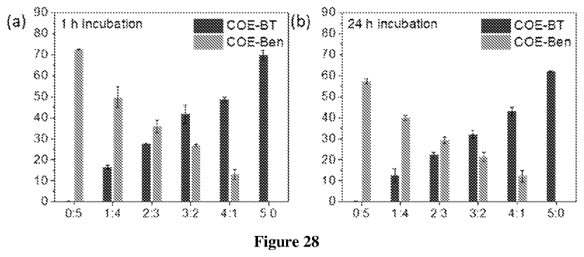

FIG. 29 shows dot plot profiles of dye-positive events in which POPC liposomes of 100, 200, 400 and 800 nm size, labelled with 0.5 mol % COE-Ben (a-d) and COE-BT (e-f) were analyzed on the Cytoflex.

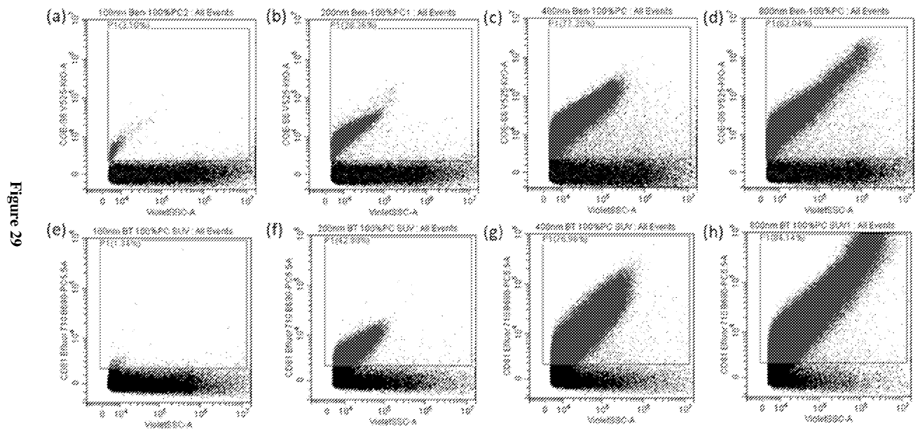

FIG. 30 shows (a) confocal micrographs of 6.25 mg mL⁻¹ LMVs (large multilamellar vesicles) after stained by 15 μM COE-Ben and 15 μM FM 4-64 in PBS for 30 minutes at room temperature. The mixture was diluted 5 times using PBS before confocal imaging. The COE-Ben fluorescent channel was observed by excitation at 405 nm and collecting the emission in the range of 450-490 nm (represented in green), and the FM 4-64 fluorescent channel was observed by excitation at 561 nm and collecting the emission in the range of 640-700 nm (represented in red). The scale bars are 20 μm. (b) Gray value curves represent fluorescence intensity profile of the white line in its left-side fluorescent micrograph for both channels.

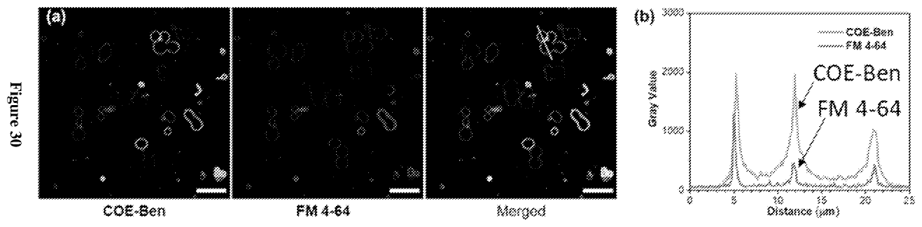

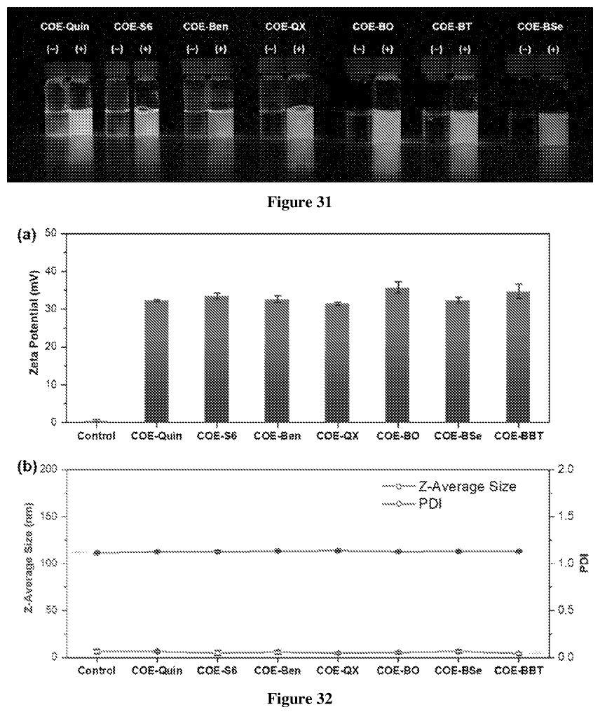

FIG. 31 shows photograph of COEs without (−) or with (+) 1 mM SUV treatment in PBS under UV-light (365 nm) exposure using a handheld UV lamp (UVP® UVLS-24 EL, 4 Watt). The concentration for COE-Quin is 0.25 μM; for COE-S6 and COE-Ben is 0.5 μM; for COE-QX, COE-BO and COE-BT is 1 μM; for COE-BSe is 5 μM. The camera (SONY, Alpha 7R) parameters are set to 8000 for ISO value, F2.8 for aperture, and 1/20 for exposure time.

FIG. 32 shows (a) zeta potential measurements for 1 mM POPC only SUVs stained by 5 μM of different COEs in DI water. (b) DLS measured Z-Average size and PDI for 1 mM POPC only SUVs stained by 5 μM of different COEs in DI water.

### Labelling Red Blood Cells Using COE for Microscopy and Flow Cytometry Analysis

Red blood cells were obtained from donor's blood after removing the plasma via centrifugation. The RBC were either used fresh or stored for 22 days. The packed RBC was diluted 500× into PBS before labelling with 2 μM of COE-S6 or 1 μg/mL of Cell Mask Deep Red. The cells were incubated with the dyes for 10 min before centrifuging down at 700 g for 3 min to remove free dyes. The labelled cell pellet was then resuspended in 1 mL of PBS. The cells were then diluted 10× further in PBS before imaging on the 8-well ibidi chamber slides, and analysing for size and fluorescence on the flow cytometry (Cytoflex).

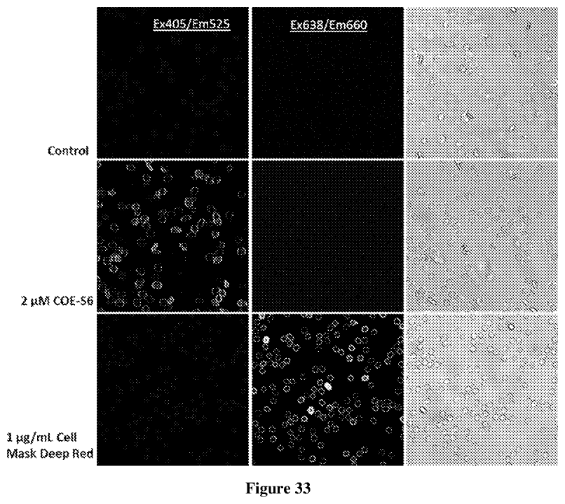

FIG. 33 shows confocal microscopy of fresh red blood cells (RBC) that were (a) unstained, (b) labeled with 2 μM COE-S6 and (c) 1 μg/mL Cell Mask Deep Red. Images were acquired on the Ex405/Em525 channel, Ex638/Em660 channel and brightfield channel.

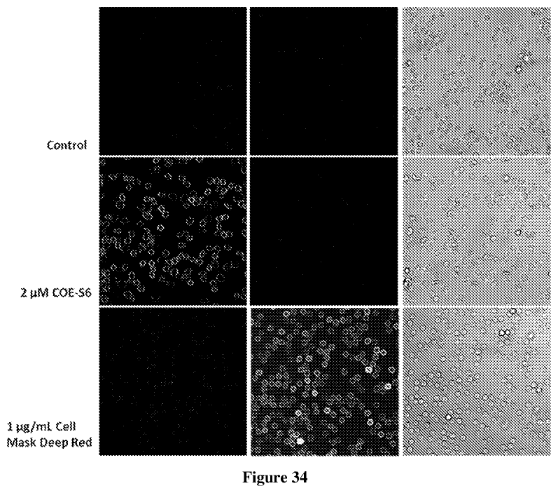

FIG. 34 shows confocal microscopy of red blood cells (RBC) that were stored for 22 days and (a) unstained, (b) labeled with 2 μM COE-S6 and (c) 1 μg/mL Cell Mask Deep Red. Images were acquired on the Ex405/Em525 channel, Ex638/Em660 channel and brightfield channel.

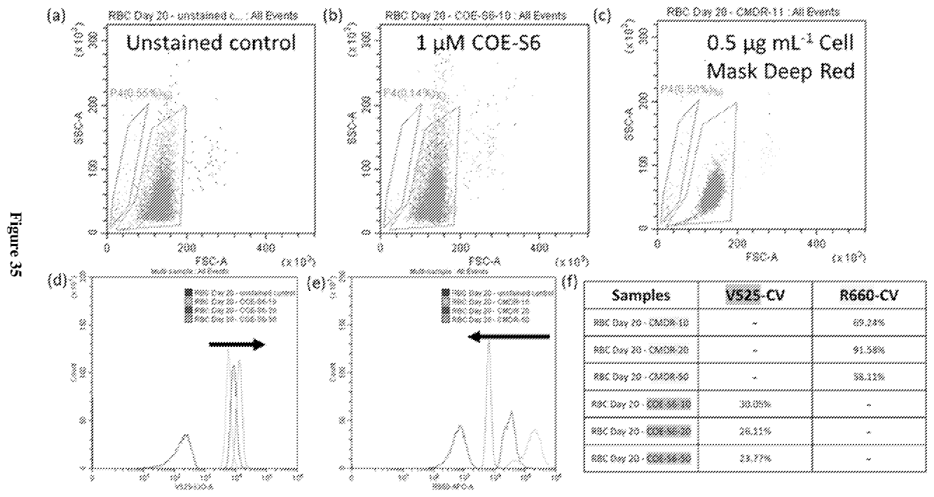

FIG. 35 shows FSC/SSC dot plots of red blood cells (RBC) measured on flow cytometry that are (a) unstained, (b) labelled with 1 μM COE-S6 and (c) 0.5 μg mL-1 Cell Mask Deep Red. COE-S6 labelling does not introduce an artifact to the morphology of the RBC unlike Cell Mask Deep Red. (d) Histograms showing the increase in fluorescence when RBCs were labelled with increasing COE-S6 concentrations and (e) the decrease in fluorescence when RBCs were labelled with increasing Cell Mask Deep Red due to quenching effect. COE-S6 labelling is also more homogeneous and uniform as seen from the coefficient of variance (CV) i.e., spread of the histogram data (f).

### NIR-II COE as Fluorescent Probe for Mammalian Cells

For the cell detection using near infrared fluorescence, cells were stained by 20 μM COE-BBT directly in DMEM media and incubated overnight. The stained cells were rinsed using warmed PBS (37° C.), followed by the addition of 1× Trypsin-EDTA. The cells were left to incubate in the 5% CO2 chamber at 37° C. for up to 5 minutes. DMEM media containing 10% FBS was added and cells were centrifuged at 200×g for 4 minutes. After removal of supernatant, cells were resuspended in media and taken for flow cytometry. COE-BBT-stained cells was excited using 808 nm laser. To collect the emission signal from COE-BBT, a 900 nm long-pass filter based on UV fused silica glass was customized (Chroma, INC. China), according to the CytoFLEX Platform Optical Filter Specifications (14.5±0.1 mm in length, 6.1±0.1 mm in width, 2.0±0.1 mm in thickness). This filter glass was mounted in a holder and was used in place of the IR885 channel (Ex808/Em885). The new channel to detect COE-BBT fluorescence was named as “IR1000”.

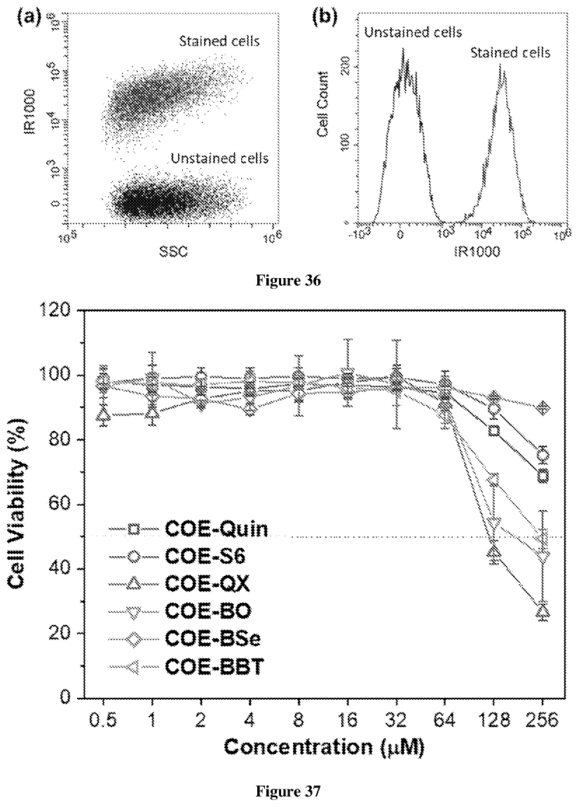

FIG. 36 shows flow cytometry measurement of COE-BBT-stained A549 cells by excitation using 808 nm laser and collecting the emission using 950 nm long-pass filter, which was named as IR1000 channel. Unstained A549 cells were employed as negative control.

### Cytotoxicity and Haemolytic Activity of COEs

A549 cells were routinely maintained in DMEM supplemented with 10% FBS, i.e. the cell culture medium (CCM). Stock solutions of COE were prepared by dissolving the solids in PBS to achieve 2 mM stock concentrations. The working COE solution at 256 μM was further prepared by diluting the stock solution in CCM. The stock solution of DiR was prepared in ethanol due to its poor water solubility. The working solution at 256 μM was prepared by dilution into CCM, which will give a final ethanol content of 12.8% (v/v). To test for cytotoxicity, 1000 cells were seeded into 96-well black plates with clear optical bottom, and left overnight in the 5% CO₂ chamber at 37° C. to adhere. The next day, different concentrations of COE/DiR solutions were achieved by two-fold serial dilution in CCM (concentrations ranged from 0.5-256 μM), and then used to replace the spent CCM in the wells. To delineate the toxic effects that may come from ethanol, the same dilution steps were also performed with neat ethanol instead of the ethanolic DiR dye solution, to achieve similar ethanol content in the respective wells. The cells were then left to incubate with the additives for 24 hours before cell viability was measured either using the CellTiter-Glo® (COE-Quin, COE-S6, COE-Ben, COE-BBT, DiR) or CCK-8 (COE-BT, COE-BO, COE-QX, COE-BSe) according to the manufacturer's protocol. Luminscence (CellTiter-Glo® assay) and absorbance (CCK-8 assay) measurements were recorded using the TECAN plate reader (Spark®). The cytotoxicity assay was done in triplicates.

FIG. 37 shows cytotoxicity measurements against A549 cells for COEs. The IC50 values for all six COEs are higher than 100 μM.

Bovine whole blood was purchased from Innovative Research, USA. 2 mL blood was mixed with 10 mL PBS and centrifuged at 1,000 rpm for 5 min. Red blood cell pellets were collected and subsequently washed with PBS three times, and then diluted using PBS to a concentration of 2% (v/v). Only COE-Quin, COE-S6 and COE-Ben were selected in this study, due to they have insignificant absorption interference at 540 nm. Each COE was dissolved in PBS and two folds serial diluted in a 96-well microplate. 100 μL red blood cell suspension was mixed with 100 μL COE solution in each well and incubated for 1 hour at 37° C. under shaking (200 rpm). The microplate was centrifuged at 1,000 rpm for 10 min. 150 μL aliquots of the supernatant were transferred to a new 96-well microplate. Hemolytic activity was calculated by measuring absorbance at 540 nm using the Multimode Microplate Reader (Spark®, Tecan). Triton X-100 (0.1% in PBS) which is able to lyse red blood cells completely was used as a positive control, while bovine erythrocytes in PBS was used as a negative control. The hemolysis percentage was calculated using the following formula:

$$Hemolysis ( \% ) = \frac{{O}_{c} - {O}_{b}}{{O}_{t} - {O}_{b}} \times 100 \%$$

where O_{c} is the absorbance of COE-treated sample, O_{b} is the absorbance of negative control and Oₜ is the absorbance of positive control. The hemolysis assay was done in four replicates.

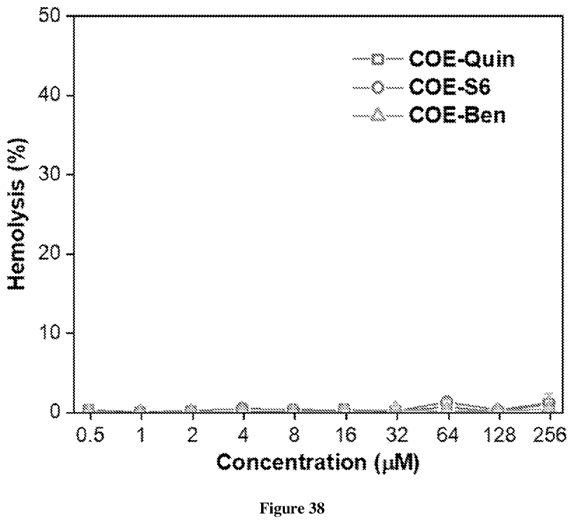

FIG. 38 shows hemolysis measurements for COEs against bovine erythrocytes in PBS.

1. A compound of Formula (I) or a salt or solvate thereof: 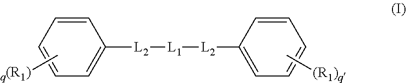
wherein
each R₁ is independently selected from optionally substituted alkoxy, optionally substituted alkenyloxy, optionally substituted alkynyloxy;
q is an integer selected from 1 to 5;
q′ is an integer selected from 1 to 5;
wherein
each L₂ is independently selected from optionally substituted ethylene, or optionally substituted phenylethylene;
L₁ is a π-conjugated core comprising monomeric unit A and monomeric unit D: 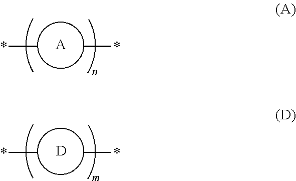
wherein
A is selected from 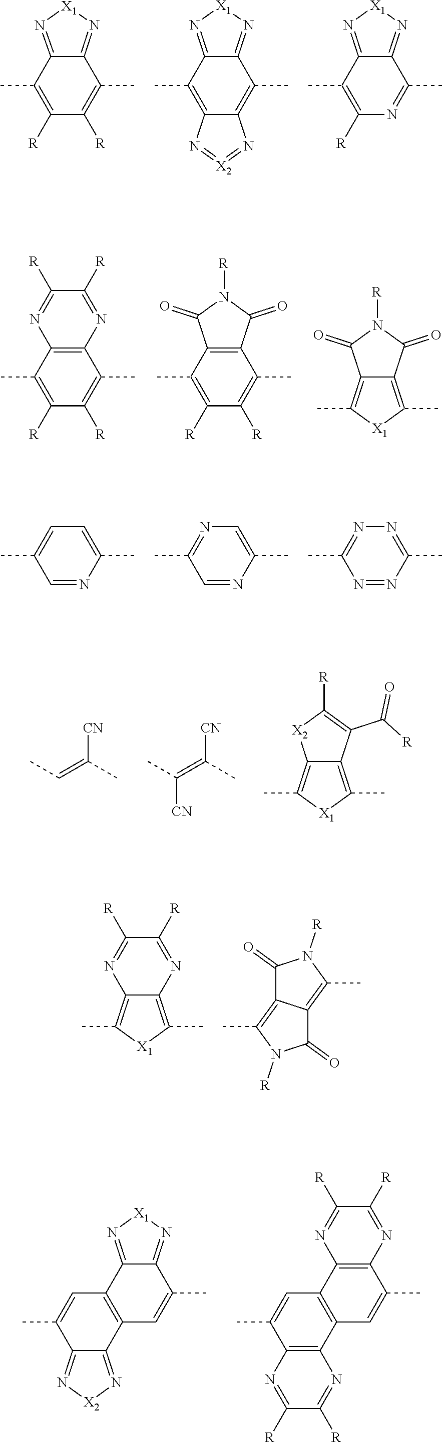 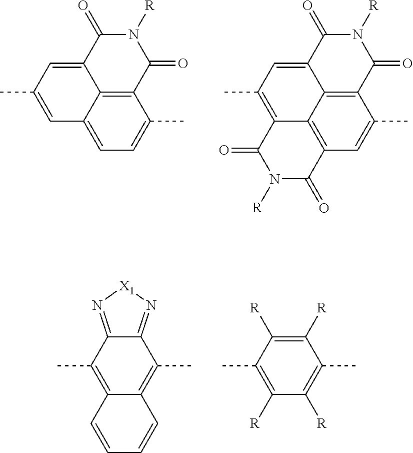
wherein [symbol] represents a bond to D or to L₂;
D is selected from 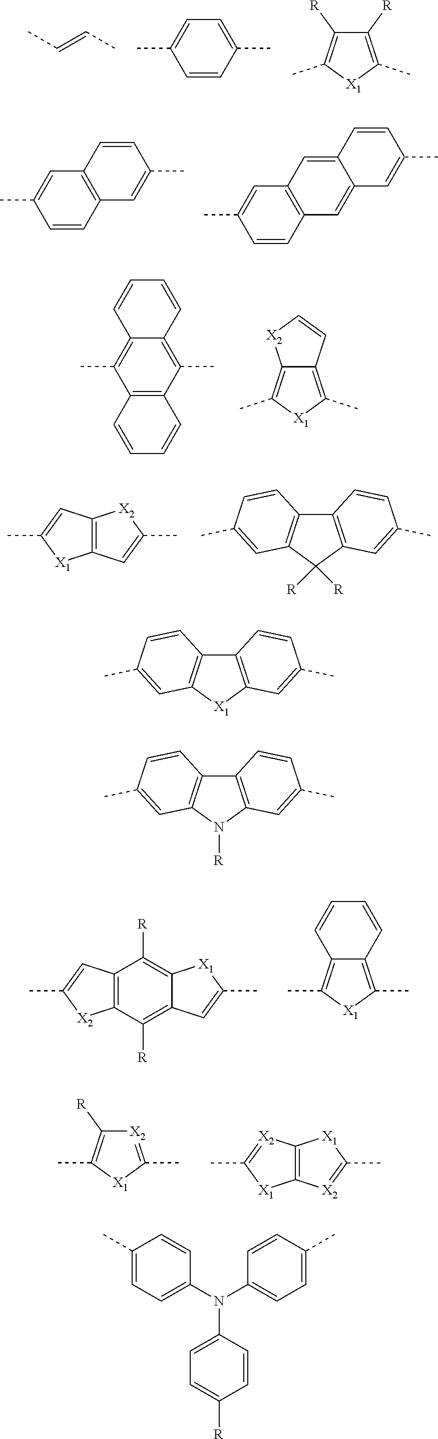 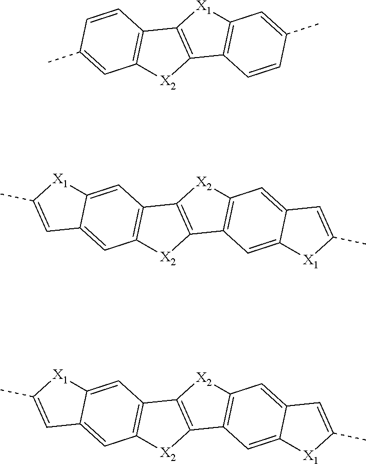
wherein [symbol] represents a bond to A or to L₂;
each X₁ is independently selected from C, O, N, S and Se;
each X₂ if present is independently selected from C, O, N, S and Se;
when X₂ is present, at least one of X₁ and X₂ is O, N or S;
each R is independently selected from H, halo, cyano, and optionally substituted alkyl;
n is an integer selected from 1 to 5;
m is an integer selected from 1 to 5;
wherein * represents a bond to another monomeric unit or to L₂;
wherein monomeric units A and monomeric units D are alternatively bonded to each other;
wherein L₁ is not butadienylene, polyalkenylene, phenylalkenylene and polyphenylalkenylene.

2. The compound according to claim 1, wherein A is an electron accepting moiety.

3. (canceled)

4. The compound according to claim 1, wherein A is a moiety of Formula (II): 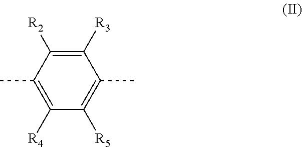
wherein [symbol] represents a bond to D or to L₂;
R₂, R₃, R₄ and R₈ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₂ and R₃ are linked to form optionally substituted heterocyclyl, optionally substituted heteroaryl; or
R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl.

5. The compound according to claim 1, wherein A is 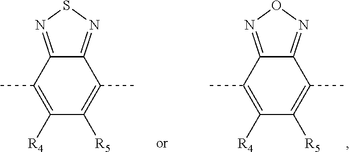
wherein R₄ and R₈ are independently selected from H, halogen, optionally substituted alkyl, optionally substituted alkenyl, optionally substituted alkynyl, optionally substituted alkoxy, optionally substituted alkenyloxy, optionally substituted alkynyloxy; or R₄ and R₅ are linked to form optionally substituted cycloalkyl, optionally substituted cycloalkenyl, optionally substituted cycloalkynyl, optionally substituted heterocyclyl, optionally substituted aryl, optionally substituted heteroaryl.

6. The compound according to claim 1, wherein A is

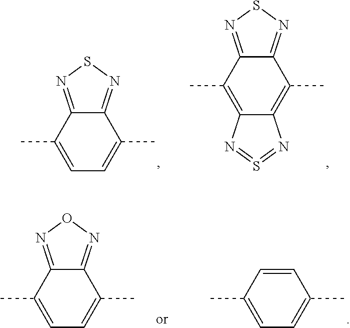

7. The compound according to claim 1, wherein D is an electron donating moiety.

8. (canceled)

9. The compound according to claim 1, wherein D is an optionally substituted 5 membered heteroarylene or phenylene.

10. The compound according to claim 1, wherein D is a moiety of Formula (III): 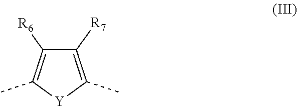
wherein Y is NR, O, or S;
R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl; and
R is selected from H, halo, cyano, and optionally substituted alkyl.

11. The compound according to claim 1, wherein D is 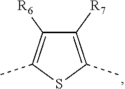
wherein R₆ and R₇ are independently selected from H, halogen, optionally substituted alkyl, optionally substituted alkenyl, optionally substituted alkynyl, optionally substituted alkoxy, optionally substituted alkenyloxy, optionally substituted alkynyloxy; or
R₆ and R₇ are linked to form optionally substituted cycloalkyl, optionally substituted cycloalkenyl, optionally substituted cycloalkynyl, optionally substituted heterocyclyl, optionally substituted aryl, optionally substituted heteroaryl.

12. The compound according to claim 1, wherein D is

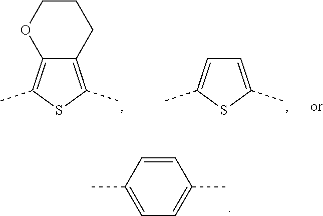

13. The compound according to claim 1, wherein L₁ is selected from:

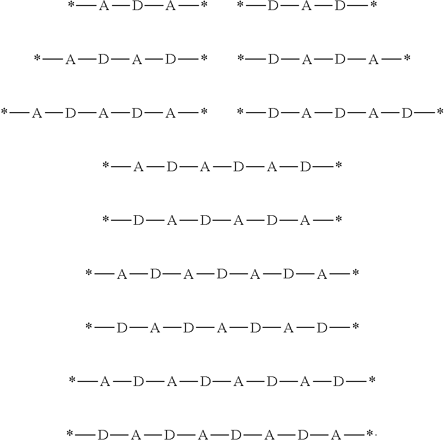

14. The compound according to claim 1, wherein R₁ is independently optionally substituted alkoxy.

15. (canceled)

16. The compound according to claim 1, wherein R₁ is independently C₃-C₈ alkoxy substituted with amino, or alkylamino.

17. The compound according to claim 1, wherein the optional substituent on L₁ is independently selected from halogen, cyano, optionally substituted alkyl, optionally substituted alkenyl or optionally substituted alkynyl.

18. The compound according to claim 1, wherein the compound of Formula (I) is a compound of Formula (Ia):

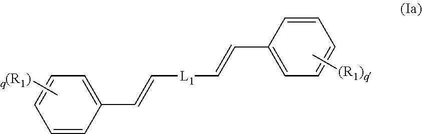

19. The compound according to claim 1, wherein the compound of formula (I) is a compound of Formula (Ib):

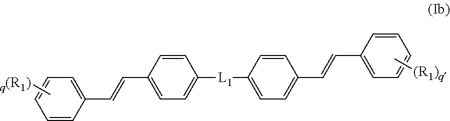

20-25. (canceled)

26. The compound according to claim 1, wherein the compound of Formula (I) is selected from

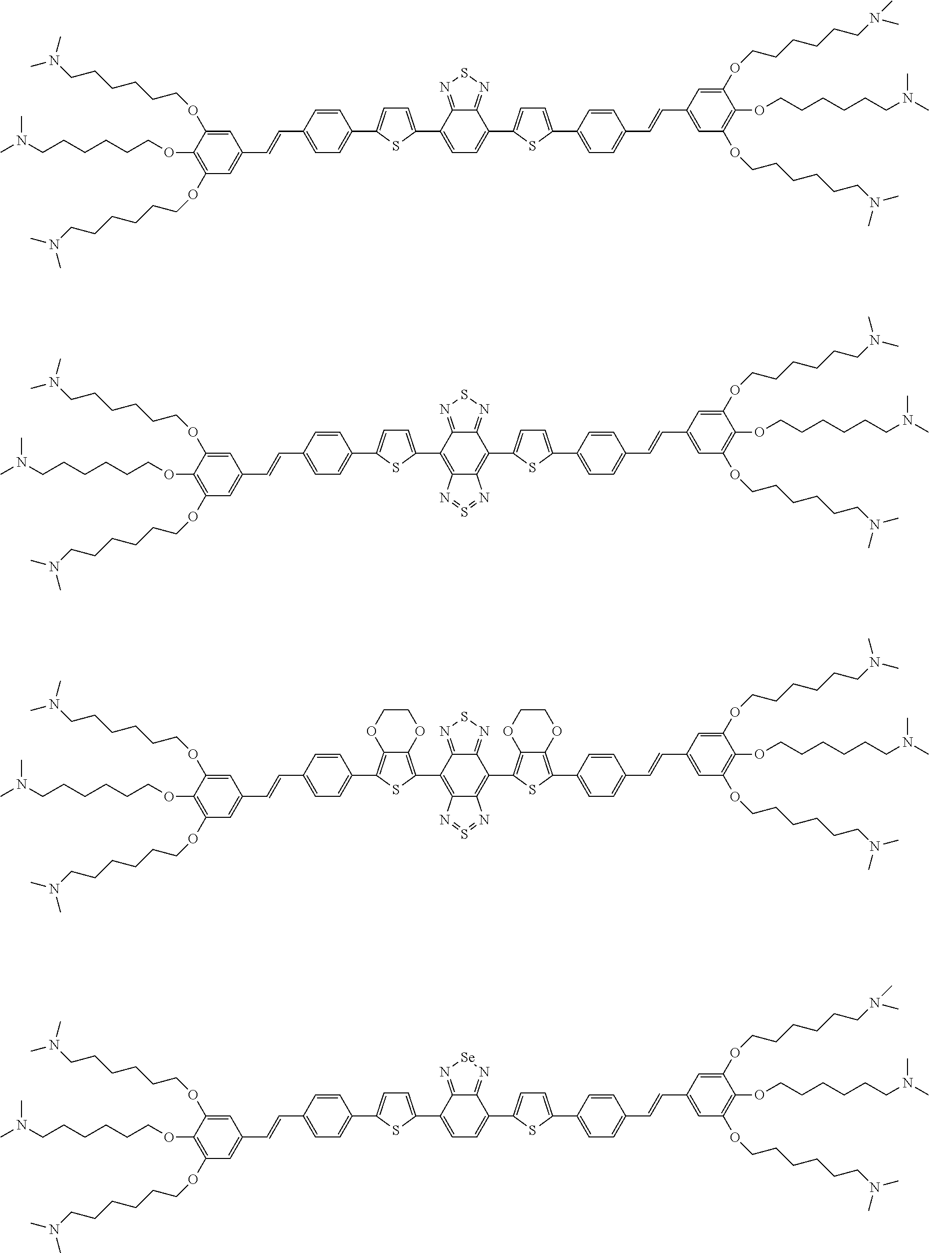

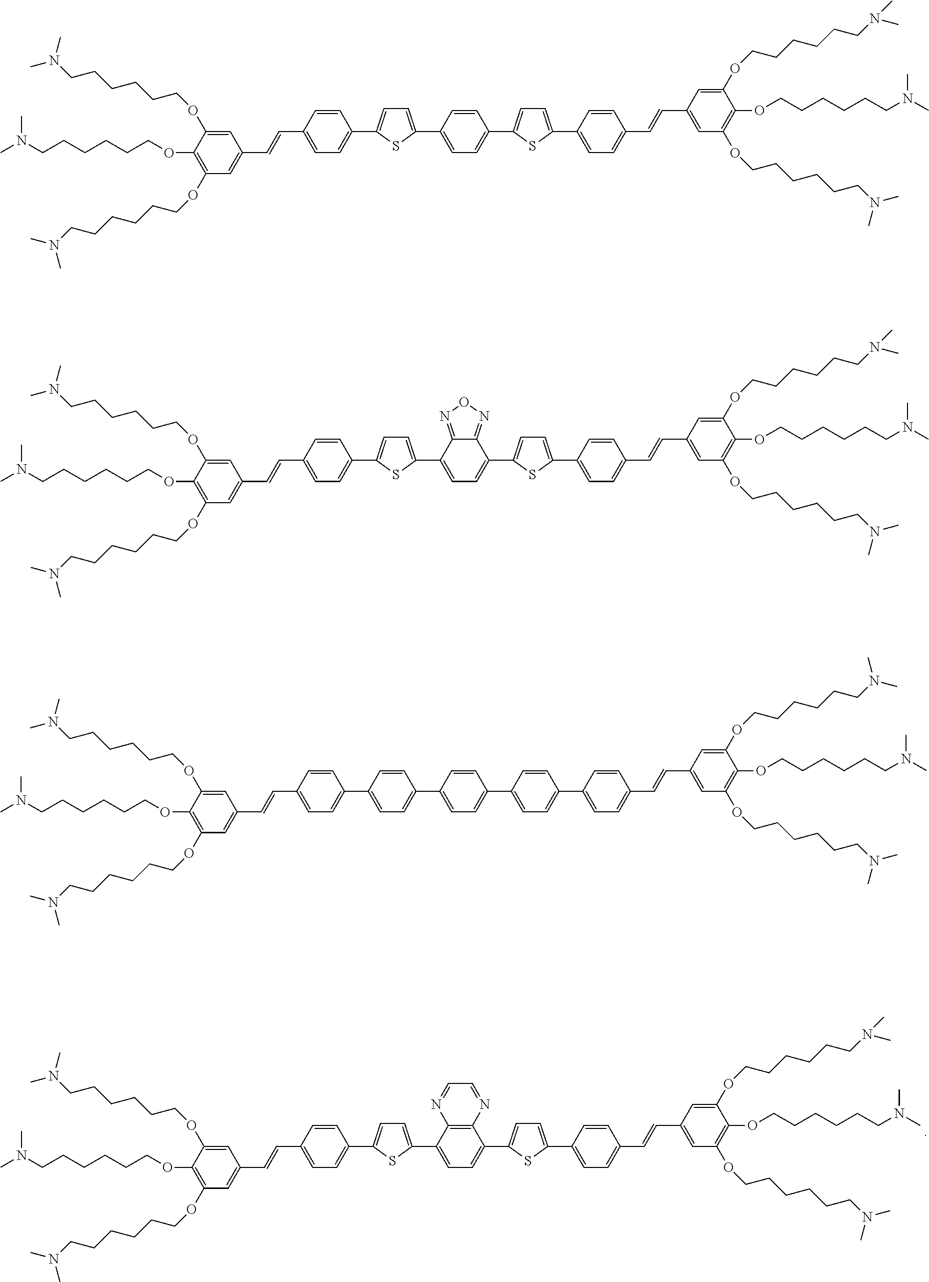

27. The compound according to claim 1, wherein the compound of Formula (I) is a compound of Formula (Ig) or a salt or solvate thereof: 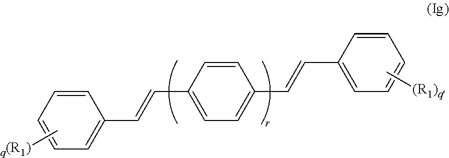
wherein
r is an integer selected from 1 to 5.

28. A method of labelling a cell and/or a lipid vesicle, comprising:
a) incubating a compound of Formula (I), or a salt or solvate thereof with the cell and/or a lipid vesicle.

29-39. (canceled)

40. A lipid vesicle and/or liposome, comprising a compound of Formula (I) or a salt or solvate thereof according to claim 1.
